SEQUENCE LISTING (41 sequences)

SEQ ID NO: 1 - protein, 450 aa, synthetic construct
EVQLVESGGGLVKPGGSLRLSCAASGFTFSTYGVSWVRQAPGKGLEWVSTISSGGGYTHYAGSVKGRFTISRDNAKNSLYLQMNSLRAEDTAVYYCARHPRDFSYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
EIVMTQSPATLSVSPGERATLSCRASQDVGHYLAWYQQKPGQAPRLLIYWASTRATGIPARFSGSGSGTEFTLTISSLQSEDFAVYYCQQYNIYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, synthetic construct
EVQLVESGGGLVKPGGSLRLSCAASGFTFSTYGVSWVRQAPGKGLEWVSTISSGGGYTHYAGSVKGRFTISRDNAKNSLYLQMNSLRAEDTAVYYCARHPRDFSYALDYWGQGTTVTVSS

SEQ ID NO: 4 - protein, 107 aa, synthetic construct
EIVMTQSPATLSVSPGERATLSCRASQDVGHYLAWYQQKPGQAPRLLIYWASTRATGIPARFSGSGSGTEFTLTISSLQSEDFAVYYCQQYNIYPWTFGQGTKVEIK

SEQ ID NO: 5 - protein, 8 aa, synthetic construct
GFTFSTYG

SEQ ID NO: 6 - protein, 8 aa, synthetic construct
ISSGGGYT

SEQ ID NO: 7 - protein, 13 aa, synthetic construct
ARHPRDFSYALDY

SEQ ID NO: 8 - protein, 6 aa, synthetic construct
QDVGHY

SEQ ID NO: 9 - skipped

SEQ ID NO: 10 - protein, 9 aa, synthetic construct
QQYNIYPWT

SEQ ID NO: 11 - protein, 450 aa, synthetic construct
EVQLVESGGGLVKPGGSLRLSCAASGFTFSQYGHSWVRQAPGKGLEWVSTISSGGGYTHYAHSVKGRFTISRDNAKNSLYLQMNSLRAEDTAVYYCARHPRDFSYANDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 120 aa, synthetic construct
EVQLVESGGGLVKPGGSLRLSCAASGFTFSQYGHSWVRQAPGKGLEWVSTISSGGGYTHYAHSVKGRFTISRDNAKNSLYLQMNSLRAEDTAVYYCARHPRDFSYANDYWGQGTTVTVSS

SEQ ID NO: 13 - protein, 8 aa, synthetic construct
GFTFSQYG

SEQ ID NO: 14 - protein, 13 aa, synthetic construct
ARHPRDFSYANDY

SEQ ID NO: 15 - protein, 450 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, synthetic construct
DIVMTQSHKFMSTSIGDRVSITCKASQDVGHYVAWYQQKPGQSPKLLIYWASTRHTGVPDRFTGSGSGTDFTLTISNVQSEDLADYFCQQYNIYPWTFGGGSKLAIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 120 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSS

SEQ ID NO: 18 - protein, 107 aa, synthetic construct
DIVMTQSHKFMSTSIGDRVSITCKASQDVGHYVAWYQQKPGQSPKLLIYWASTRHTGVPDRFTGSGSGTDFTLTISNVQSEDLADYFCQQYNIYPWTFGGGSKLAIK

SEQ ID NO: 19 - protein, 8 aa, synthetic construct
GFTFSNYG

SEQ ID NO: 20 - protein, 13 aa, synthetic construct
ARHPRDFSYAMDY

SEQ ID NO: 21 - protein, 450 aa, synthetic construct
EVQLVESGGDLVKPGGSLKLSCAASGFTFSNYGMSWVRQTPDKRLEWVATISSGGGYTHYVDSVKGRFTISRDNANHILYLQMSSLNSEDTAMYYCARHPRDFSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, synthetic construct
TYGVS

SEQ ID NO: 23 - protein, 17 aa, synthetic construct
TISSGGGYTHYAGSVKG

SEQ ID NO: 24 - protein, 11 aa, synthetic construct
HPRDFSYALDY

SEQ ID NO: 25 - protein, 11 aa, synthetic construct
RASQDVGHYLA

SEQ ID NO: 26 - protein, 7 aa, synthetic construct
WASTRAT

SEQ ID NO: 27 - protein, 7 aa, synthetic construct
GFTFSTY

SEQ ID NO: 28 - protein, 6 aa, synthetic construct
SSGGGY

SEQ ID NO: 29 - protein, 13 aa, synthetic construct
AASGFTFSTYGVS

SEQ ID NO: 30 - protein, 10 aa, synthetic construct
TISSGGGYTH

SEQ ID NO: 31 - protein, 8 aa, synthetic construct
YWASTRAT

SEQ ID NO: 32 - protein, 6 aa, synthetic construct
STYGVS

SEQ ID NO: 33 - protein, 13 aa, synthetic construct
WVSTISSGGGYTH

SEQ ID NO: 34 - protein, 12 aa, synthetic construct
ARHPRDFSYALD

SEQ ID NO: 35 - protein, 7 aa, synthetic construct
GHYLAWY

SEQ ID NO: 36 - protein, 10 aa, synthetic construct
LLIYWASTRA

SEQ ID NO: 37 - protein, 8 aa, synthetic construct
QQYNIYPW

SEQ ID NO: 38 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 39 - protein, 4 aa, synthetic construct
GGFG

SEQ ID NO: 40-41 - skipped (2 entries)

(19) United States
(12) Patent Application Publication
(10) Pub. No.: US 2026/0007762 A1
(43) Pub. Date: Jan. 8, 2026

(54) ANTI-ROR2 ANTIBODY-DRUG CONJUGATES

(71) Applicant: Solve Therapeutics, Inc., San Diego, CA (US)

(72) Inventors: Brian LANNUTTI, Solana Beach, CA (US); Jeffry D. WATKINS, Encinitas, CA (US); Katti JESSEN, San Diego, CA (US)

(73) Assignee: Solve Therapeutics, Inc., San Diego, CA (US)

(21) Appl. No.: 19/260,021

(22) Filed: Jul. 3, 2025

Related U.S. Application Data
(60) Provisional application No. 63/667,545, filed on Jul. 3, 2024.

(51) Int. Cl. A61K 47/68; A61K 45/06; A61P 35/00; C07K 16/28

(52) U.S. Cl. CPC A61K 47/68031; A61K 45/06; A61K 47/68037; A61K 47/6849; A61K 47/6889; A61P 35/00; C07K 16/2803

(57) ABSTRACT

The present disclosure provides antibody-drug conjugates comprising an anti-ROR2 antibody or an antigen-binding portion thereof and a cytotoxic drug moiety, and methods of using them to treat ROR2-expressing cancers.

### CROSS-REFERENCE TO RELATED APPLICATION

This application claims priority to U.S. Provisional Patent Application No. 63/667,545, filed Jul. 3, 2024. The disclosure of the priority application is incorporated by reference herein in its entirety.

### SEQUENCE LISTING

The instant application contains a Sequence Listing which has been submitted electronically in XML format and is hereby incorporated by reference herein in its entirety. The electronic copy of the Sequence Listing, created on Jun. 26, 2025, is named 122878.US017.xml and is 38,158 bytes in size.

### BACKGROUND OF THE INVENTION

Cancer is the second leading cause of human death next to heart disease. Receptor tyrosine kinases (RTKs) play a key role in oncogenic transformation, as well as cancer growth and metastasis, by regulating cell differentiation, proliferation, migration, angiogenesis, and survival. Receptor tyrosine kinase-like orphan receptor 2 (“ROR2”) is a cell membrane protein and is a receptor for Wnt5a, a proinflammatory factor in human ovarian granulosa cells. ROR2 modulates Wnt signaling through sequestration of Wnt ligands and can also repress transcription of Wnt target genes involved in tumor suppression. ROR2 has been implicated in the progression of numerous cancers, including breast, ovarian, pancreatic, cervical, gastric, renal, head and neck, bone, skin, and prostate cancers. Accordingly, ROR2 is of interest as a target for anti-cancer immunotherapies.

In view of the role of ROR2 in cancer, there is a need for new and improved therapies that target ROR2-positive cancer cells.

### SUMMARY OF THE INVENTION

The present disclosure provides antibody-drug conjugates (i.e., immunoconjugates) that comprise an anti-ROR2 antibody or an antigen-binding portion thereof conjugated (e.g., by way of a linker) to a cytotoxic drug moiety. The immunoconjugate may have the formula of Ab−((L)m−(D))n, wherein:
Ab is an antibody or an antigen-binding portion thereof that specifically binds to human receptor tyrosine kinase like orphan receptor 2 (ROR2);
L is a linker;
m is 0 or 1;
D is a cytotoxic drug moiety; and
n is an integer from 1 to 10.

In some embodiments, the cytotoxic drug moiety of an immunoconjugate herein may be an anti-tubulin agent or a topoisomerase I inhibitor. For example, the cytotoxic drug moiety may comprise a chemical structure selected from the group consisting of monomethyl auristatin E (MMAE), monomethyl auristatin F (MMAF), exatecan, an exatecan derivative (DXd), and SN-38, or a pharmaceutically acceptable salt, analog, prodrug, or, if appropriate, ester thereof.

In some embodiments, the linker of an immunoconjugate herein comprises a cleavable moiety. In certain embodiments, the linker comprises one or more of valine-alanine (VA), valine-citrulline (VC), C5-C(═O)—VC, para-substituted phenylene-C3 (Ph(p)-C3), meta-substituted phenylene-C2 (Ph(m)-C2), C5 alkyl, para-aminobenzyloxycarbonyl (PAB), amino methylene (AM), and GGFG (SEQ ID NO: 39). In certain embodiments, the linker comprises a formula selected from Formula (I), (Ia), (Ib), (Ic), (Id), (Ie), (If), (II), (IIa), (IIb), (IIc), (IId), (IIe), and (IIf), or a pharmaceutically acceptable salt, or if appropriate, ester, thereof.

In some embodiments, an immunoconjugate herein comprises a linker comprising C₅—C(═O)—VC—PAB; Ph(p)-C₃—C(═O)-GGFG; Ph(m)-C₂—C(═O)-GGFG; or C₅—C(═O)-GGFG. In some embodiments, an immunoconjugate herein comprises a formula selected from Formula (I), (Ia), (Ib), (Ic), (Id), (Ie), and (If), where m is 2 and Y is a valine-alanine dipeptide, or a pharmaceutically acceptable salt, or if appropriate, ester, thereof. In some embodiments, an immunoconjugate herein comprises a formula selected from Formula (II), (IIa), (IIb), (IIc), (IId), (IIe), and (IIf), or a pharmaceutically acceptable salt, or if appropriate, ester, thereof, wherein X comprises Formula (A), (B), or (C).

In some embodiments, an immunoconjugate herein has a structure shown in Table 3B as Formula (IIIa) (SLV-A), Formula (IVa) (SLV-B, -D, and -L), Formula (Va) (SLV-E), Formula (IXa) (SLV-M), Formula (Xa) (SLV-N), or Formula (XIa) (SLV-O and —P), or a pharmaceutically acceptable salt, or if appropriate, ester, thereof. In particular embodiments, an immunoconjugate herein has a structure shown in Table 3B as
Formula (VIa), Formula (VIa.1), Formula (VIa.2) (SLV-I),
Formula (VIIa), Formula (VIIa.1), Formula (VIIa.2) (SLV-J),
Formula (VIII.1a), Formula (VIII.2a), Formula (VIII.3a), Formula (VIII.4a), Formula (VIII.5a),
Formula (VIII.6a), or Formula (VIII.7a) (SLV-K), or a pharmaceutically acceptable salt, or if appropriate, ester, thereof.

In some embodiments, the ratio of the cytotoxic drug moiety to the antibody (DAR) for an immunoconjugate herein is 3 to 8.

In some embodiments of an immunoconjugate herein, the antibody or antigen-binding portion of the immunoconjugate competes or cross-competes for binding to human ROR2, or binds to the same human ROR2 epitope, as an antibody that comprises a heavy chain (HC) and a light chain (LC) comprising SEQ ID NOs: 1 and 2, respectively; SEQ ID NOs: 11 and 2, respectively; SEQ ID NOs: 15 and 16, respectively; or SEQ ID NOs: 21 and 16, respectively.

In certain embodiments, the antibody or antigen-binding portion comprises heavy chain complementarity-determining region (CDR) 1-3 (HCDR1-3) and light chain CDR1-3 (LCDR1-3) amino acid sequences of SEQ ID NOs: 5, 6, 7, 8, 9, and 10, respectively; SEQ ID NOs: 13, 6, 14, 8, 9, and 10, respectively; or SEQ ID NOs: 19, 6, 20, 8, 9, and 10, respectively.

In certain embodiments, the antibody or antigen-binding portion comprises HCDR1 comprising SEQ ID NO: 40; HCDR2 comprising SEQ ID NO: 28; HCDR3 comprising SEQ ID NO: 24; LCDR1 comprising SEQ ID NO: 41; LCDR2 comprising SEQ ID NO: 9; and LCDR3 comprising SEQ ID NO: 37. The antibody or antigen-binding portion may comprise HCDR1-3 and LCDR1-3 of 5, 6, 7, 8, 9, and 10, respectively, or HCDR1-3 and LCDR1-3 of 22, 23, 24, 25, 26, and 10, respectively.

In certain embodiments, the antibody or antigen-binding portion comprises heavy chain variable domain (V_{H}) and light chain variable domain (V_{L}) amino acid sequences of SEQ ID NOs: 3 and 4, respectively; SEQ ID NOs: 12 and 4, respectively; or SEQ ID NOs: 17 and 18, respectively.

In some embodiments, the antibody is of isotype IgG. For example, the antibody may be of isotype subclass IgG₁, IgG₂, IgG₃, or IgG₄. In certain embodiments, the Fc region of the antibody comprises one or more mutations that reduce effector function.

In particular embodiments, the antibody comprised by an immunoconjugate herein comprises HC and LC amino acid sequences of SEQ ID NOs: 1 and 2, respectively; SEQ ID NOs: 11 and 2, respectively; SEQ ID NOs: 15 and 16, respectively; or SEQ ID NOs: 21 and 16, respectively; optionally wherein the HC amino acid sequence lacks the C-terminal lysine.

In some embodiments, the antigen-binding portion comprised by an immunoconjugate herein is a Fab, F(ab)₂, or scFv.

The present disclosure also provides a pharmaceutical composition comprising an immunoconjugate herein and a pharmaceutically acceptable excipient. In some embodiments, the pharmaceutical composition may further comprise an additional therapeutic agent, such as an immunomodulatory agent, a chemotherapeutic agent, an anti-neoplastic agent, or an anti-angiogenic agent.

The present disclosure also provides a method of treating cancer in a human patient in need thereof, comprising administering to the patient a therapeutically effective amount of an immunoconjugate herein. In some embodiments, the cancer expresses ROR2. In some embodiments, the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma. The method may further comprise administering to the patient an additional therapeutic agent, for example, an immunomodulatory agent, a chemotherapeutic agent, an anti-neoplastic agent, an anti-angiogenic agent, or a tumor vaccine.

The present disclosure also provides an immunoconjugate herein, or a pharmaceutical composition herein, for use in treating cancer in a method described herein.

The present disclosure also provides use of an immunoconjugate herein, or a pharmaceutical composition herein, for treating cancer in a method described herein.

The present disclosure also provides use of an immunoconjugate herein, or a pharmaceutical composition herein, in the manufacture of a medicament for treating cancer in a method described herein.

The present disclosure also provides a method of making an immunoconjugate herein, comprising: providing an antibody or an antigen-binding portion thereof that specifically binds to human receptor tyrosine kinase like orphan receptor 2 (ROR2); conjugating to the antibody or antigen-binding portion a cytotoxic drug moiety selected from the group consisting of monomethyl auristatin E (MMAE), monomethyl auristatin F (MMAF), exatecan, an exatecan derivative (DXd), and SN-38; or a pharmaceutically acceptable salt, analog, prodrug, or, if appropriate, ester thereof; wherein the antibody or antigen-binding portion is as described herein.

It is understood that the present disclosure also provides an ADC or pharmaceutical composition described herein for use in treating a patient (e.g., a human patient) in need thereof in a therapeutic method described herein. Also provided are uses of an ADC or pharmaceutical composition described herein for the manufacture of a medicament for treating a patient (e.g., a human patient) in need thereof in a therapeutic method described herein.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a line graph showing the binding of the ADCs shown to non-small cell lung carcinoma H1155 tumor cells, and a table showing the EC₅₀ value for each ADC. ADC-I: SLV-I, ADC-J: SLV-J, etc. in the drawings. MFI: median fluorescence intensity.

FIG. 2 is a line graph showing the binding of the ADCs shown to non-small cell lung carcinoma H1155 tumor cells. MFI: median fluorescence intensity.

FIG. 3 is a set of line graphs showing the internalization rate of the ADCs shown in non-small cell lung carcinoma H1155 tumor cells. MFI: median fluorescence intensity.

FIG. 4 is a line graph showing the cell killing activity of the ADCs shown in non-small cell lung carcinoma H1155 tumor cells.

FIG. 5 is a set of line graphs showing the cell killing activity of the ADCs shown in osteosarcoma-derived HOS cells.

FIG. 6 is a line graph showing tumor volume over time in a triple negative breast cancer xenograft model (CTG-2215 cells) following treatment with the ADCs shown.

FIG. 7 is a line graph showing tumor volume over time in a non-small cell lung cancer xenograft model (LCLC-103H tumor cell line) following treatment with the ADCs shown.

FIG. 8 is a line graph showing tumor volume over time in a non-small cell lung cancer xenograft model (LCLC-103H tumor cell line) following treatment with the ADCs shown.

FIG. 9 is a line graph showing tumor volume over time in a non-small cell lung cancer xenograft model (H520 tumor cell line) following treatment with the ADCs shown.

FIG. 10 is a line graph showing tumor volume over time in a non-small cell lung cancer xenograft model (H520 tumor cell line) following treatment with the ADCs shown.

FIG. 11 is a line graph showing tumor volume over time in a chronic myeloid leukemia xenograft model (K562 tumor cell line) following treatment with the ADCs shown.

### DETAILED DESCRIPTION OF THE INVENTION

The present disclosure provides antibody-drug conjugates (ADCs) comprising an antibody or an antigen-binding portion thereof that specifically binds to ROR2. These ADCs can be used to treat cancer (e.g., a ROR2-positive cancer) in a patient. Compared to currently available treatments for cancer, it is contemplated that the ADCs and compositions described herein may provide a superior clinical response. Also provided are pharmaceutical compositions comprising one or more of the ADCs and use of the ADCs for treatment of cancer (e.g., ROR2-expressing cancer). The ADCs and compositions described herein may be used for treating cancer in a patient; may be used for the manufacture of a medicament for treating cancer in a patient; or may be for use in treating cancer in a patient. In particular embodiments, the patient is a human patient in need thereof.

### I. Immunoconiuwates

The terms “antibody-drug conjugate,” “ADC,” and “immunoconjugate” are used interchangeably herein and refer to an antibody or an antigen-binding portion thereof that is covalently or non-covalently bonded, with or without a linker, to one or more biologically active molecules (i.e., “drugs” or “drug moieties”). The conjugated biologically active molecules are also called “payloads.” The present ADCs comprise an antibody or a portion thereof that is specific for human ROR2 (“anti-ROR2 ADCs”) and thus can serve as targeting moieties for delivering conjugated payloads to ROR2-positive cancer cells. It is understood that where the present disclosure refers to an anti-ROR2 antibody or an antigen-binding portion thereof, any moiety that serves as a means for binding to ROR2 may be used.

Embodiments of the antibody or portion thereof, the linker, and the drug moiety used in the ADCs are described in further detail below.

### A. Antibodies and Antigen-Binding Portions Thereof

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (V_{H}) and a heavy chain constant region (C_{H}). Each light chain is composed of a light chain variable domain (V_{L}) and a light chain constant region (C_{L}). The V_{H} and V_{L} domains can be subdivided further into regions of hypervariability, termed “complementarity-determining regions” (CDRs), interspersed with regions that are more conserved, termed “framework regions” (FRs). Each V_{H} and V_{L} is composed of three CDRs (HCDR herein designates a CDR from the heavy chain; and LCDR herein designates a CDR from the light chain) and four FRs, arranged from amino-terminus to carboxy-terminus in the following order: FR1, CDR1, FR2, CDR2, FR3, CDR3, FR4. The amino acid position numbers and FRs and CDRs in the heavy or light chain may be defined in accordance with the IMGT® system (Lefranc et al., Dev Comp Immunol. (2003) 27(1):55-77); or the Kabat system (Sequences of Proteins of Immunological Interest, National Institutes of Health, Bethesda, MD (1987 and 1991)); the Chothia system (Chothia & Lesk, J Mol Biol. (1987) 196:901-17; Chothia et al., Nature (1989) 342:878-83); Abhinandan et al., Molecular Immunology (2008) 45(14):3832-39; MacCallum et al., J Mol Biol. (1996) 262:732-45; or Honegger and Plickthun, J Mol Biol. (2001) 309(3):657-70.

The term “recombinant antibody” refers to a non-naturally occurring antibody that is expressed from a cell or cell line comprising one or more nucleotide sequences that encode the antibody, wherein the cell or cell line does not naturally comprise the nucleotide sequence(s).

The term “isolated protein,” “isolated polypeptide” or “isolated antibody” refers to a protein, polypeptide or antibody that by virtue of its origin or source of derivation (1) is not associated with components that naturally accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between two molecules, e.g., between an antigen and an antibody. The intrinsic attractiveness of an antibody for an antigen is typically expressed as the binding affinity equilibrium constant (K_{D}) of the antibody-antigen interaction. An antibody is said to specifically bind to an antigen when the K_{D} for the binding is ≤1 μM, e.g., ≤100 nM or ≤10 nM. A K_{D} binding affinity constant can be measured, e.g., by surface plasmon resonance (SPR) using, for example, the Biacore™ T200 system, the IBIS-MX96 SPR system from IBIS Technologies, or the Carterra LSA SPR platform, or by bio-layer interferometry (BLI) using, for example, the Octet™ system from ForteBio.

The term “epitope” as used herein refers to a portion (determinant) of an antigen that specifically binds to an antibody or a related molecule such as a bispecific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., ROR2) or a relevant portion thereof, then screening for binding to the epitope. An antibody to a particular epitope also may be generated using phage display methods.

One can determine whether an antibody binds to the same epitope as or competes for binding with an anti-ROR2 antibody of the present disclosure by using methods known in the art, including, without limitation, competition assays, epitope binning, and alanine scanning. In some embodiments, one allows the anti-ROR2 antibody of the present disclosure to bind to ROR2 under saturating conditions, and then measures the ability of the test antibody to bind to ROR2. If the test antibody is able to bind to ROR2 at the same time as the reference anti-ROR2 antibody, then the test antibody binds to a different epitope than the reference anti-ROR2 antibody. However, if the test antibody is not able to bind to ROR2 at the same time, then the test antibody may bind to the same epitope, an overlapping epitope, or an epitope that is in close proximity to the epitope bound by the anti-ROR2 antibody of the present disclosure. This experiment can be performed using, e.g., ELISA, RIA, Biacore™, SPR, BLI, or flow cytometry. To test whether an anti-ROR2 antibody cross-competes with another anti-ROR2 antibody, one may use the competition method described above in two directions, i.e., determining if the known antibody blocks the test antibody and vice versa. Such cross-competition experiments may be performed, e.g., using a Biacore™ T200, IBIS MX96, or Carterra LSA SPR instrument or the Octet™ system.

The term “antigen-binding portion” or “antigen-binding fragment” of an antibody, as used herein, refers to one or more portions or fragments of an antibody that retain the ability to specifically bind to the antigen (e.g., human ROR2, or a portion thereof) of the antibody. It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, CL and C_{H1} domains; (ii) a F(ab′)₂ fragment: a bivalent fragment comprising two Fab fragments linked by a disulfide bridge at the hinge region; (iii) an Fd fragment consisting of the V_{H} and C_{H1} domains; (iv) an Fv fragment consisting of the V_{L} and V_{H} domains of a single arm of an antibody, (v) a dAb fragment, which consists of a V_{H} domain; and (vi) an isolated CDR capable of specifically binding to an antigen. Furthermore, although the two domains of the Fv fragment, V_{L} and V_{H}, are encoded by separate genes, they can be joined, using recombinant methods, by a synthetic linker that enables them to be made as a single polypeptide chain in which the V_{L} and V_{H} domains pair to form monovalent molecules (known as single chain Fv (scFv)). Also within the present disclosure are antigen-binding molecules comprising a V_{H} and/or a V_{L}. In the case of a V_{H}, the molecule may also comprise one or more of a C_{H1}, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesin molecules can be obtained using standard recombinant DNA techniques, e.g., as described herein.

The class (isotype) and subclass of anti-ROR2 antibodies may be determined by any method known in the art. In general, the class and subclass of an antibody may be determined using antibodies that are specific for a particular class and subclass of antibody. Such antibodies are available commercially. The class and subclass can be determined by ELISA or Western blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies.

In some embodiments, the constant region of the anti-ROR2 antibody herein may comprise mutations that improve the therapeutic potential of the antibody, such as mutations that reduce or eliminate effector functions of the antibody. For example, an antibody may comprise a human IgG1 constant region with the L235E mutation, the P329A mutation, the “LALA” mutations (L234A/L235A), the “LALAPA” mutations (L234A/L235A/P329A), the “LALAGA” mutations (L234A/L235A/G237A), the “LALAGR” mutations (L234A/L235A/G236R), and/or the “LALAPG” mutations (L234A/L235A/P329G) (Eu numbering). Further, for example, the monospecific or multispecific antibody herein may comprise a human IgG4 constant region with the mutation L235E and/or the mutation S228P (Eu numbering). An IgG constant region may comprise mutations that improve the serum half-life of the antibody (e.g., the “YTE” mutation) and/or improve manufacturing and yield of the antibody.

### B. Exemplary Anti-ROR2 Antibodies

An ADC of the invention comprises an antibody or an antigen-binding portion thereof that specifically binds to ROR2. Unless otherwise stated, “ROR2” refers to human ROR2. A human ROR2 polypeptide sequence is available under UniProt Accession No. Q01974 (ROR2_HUMAN), as shown below:

| (SEQ ID NO: 38) | |
|---|---|
| 1 | MARGSALPRR PLICIPAVWA AAALLLSVAR TSGEVEVLDP NDPLGPLDGQ |
| 51 | DGPIPTLKGY FLNFLEPVNN ITIVQGQTAI LHCKVAGNPP PNVRWLKNDA |
| 101 | PVVQEPRRII IRKTEYGSRL RIQDLDTTDT GYYQCVATNG MKTITATGVL |
| 151 | FVRLGPTHSP NHNFQDDYHE DGFCQPYRGI ACARFIGNRT IYVDSLQMQG |
| 201 | EIENRITAAF TMIGTSTHLS DQCSQFAIPS FCHFVFPLCD ARSRTPKPRE |
| 251 | LCRDECEVLE SDLCRQEYTI ARSNPLILMR LQLPKCEALP MPESPDAANC |
| 301 | MRIGIPAERL GRYHQCYNGS GMDYRGTAST TKSGHQCQPW ALQHPHSHHL |
| 351 | SSTDFPELGG GHAYCRNPGG QMEGPWCFTQ NKNVRMELCD VPSCSPRDSS |
| 401 | KMGILYILVP SIAIPLVIAC LFFLVCMCRN KQKASASTPQ RRQLMASPSQ |
| 451 | DMEMPLINQH KQAKLKEISL SAVRFMEELG EDRFGKVYKG HLFGPAPGEQ |
| 501 | TQAVAIKTLK DKAEGPLREE FRHEAMLRAR LQHPNVVCLL GVVTKDQPLS |
| 551 | MIFSYCSHGD LHEFLVMRSP HSDVGSTDDD RTVKSALEPP DFVHLVAQIA |
| 601 | AGMEYLSSHH VVHKDLATRN VLVYDKLNVK ISDIGLFREV YAADYYKLLG |
| 651 | NSLLPIRWMA PEAIMYGKFS IDSDIWSYGV VLWEVFSYGL QPYCGYSNQD |
| 701 | VVEMIRNRQV LPCPDDCPAW VYALMIECWN EFPSRRPREK DIHSRLRAWG |
| 751 | NLSNYNSSAQ TSGASNTTQT SSLSTSPVSN VSNARYVGPK QKAPPFPQPQ |
| 801 | FIPMKGQIRP MVPPPQLYVP VNGYQPVPAY GAYLPNFYPV QIPMQMAPQQ |
| 851 | VPPQMVPKPS SHHSGSGSTS TGYVTTAPSN TSMADRAALL SEGADDTQNA |
| 901 | PEDGAQSTVQ EAEEEEEGSV PETELLGDCD TLQVDEAQVQ LEA |

In the above sequence, the extracellular domain spans amino acids 34-403. The anti-ROR2 antibodies herein bind to an epitope in the extracellular domain.

Amino acid sequences of exemplary anti-ROR2 antibodies used in the ADCs of the present disclosure are shown in Table 1 below.

TABLE 1
SEQ ID NOs of Exemplary Anti-ROR2 Antibodies

| Ab | HC | LC | VH | VL | HCDR1 | HCDR2 | HCDR3 | LCDR1 | LCDR2 | LCDR3 |
|---|---|---|---|---|---|---|---|---|---|---|
| 1 | 1 | 2 | 3 | 4 | 5 | 6 | 7 | 8 | 9 | 10 |
| 2 | 11 | 2 | 12 | 4 | 13 | 6 | 14 | 8 | 9 | 10 |
| 3 | 15 | 16 | 17 | 18 | 19 | 6 | 20 | 8 | 9 | 10 |
| 4 | 21 | 16 | 17 | 18 | 19 | 6 | 20 | 8 | 9 | 10 |

Ab1 and Ab2 are humanized, effectorless versions of murine anti-ROR2 antibody 6E6 (see, e.g., PCT Patent Publication WO 2021/102055 and PCT Patent Application PCT/US2023/086552). Ab3 is a chimeric version of 6E6. Ab4 is a chimeric, effectorless version of 6E6.

In some embodiments, an anti-ROR2 antibody or antigen-binding portion thereof herein competes or cross-competes for binding to human ROR2 with, or binds to the same epitope of human ROR2 as, an antibody comprising:
a) a heavy chain (HC) comprising the amino acid sequence of SEQ ID NO: 1 and a light chain (LC) comprising the amino acid sequence of SEQ ID NO: 2;
b) an HC comprising the amino acid sequence of SEQ ID NO: 11 and an LC comprising the amino acid sequence of SEQ ID NO: 2;
c) an HC comprising the amino acid sequence of SEQ ID NO: 15 and an LC comprising the amino acid sequence of SEQ ID NO: 16; or
d) an HC comprising the amino acid sequence of SEQ ID NO: 21 and an LC comprising the amino acid sequence of SEQ ID NO: 16.

In some embodiments, the anti-ROR2 antibody or antigen-binding portion has HCDR1-3 comprising the amino acid sequences of
SEQ ID NOs: 5, 6, and 7, respectively;
SEQ ID NOs: 13, 6, and 14, respectively; or
SEQ ID NOs: 19, 6, and 20, respectively.

In some embodiments, the anti-ROR2 antibody or antigen-binding portion has a heavy chain variable domain (V_{H}) amino acid sequence that is at least 80%, 85%, 90%, 91%, 92%, 93%, 94%, 95%, 96%, 97%, 98%, or 99% identical to the amino acid sequence of SEQ ID NO: 3, 12, or 17.

In some embodiments, the anti-ROR2 antibody or antigen-binding portion has a V_{H} comprising the amino acid sequence of SEQ ID NO: 3, 12, or 17.

In some embodiments, the anti-ROR2 antibody has an HC amino acid sequence that is at least 80%, 85%, 90%, 91%, 92%, 93%, 94%, 95%, 96%, 97%, 98%, or 99% identical to the amino acid sequence of SEQ ID NO: 1, 11, 15, or 21, or said sequence without the C-terminal lysine.

In some embodiments, the anti-ROR2 antibody comprises an HC amino acid sequence of SEQ ID NO: 1, 11, 15, or 21, or said sequence without the C-terminal lysine.

In some embodiments, the anti-ROR2 antibody or antigen-binding portion has LCDR1-3 comprising the amino acid sequences of SEQ ID NOs: 8-10, respectively.

In some embodiments, the anti-ROR2 antibody or antigen-binding portion has a light chain variable domain (V_{L}) amino acid sequence that is at least 80%, 85%, 90%, 91%, 92%, 93%, 94%, 95%, 96%, 97%, 98%, or 99% identical to the amino acid sequence of SEQ ID NO: 4 or 18.

In some embodiments, the anti-ROR2 antibody or antigen-binding portion has a V_{L} comprising the amino acid sequence of SEQ ID NO: 4 or 18.

In some embodiments, the anti-ROR2 antibody has an LC amino acid sequence that is at least 80%, 85%, 90%, 91%, 92%, 93%, 94%, 95%, 96%, 97%, 98%, or 99% identical to the amino acid sequence of SEQ ID NO: 2 or 16.

In some embodiments, the anti-ROR2 antibody comprises an LC amino acid sequence of SEQ ID NO: 2 or 16.

In certain embodiments, the anti-ROR2 antibody or antigen-binding portion comprises any of the above-described heavy chain sequences paired with any one of the above-described light chain sequences.

In some embodiments, the anti-ROR2 antibody or antigen-binding portion herein comprises the HCDR1-3 and LCDR1-3 amino acid sequences of:
a) SEQ ID NOs: 5, 6, 7, 8, 9, and 10, respectively;
b) SEQ ID NOs: 13, 6, 14, 8, 9, and 10, respectively; or
c) SEQ ID NOs: 19, 6, 20, 8, 9, and 10, respectively.

In some embodiments, the anti-ROR2 antibody or antigen-binding portion herein comprises a V_{H} and a V_{L} that are at least 80%, 85%, 90%, 91%, 92%, 93%, 94%, 95%, 96%, 97%, 98%, or 99% identical (e.g., at least 90% identical) to the amino acid sequences of:
a) SEQ ID NOs: 3 and 4, respectively;
b) SEQ ID NOs: 12 and 4, respectively; or
c) SEQ ID NOs: 17 and 18, respectively.

In some embodiments, the anti-ROR2 antibody or antigen-binding portion herein comprises a V_{H} and a V_{L} that comprise the amino acid sequences of:
a) SEQ ID NOs: 3 and 4, respectively;
b) SEQ ID NOs: 12 and 4, respectively; or
c) SEQ ID NOs: 17 and 18, respectively.

In some embodiments, the anti-ROR2 antibody herein comprises an HC and an LC that comprise the amino acid sequences of:
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively;
optionally wherein the HC amino acid sequence is without the C-terminal lysine.

The anti-ROR2 antibody or antigen-binding portion herein may compete or cross-compete for binding to human ROR2 with, or bind to the same epitope of human ROR2 as, any one of Ab1-Ab4.

In some embodiments, the anti-ROR2 antibody or antigen-binding portion comprises
HCDR1 comprising SEQ ID NO: 40;
HCDR2 comprising SEQ ID NO: 28; and
HCDR3 comprising SEQ ID NO: 24;
and/or
LCDR1 comprising SEQ ID NO: 41;
LCDR2 comprising SEQ ID NO: 9; and
LCDR3 comprising SEQ ID NO: 37.

In some embodiments, the anti-ROR2 antibody or antigen-binding portion herein comprises the HCDR1-3 and LCDR1-3 amino acid sequences of any one of Ab1-Ab4. The assignment of CDR regions may be in accordance with any method known in the art, such as IMGT®, Kabat, Chothia, Martin, Contact, or AHo definitions, or any combination of any of these definitions (Kabat plus Chothia, for example). Examples of CDR definitions under different methods is shown below for Ab1 (SEQ: SEQ ID NO):

Ab1 HCDRs

| Definition | HCDR1 | SEQ | HCDR2 | SEQ | HCDR3 | SEQ |
|---|---|---|---|---|---|---|
| IMGT ® | GFTFSTYG | 5 | ISSGGGYT | 6 | ARHPRDFSYALDY | 7 |
| Kabat | TYGVS | 22 | TISSGGGYTHYAGSVKG | 23 | HPRDFSYALDY | 24 |
| Chothia | GFTFSTY | 27 | SSGGGY | 28 | HPRDFSYALDY | 24 |
| AHo | AASGFTFSTYGVS | 29 | TISSGGGYTH | 30 | ARHPRDFSYALDY | 7 |
| Contact | STYGVS | 32 | WVSTISSGGGYTH | 33 | ARHPRDFSYALD | 34 |

Ab1 LCDRs

| Definition | LCDR1 | SEQ | LCDR2 | SEQ | LCDR3 | SEQ |
|---|---|---|---|---|---|---|
| IMGT ® | QDVGHY | 8 | WAS | 9 | QQYNIYPWT | 10 |
| Kabat | RASQDVGHYLA | 25 | WASTRAT | 26 | QQYNIYPWT | 10 |
| Chothia | RASQDVGHYLA | 25 | WASTRAT | 26 | QQYNIYPWT | 10 |
| AHo | RASQDVGHYLA | 25 | YWASTRAT | 31 | QQYNIYPWT | 10 |
| Contact | GHYLAWY | 35 | LLIYWASTRA | 36 | QQYNIYPW | 37 |

Thus, for example, the Ab1 IMGT®-defined HCDR1-3 and LCDR1-3 sequences of SEQ ID NOs: 5-10, respectively, may be replaced in any embodiment described herein by
SEQ ID NOs: 22, 23, 24, 25, 26, and 10, respectively;
SEQ ID NOs: 27, 28, 24, 25, 26, and 10, respectively;
SEQ ID NOs: 29, 30, 7, 25, 31, and 10, respectively; or
SEQ ID NOs: 32, 33, 34, 35, 36, and 37, respectively.
   Also contemplated is a set of Ab1 CDRs wherein each of HCDR1, HCDR2, HCDR3, LCDR1, LCDR2, and LCDR3 may individually be specified according to any of the methods for defining Ab1 CDRs as shown above (e.g., HCDR1 specified by the Kabat definition, HCDR2 specified by the Chothia definition, etc.). For example, in some embodiments, the anti-ROR2 antibody or antigen-binding portion thereof comprises:
an HCDR1 comprising SEQ ID NO: 5, 22, 27, 29, or 32;
an HCDR2 comprising SEQ ID NO: 6, 23, 28, 30, or 33;
an HCDR3 comprising SEQ ID NO: 7, 24, or 34;
an LCDR1 comprising SEQ ID NO: 8, 25, or 35;
an LCDR2 comprising SEQ ID NO: 9, 26, 31, or 36; and/or
an LCDR3 comprising SEQ ID NO: 10 or 37.

The same means for defining Ab1 CDRs are contemplated for any of Ab2-Ab4.

In some embodiments, the anti-ROR2 antibody or antigen-binding portion herein comprises a V_{H} and a V_{L} that are at least 80%, 85%, 90%, 91%, 92%, 93%, 94%, 95%, 96%, 97%, 98%, or 99% identical in amino acid sequence to the V_{H} and V_{L}, respectively, of any one of Ab1-Ab4.

In some embodiments, the anti-ROR2 antibody or antigen-binding portion herein comprises a V_{H} and a V_{L} that are the V_{H} and V_{L}, respectively, of any one of Ab1-Ab4.

In some embodiments, the anti-ROR2 antibody is any one of Ab1-Ab4, or an antibody with the same amino acid sequences as said antibody.

In some embodiments, the anti-ROR2 antibody or antigen-binding portion is a variant antibody or antigen-binding portion. A “variant” antibody or antigen-binding portion has amino acid substitutions (which may be conservative or non-conservative) from a reference antibody or antigen-binding portion but does not have substantially altered biologic activity as compared to the reference antibody or antigen-binding portion. For example, the variant antibody or antigen-binding portion may retain at least 50%, 70%, 75%, 80%, 85%, 90%, 95%, or 100% of the binding affinity of the reference antibody or antigen-binding portion, or may exceed the binding affinity of the reference antibody or antigen-binding portion. In some embodiments, a variant antibody or an antigen-binding portion thereof may have mutations, e.g., that increase its half-life, alter its immunogenicity, provide a site for covalent or non-covalent binding to another molecule, etc. In certain embodiments, the variant antibody or antigen-binding portion thereof may have mutations in its FRs (e.g., in one, two, three, four, five, six, seven, or eight of its FRs). In certain embodiments, the variant antibody or antigen-binding portion thereof may have mutations in its CDRs (e.g., in one, two, three, four, five, or six of its CDRs). In certain embodiments, the variant antibody or antigen-binding portion thereof may have mutations in its constant regions.

The class of an anti-ROR2 antibody described herein may be changed or switched with another class or subclass. For example, an anti-ROR2 antibody that was originally IgM may be class switched to IgG. Further, the class switching may be used to convert one IgG subclass to another, e.g., from IgG₁ to IgG₂. A κ light chain constant region can be changed, e.g., to a λ light chain constant region, or vice versa.

The anti-ROR2 antibody herein can be an IgG, an IgM, an IgE, an IgA, or an IgD molecule, but is typically of the IgG isotype, e.g., of IgG subclass IgG₁, IgG2a or IgG2b, IgG3 or IgG4.

In some embodiments, the anti-ROR2 antibody may comprise at least one mutation in the Fc region. A number of different Fc mutations are known, where these mutations alter, e.g., the antibody's effector functions or half-life. For example, in some embodiments, the anti-ROR2 antibody comprises at least one mutation in the Fc region that reduces or eliminates effector function. In certain embodiments, the anti-ROR2 antibody may comprise, e.g., L234A, L235A, and/or P329A mutations (Eu numbering), wherein the mutations may appear alone or in any combination. In particular embodiments, the anti-ROR2 antibody may comprise an Fc region with all three mutations.

### C. Cytotoxic Drug Moieties

An ADC of the invention comprises an anti-ROR2 antibody or an antigen-binding portion thereof conjugated to one or more drug moieties, for example, cytotoxic drug moieties such as chemotherapeutic agents, growth inhibitory agents, toxins (e.g., protein toxins, enzymatically active toxins of bacterial, fungal, plant, or animal origin, or fragments thereof), or radioactive isotopes. The cytotoxic drug moiety can be conjugated to the anti-ROR2 antibody or antigen-binding portion by a linker covalently bound to an amino acid residue (e.g., a cysteine or a lysine) of the antibody or antigen-binding portion. Many drugs that can serve as a cytotoxic moiety in an immunoconjugate are independently too toxic to be used for cancer treatment and thus are more effective and safer when specifically targeted to the cancer cell by an antibody or antigen-binding portion thereof.

The term “cytotoxic drug moiety” or “cytotoxic agent” refers to a compound that can cause harm, disturbance, or death to a cell. Examples of cytotoxic drug moieties that can be used as part of an anti-ROR2 immunoconjugate herein include, but are not limited to: NCA1, auristatin, auristatin E, DNA minor groove binding agents, DNA minor groove alkylating agents, enediyne, lexitropsin, duocarmycin, taxane, puromycin, dolastatin, maytansinoid, vinca alkaloid, AFP, MMAF, MMAE, AEB, AEVB, taxoids (e.g., paclitaxel and paclitaxel derivatives (TAXOL®, Bristol-Myers Squibb Oncology, Princeton, N.J.), ABRAXANE® (American Pharmaceutical Partners, Schaumberg, Ill.), as well as docetaxel and docetaxel derivatives), CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, netropsin, podophyllotoxin (e.g., etoposide and teniposide), baccatin and its derivatives, anti-tubulin agents, cryptophysin, combretastatin, vincristine, vincristine sulfate, vinblastine, vindesine, vinorelbine, VP-16, camptothecin, epothilone A, epothilone B, nocodazole, colchicines, colcimid, estramustine, cemadotin, discodermolide, eleutherobin, mechlorethamine, cyclophosphamide, melphalan, carmustine, lomustine, semustine, streptozocin, chlorozotocin, uracil mustard, chlormethine, chlorambucil, pipobroman, triethylenemelamine, triethylenethiophosphoramine, busulfan, dacarbazine, temozolomide, ytarabine, cytosine arabinoside, fluorouracil, 5-fluorouracil (5-FU), floxuridine, 6-thioguanine, 6-mercaptopurine, pentostatin, methotrexate, 10-propargyl-5,8-dideazafolate, 5,8-dideazatetrahydrofolic acid, leucovorin, fludarabine phosphate, pentostatine, gemcitabine, Ara-C, deoxycoformycin, mitomycins such as mitomycin-C, L-asparaginase, azathioprine, brequinar, antibiotics (e.g., anthracycline, gentamicin, cefalotin, vancomycin, telavancin, daptomycin, azithromycin, erythromycin, rocithromycin, furazolidone, amoxicillin, ampicillin, carbenicillin, flucloxacillin, methicillin, penicillin, ciprofloxacin, moxifloxacin, ofloxacin, doxycycline, minocycline, oxytetracycline, tetracycline, streptomycin, rifabutin, ethambutol, and rifaximin), enediyne antibiotics (e.g., calicheamicin, calicheamicin gamma1I and calicheamicin omegaI1, and dynemicin, including dynemicin A), antiviral drugs (e.g., abacavir, acyclovir, ampligen, cidofovir, delavirdine, didanosine, efavirenz, entecavir, fosfonet, ganciclovir, ibacitabine, immunovir, idoxuridine, inosine, lopinavir, methisazone, nexavir, nevirapine, oseltamivir, penciclovir, stavudine, trifluridine, truvada, valaciclovir, and zanamivir), daunorubicin hydrochloride, daunoriycin, rubidomycin, cerubidine, idarubicin, doxorubicin, epirubicin and morpholino derivatives, phenoxizone biscyclopeptides (e.g., dactinomycin), basic glycopeptides (e.g., bleomycin), anthraquinone glycosides (e.g., plicamycin and mithramycin), anthracenediones (e.g., mitoxantrone), azirinopyrrolo indolediones (e.g., mitomycin), macrocyclic immunosuppressants (e.g., cyclosporine, FK-506, tacrolimus, prograf, and rapamycin), navelbene, CPT-11, anastrazole, letrazole, capecitabine, reloxafine, droloxafine, allocolchicine, Halichondrin B, colchicine and colchicine derivatives, rhizoxin, thiocolchicine, trityl cysterin, vinblastine sulfate, hydroxyurea, N-methylhydrazine, epidophyllotoxin, procarbazine, mitoxantrone, leucovorin, and tegafur. “Taxanes” include paclitaxel, as well as any active taxane derivative or pro-drug. Chemotherapeutic agents include erlotinib (TARCEVA®, Genentech/OSI Pharm.), bortezomib (VELCADE®, Millenium Pharm.), fulvestrant (FASLODEX®, AstraZeneca), sunitinib (Sutent®, Pfizer), letrozole (FEMARA®, Novartis), imatinib mesylate (GLEEVEC®, Novartis), PTK787/ZK 222584 (Novartis), oxaliplatin (Eloxatin®, Sanofi), leucovorin, lapatinib (TYKERB®, GSK572016, GlaxoSmithKline), lonafarnib (SCH 66336), sorafenib (BAY43-9006, Bayer Labs.), and gefitinib (IRESSA®, AstraZeneca), AG1478, AG1571 (SU 5271; Sugen), alkylating agents such as thiotepa and cyclosphosphamide (CYTOXAN®); alkyl sulfonates such as busulfan, improsulfan and piposulfan; antifolate antineoplastic such as pemetrexed (ALIMTA®, Eli Lilly); aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (such as bullatacin and bullatacinone); a camptothecin (including the synthetic analogue topotecan); bryostatin; callystatin; cryptophycins (such as cryptophycin 1 and cryptophycin 8); dolastatin; duocarmycin (including its synthetic analogues KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, fotemustine, lomustine, nimustine, and ranimnustine; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (ADRIAMYCIN®) (including morpholino-doxorubicin, cyanomorpholino-doxorubicin, 2-pyrrolino-doxorubicin and deoxydoxorubicin), epirubicin, esorubicin, idarubicin, marcellomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; a folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE™ Cremophor-free, albumin, nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); mitoxantrone; NAVELBINE® vinorelbine; novantrone; teniposide; edatrexate; daunomycin; aminopterin; xeloda; ibandronate; topoisomerase inhibitor RFS 2000; difluoromethylornithine (DMFO); retinoids such as retinoic acid; and pharmaceutically acceptable salts, esters, acids, prodrugs, or derivatives of any of the above.

In some embodiments, the drug moiety may promote protein degradation (“degrader”). For example, in certain embodiments, the drug moiety may be a heterobifunctional small molecule that recruits a ubiquitin ligase (e.g., an E3 ubiquitin ligase such as CRBN, VHL, or XIAP) to cause the degradation of a specific target protein. The drug moiety may be a proteolysis targeting chimera (PROTAC). An antibody or an antigen-binding portion thereof herein thus may be linked to a heterobifunctional moiety that causes the degradation of a protein, such as AR, BCL-XL, BRD9, IRAK4, STAT3, BTK, BET, BRD4r, ERα, TGFβR2, BRM, or GSTP1.

In some embodiments, a suitable cytotoxic drug moiety for use in an anti-ROR2 ADC of the present disclosure is
a) an anti-tubulin agent (e.g., an auristatin or dolastatin such as auristatin E, monomethyl auristatin E (MMAE) or monomethyl auristatin F (MMAF)),
b) a DNA alkylating agent or a DNA minor groove alkylating agent (e.g., duocarmycin or a duocarmycin derivative), or
c) a topoisomerase I inhibitor (e.g., exatecan, an exatecan derivative (DXd), or SN-38).
   In some embodiments, the cytotoxic agent used in the anti-ROR2 ADC is selected from the group consisting of DXd, exatecan, MMAE, MMAF, and SN-38. In some embodiments, the drug moiety D comprises exatecan.

In some embodiments, the cytotoxic drug moiety in the immunoconjugate is a prodrug. The term “prodrug” or “pharmaceutically acceptable prodrug,” as used herein, refers to an agent that is converted into the parent drug in vivo or inside a cell and is relatively nontoxic. An immunoconjugate conjugated to a prodrug may cause fewer undesirable biological effects or may be administered without interacting in a deleterious manner with any of the components of the composition in which it is contained. Prodrugs are generally drug precursors (which may be a pharmacologically inactive or less active derivative of the drug) that, following administration to a subject and subsequent absorption, are converted to an active or a more active species via some process, such as conversion by a metabolic pathway. The process may be enzymatic or non-enzymatic. Some prodrugs have a chemical group present on the prodrug that renders it less active and/or confers solubility or some other property to the drug. Once the chemical group has been cleaved and/or modified from the prodrug, the active drug is generated. Prodrugs may provide improved physiochemical properties over their parent drugs such as better solubility, enhanced delivery characteristics (e.g., targeting a particular cell, tissue, organ or ligand), or improved therapeutic value of the drug. The benefits of such prodrugs may include, but are not limited to, (i) ease of administration compared with the parent drug; (ii) increased bioavailability of the prodrug immunoconjugate as compared to the parent drug immunoconjugate; and (iii) immunoconjugates comprising the prodrug may have improved solubility in pharmaceutical compositions compared with the parent drug. Prodrugs may be designed to modulate the amount of a drug or biologically active molecule that reaches a desired site of action through the manipulation of the properties of a drug, such as physiochemical, biopharmaceutical, or pharmacokinetic properties.

In some embodiments, the average number of the drug moiety to the antibody in the immunoconjugate (i.e., drug-to-antibody ratio or DAR) is 1; or at least 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 6, 7, 8, 9, or 10. In some embodiments, the DAR is between 3 and 8. For example, in certain embodiments, the DAR is 3.2, 3.5, 4, 4.3, 7, 7.1, 7.5, 7.6, 7.8, 7.9, or 8.

### D. Linkers

In certain embodiments of an anti-ROR2 ADC described herein, the antibody or antigen-binding portion can be conjugated directly to the cytotoxic agent, or can be conjugated via a linker. Suitable linkers include, for example, cleavable (e.g., either enzymatically or chemically) and non-cleavable linkers. In some embodiments, the linker is a cleavable linker. A cleavable linker refers to a linker that comprises a cleavable moiety that is typically susceptible to cleavage under intracellular conditions or tumor microenvironment conditions (extracellular). Suitable cleavable linkers include, for example, peptide linkers cleavable by an intracellular protease (such as a lysosomal protease or an endosomal protease) or extracellular protease (e.g., secreted lysosomal or endosomal protease, metalloprotease, serine protease, cysteine protease, aspartic protease, threonine protease, etc.) and acid-cleavable linkers. Other suitable cleavable linkers include acid triggers (e.g., hydrazone, carbonate, silyl ether, etc.), glutathione triggers (e.g., disulfide), iron (II) triggers (e.g., 1,2,4-trioxolane), glycosidase triggers (e.g., β-glucuronide, β-galactoside, etc.), phosphatase triggers (e.g., pyrophosphate), and sulfatase triggers (e.g., arylsulfate).

In some embodiments, the linker can be or comprise an amino acid moiety, such as lysine (Lys or K) or phenylalanine (Phe or F), a dipeptide moiety, such as valine-citrulline (Val-Cit or VC) or valine-alanine (Val-Ala or VA), a tripeptide moiety, such as glycine-valine-citrulline (Gly-Val-Cit or GVC), glycine-glycine-glycine (Gly-Gly-Gly or GGG), or alanine-alanine-asparagine (Ala-Ala-Asn or AAN), or a tetrapeptide moiety, such as glycine-glycine-phenylalanine-glycine (Gly-Gly-Phe-Gly or GGFG (SEQ ID NO: 39)). The linker may also be a pentapeptide or a polypeptide having six or more amino acid residues. In some embodiments, the linker can be a peptide moiety rich in proline, alanine, and/or serine. In some embodiments, the peptide moiety can be linked to an alkyl group (e.g., a C₃ or C₅ alkyl group) or polyethylene glycol (e.g., PEG2). In some embodiments, the peptide moiety can be an enzymatically cleavable peptide moiety. In other embodiments, the peptide moiety can be a non-cleavable peptide moiety.

In some embodiments, the linker can be an acid cleavable linker. In some embodiments, the acid cleavable linker may comprise a hydrazone linkage, which may be susceptible to hydrolytic cleavage at a pH of less than (7, e.g., less than 5.5). In some embodiments the acid cleavable linker may comprise a thiomaleamic acid linker. In some embodiments, the acid cleavable linker may be a thiomaleamic acid linker as described in Castaneda et al., Chem Commun. (2013) 49:8187-9.

Additional suitable cleavable linkers include disulfide linkers. In some embodiments, the linker may be a non-polymeric linker. In some cases, the linker may be a non-peptide linker or a linker that does not contain an amino acid residue.

In some embodiments, the linker includes a C₁-C₆ alkyl group (e.g., a C₅, C₄, C₃, C₂, or C₁ alkyl group). As used herein the term “alkyl” refers to a straight or branched hydrocarbon chain radical consisting solely of carbon and hydrogen atoms, containing no unsaturation. C₁-Cₓ includes C₁-C₂, C₁-C₃, . . . , C₁-Cₓ, where x is an integer. C₁-Cₓ refers to the number of carbon atoms in the designated group. In some embodiments, the alkyl comprises one to eight carbon atoms (C₁₋₈ alkyl). In some embodiments, the alkyl comprises three to five carbon atoms (C₃₋₅ alkyl). In particular embodiments, the alkyl is a C₃ or C₅ alkyl. In some embodiments, the linker comprises an arylene (e.g., phenylene) group. As used herein, the term “arylene” refers to a disubstituted aromatic ring and the term “phenylene” refers to a disubstituted six-carbon aromatic ring, i.e., a disubstituted benzene ring.

In some embodiments, the linker includes a polyalkylene glycol (e.g., polyethylene glycol or PEG) moiety. In some embodiments, the linker includes a PEGx moiety wherein x is an integer between 1 and 20. In some embodiments, the linker includes a polypeptoid (e.g., a polysarcosine or pSAR) moiety. In some embodiments, the linker includes a pSARx moiety wherein x is an integer between 1 and 20. In some embodiments, the linker includes one or more polyhydroxyl and/or polycarboxyl groups.

In some embodiments, the linker is a self-immolative linker or a self-elimination linker (e.g., a cyclization self-elimination linker) comprising one or more self-immolative spacer moieties. Exemplary self-immolative spacer moieties include an amino methylene (—NH₂CH₂— or AM) spacer, a p-aminobenzyloxycarbonyl (PAB) spacer, and a N,N′-dimethylethylenediamine (DMEDA) spacer. In some embodiments, the self-immolative linker may be a linker described in U.S. Pat. No. 9,089,614 or PCT Publication WO 2015/038426.

In some embodiments, the linker is a dendritic type linker. In certain embodiments, the dendritic type linker comprises a branching, multifunctional linker moiety. The dendritic linker can have two or more branches. In certain embodiments, the dendritic type linker is used to increase the molar ratio of the drug moiety to the antibody or antigen-binding portion thereof. In certain embodiments, the dendritic type linker comprises PAMAM dendrimers.

In some embodiments, the linker is a traceless linker or a linker which after cleavage does not leave behind a linker moiety (e.g., an atom or a linker group). Exemplary traceless linkers include, but are not limited to, germanium linkers, silicium linkers, sulfur linkers, selenium linkers, nitrogen linkers, phosphorus linkers, boron linkers, chromium linkers, and phenylhydrazide linkers. In some embodiments, the linker is a traceless aryl-triazene linker as described in Hejesen et al., Org Biomol Chem. (2013) 11(15):2493-7. In some embodiments, the linker is a traceless linker described in Blaney et al., Chem Rev. (2002) 102:2607-24. In some embodiments, a linker is a traceless linker as described in U.S. Pat. No. 6,821,783.

In some embodiments, the linker comprises a functional group that exerts steric hindrance at the site of bonding between the linker and a conjugating moiety. In some embodiments, the steric hindrance is a steric hindrance around a disulfide bond. An exemplary linker that exhibits steric hindrance may be, e.g., a heterobifunctional linker (e.g., as described herein). In some embodiments, a linker that exhibits steric hindrance comprises SMCC and SPDB.

As used herein in the context of the linker, valine-citrulline (Val-Cit or VC) refers to a linker moiety having the structure:

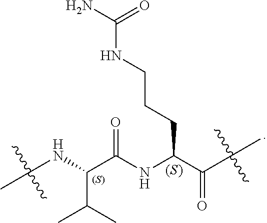

As used herein in the context of the linker, valine-alanine (Val-Ala or VA) refers to a linker moiety having the structure:

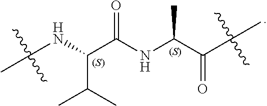

As used herein in the context of the linker, glycine-valine-citrulline (Gly-Val-Cit or GVC) refers to a linker moiety having the structure:

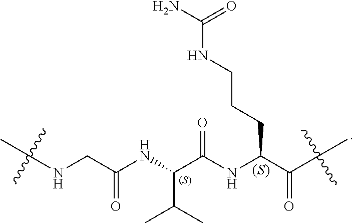

As used herein in the context of the linker, glycine-glycine-glycine (Gly-Gly-Gly or GGG) refers to a linker moiety having the structure:

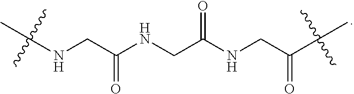 [SMILES: *NCC(=O)NCC(=O)NCC(*)=O]

As used herein in the context of the linker, alanine-alanine-asparagine (Ala-Ala-Asn or AAN), refers to a linker moiety having the structure:

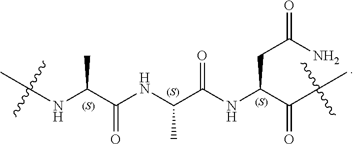

As used herein in the context of the linker, glycine-glycine-phenylalanine-glycine (Gly-Gly-Phe-Gly or GGFG (SEQ ID NO: 39) refers to a linker moiety having the structure:

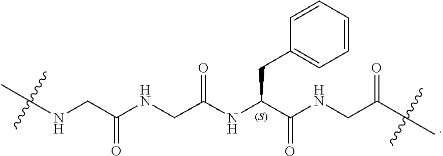

As used herein in the context of the linker, C₂ refers to a linker moiety having the structure:

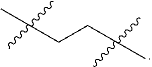 [SMILES: *CC*]

As used herein in the context of the linker, C₃ refers to a linker moiety having the structure:

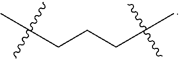 [SMILES: *CCC*]

As used herein in the context of the linker, C₅ refers to a linker moiety having the structure:

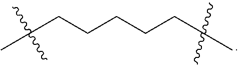 [SMILES: *CCCCC*]

As used herein in the context of the linker, PEG2 refers to a linker moiety having the structure:

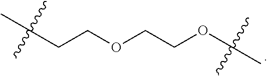 [SMILES: *CCOCCO*]

As used herein in the context of the linker, para-aminobenzyloxycarbonyl (PAB) refers to a linker moiety having the structure:

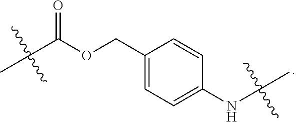 [SMILES: *Nc1ccc(COC(*)=O)cc1]

As used herein in the context of the linker, phenylene (Ph) may refer to a linker moiety having the structure:

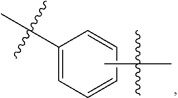

wherein the substitutions are ortho, para, or meta to each other. In some embodiments, the phenylene is para substituted (Ph(p)) as shown by the structure:

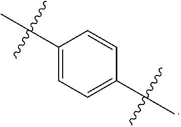 [SMILES: *c1ccc(*)cc1]

In some embodiments, the phenylene is meta substituted (Ph(m)) as shown by the structure:

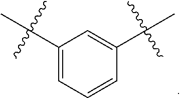 [SMILES: *c1cccc(*)c1]

As used herein in the context of the linker, amino methylene (AM) refers to a linker moiety having the structure:

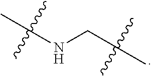 [SMILES: *CN*]

As used herein in the context of the linker, PAS10 refers to a linker moiety having the structure:

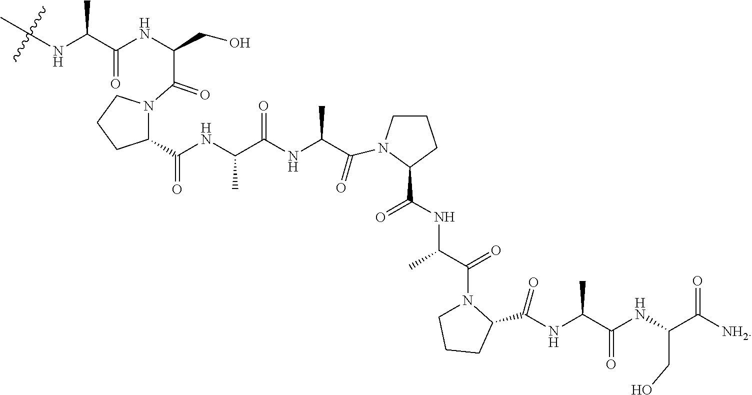 [SMILES: *N[C@@H](C)C(=O)N[C@@H](CO)C(=O)N1CCC[C@H]1C(=O)N[C@@H](C)C(=O)N[C@@H](C)C(=O)N1CCC[C@H]1C(=O)N[C@@H](C)C(=O)N1CCC[C@H]1C(=O)N[C@@H](C)C(=O)N[C@@H](CO)C(N)=O]

As used herein in the context of the linker, Gly-Sar refers to a linker moiety having the structure:

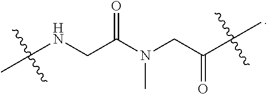 [SMILES: *NCC(=O)N(C)CC(*)=O]

In some embodiments, the present ADC comprises a Core 1-Type (CR1) Linker, and may be represented by Formula (I), Formula (Ia), Formula (Ib), Formula (Ic), Formula (Id), Formula (Ie), or Formula (If), or a pharmaceutically acceptable salt, or if appropriate, ester, thereof (DAR not illustrated; that is, each antibody or antigen-binding portion thereof may have more than one linker/payload unit):

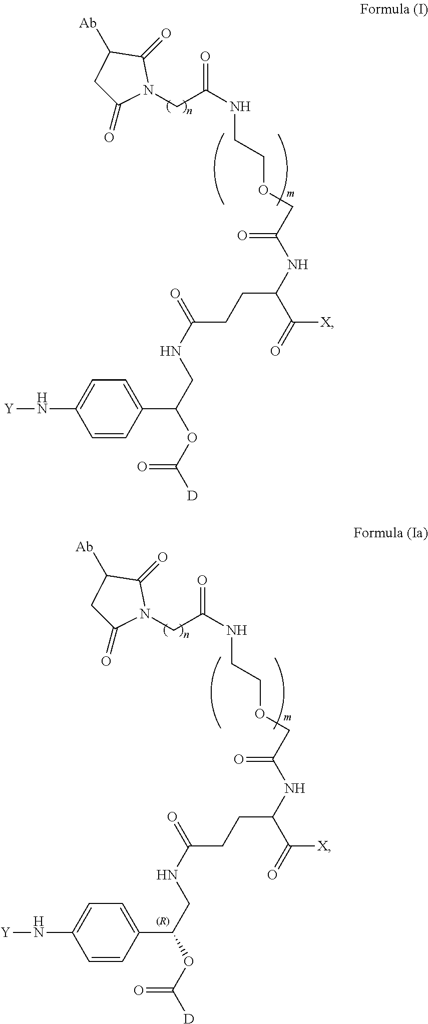

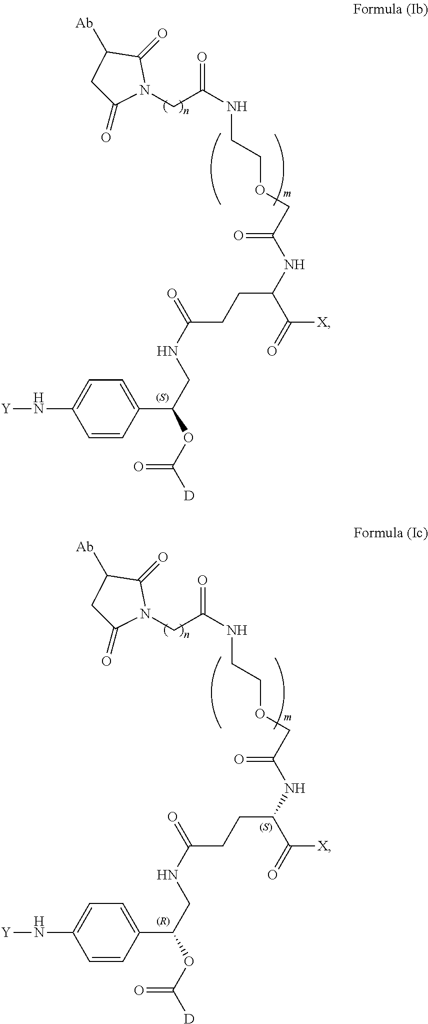

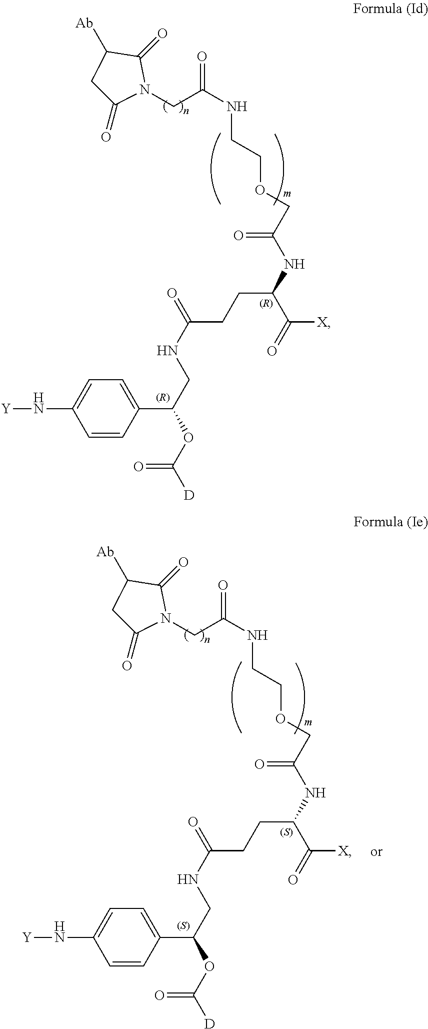

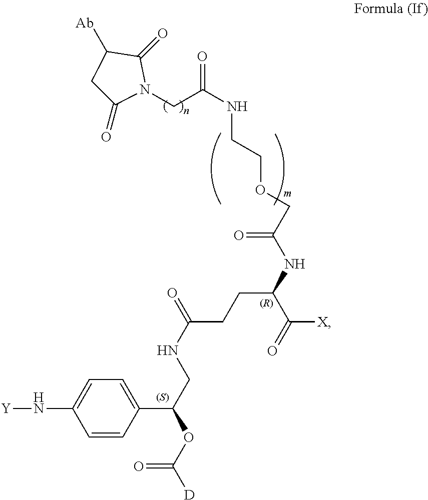

wherein
Ab is an anti-ROR2 antibody or an antigen-binding portion thereof,
n is 0-10,
m is 0-5,
D is a drug moiety,
X is a peptide or hydrophilic moiety, and
Y is a cleavable peptide or β-glucuronide-containing moiety.

In some embodiments, n is 2 and m is 2.

In some embodiments, Y is a cleavable dipeptide moiety, such as a valine-alanine dipeptide, optionally wherein the terminal amino group of the dipeptide moiety, such as the terminal amino group of the valine residue in the valine-alanine dipeptide, is acylated.

In some embodiments, the present ADC comprising the CR1 linker may be represented by Formula (II), Formula (IIa), Formula (IIb), Formula (IIc), Formula (IId), Formula (IIe), or Formula (IIf), or a pharmaceutically acceptable salt or if appropriate, ester, thereof (DAR not illustrated):

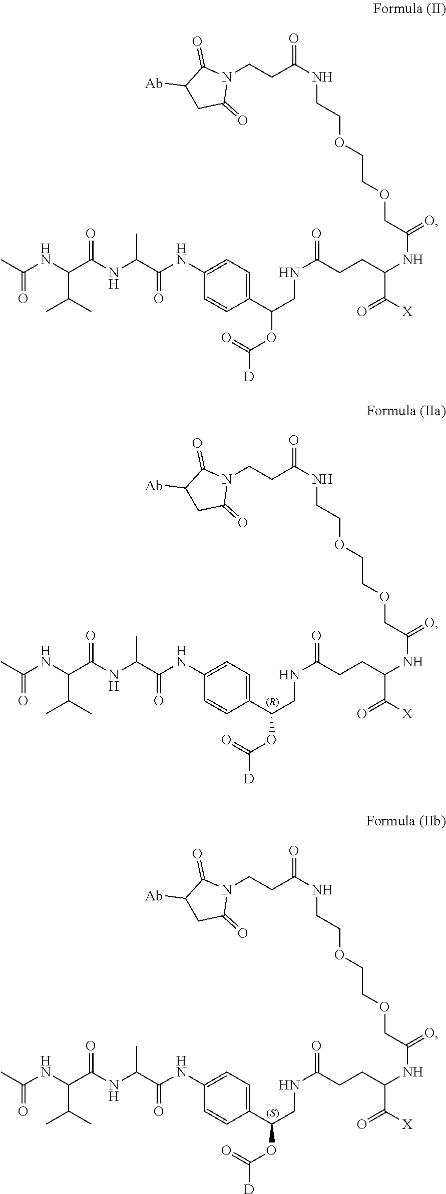

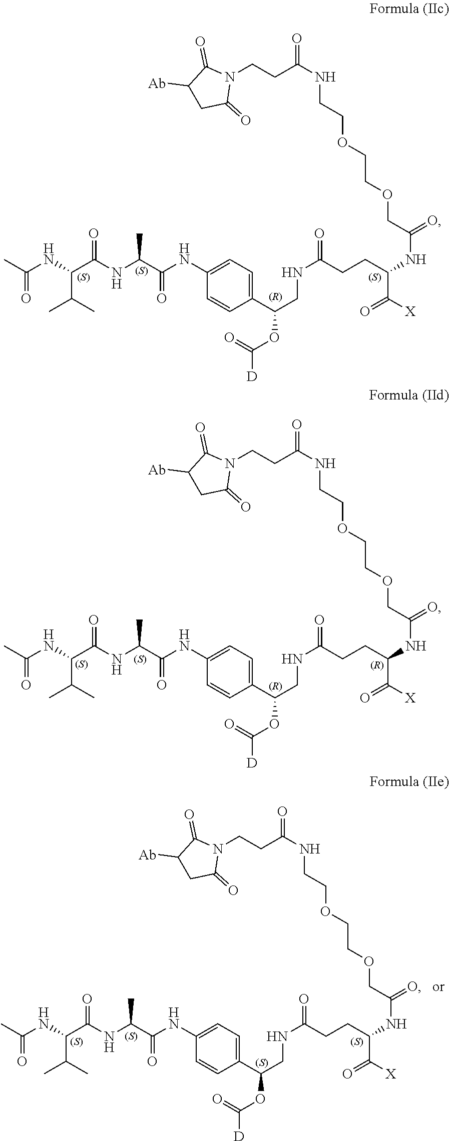

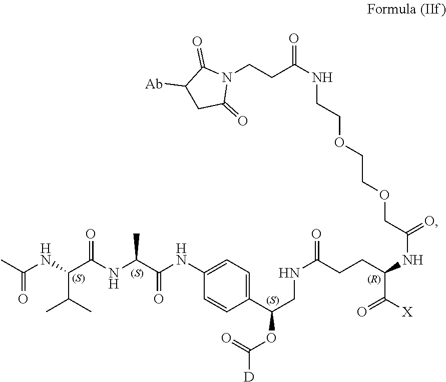

wherein D is a drug moiety and X is a peptide or hydrophilic moiety.

In some embodiments, X is a peptide moiety comprising 1 to 50 amino acid residues. In some embodiments, the amino acid residues of X are selected from the group consisting of alanine, arginine, asparagine, aspartic acid, cysteine, glutamic acid, glutamine, glycine, histidine, isoleucine, leucine, lysine, methionine, phenylalanine, proline, sarcosine, serine, threonine, tryptophan, tyrosine, valine, citrulline, and β-alanine. In certain embodiments, the amino acids of X are selected from the group consisting of glycine, sarcosine, proline, serine, alanine, and β-Alanine. In some embodiments, X has a terminal unit, such as a terminal amide unit.

In some embodiments, X is a hydrophilic moiety comprising a sulfate moiety.

In some embodiments, X is represented by Formula (A), (B), or (C):

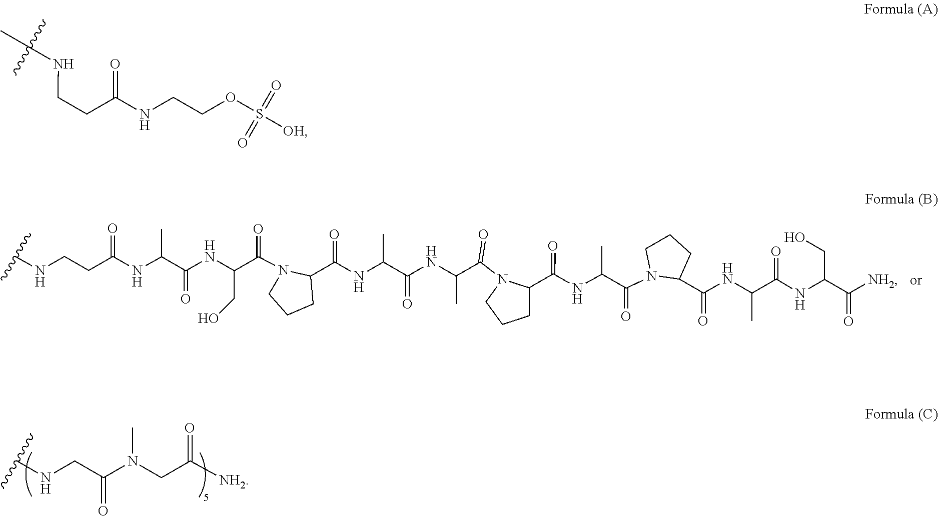

In some embodiments, the ADC comprising the CR1 linker is represented by Formula (II), Formula (IIa), Formula (IIb), Formula (IIe), Formula (IId), Formula (IIe), or Formula (IIf), wherein X is Formula (A), and the linker is denoted as CR1-sulfate.

In some embodiments, the ADC comprising the CR1 linker is represented by Formula (II), Formula (IIa), Formula (IIb), Formula (IIe), Formula (IId), Formula (IIe), or Formula (IIf), wherein X is Formula (B), and the linker is denoted as CR1-PAS10.

In some embodiments, the ADC comprising the CR1 linker is represented by Formula (II), Formula (IIa), Formula (IIb), Formula (IIc), Formula (IId), Formula (IIe), or Formula (IIf), wherein X is Formula (C), and the linker is denoted as CR1-(Gly-Sar)₅-NH₂.

In some embodiments, the drug moiety D comprises exatecan.

As used herein, a linker prior to the chemical reaction to link the Ab (antibody or antigen-binding portion thereof) and D (cytotoxic agent or payload) components of the immunoconjugate is also called a “linker precursor.” It will be apparent to the skilled person in the ADC art whether a certain chemical entity disclosed herein is a linker precursor based on its reactive capabilities, or a linker component in the final immunoconjugate product.

In some embodiments, the linkage between the Ab and D components of the immunoconjugate may be formed through reaction of the components with a homobifunctional linker. Exemplary homobifunctional linkers include, but are not limited to, Lomant's reagent dithiobis (succinimidylpropionate) DSP, 3′3′-dithiobis(sulfosuccinimidyl propionate) (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as, e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), and N,N′-hexamethylene-bis(iodoacetamide).

In some embodiments, the linkage between the Ab and D components of the immunoconjugate may be formed through reaction of the components with a heterobifunctional linker. Exemplary heterobifunctional linkers include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (SMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such asl-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linkage between the Ab and D components of the immunoconjugate may be formed through reaction of the components with a linker having a reactive functional group that may comprise, e.g., a nucleophilic group that is reactive to an electrophilic group present on Ab or D. Exemplary electrophilic groups include carbonyl groups such as aldehydes, ketones, carboxylic acids, esters, amides, enones, acyl halides, and acid anhydrides. Exemplary nucleophilic groups include hydrazide, oxime, amino, hydrazine, thiosemicarbazone, hydrazine carboxylate, and arylhydrazide.

In some embodiments, the conjugation of the linker/payload to the antibody or portion may be formed through reaction with a maleimide group (which may also be referred to as a maleimide spacer). In certain embodiments, the maleimide group is maleimidocaproyl (me); thus, the linker/payload is conjugated to the antibody or portion through reaction between a residue on the antibody or portion and the me group in the linker precursor. In some embodiments, the maleimide group comprises a maleimidomethyl group, such as succinimidyl-4-(N-maleimidomethyl)cyclohexane-1-carboxylate (SMCC) or sulfosuccinimidyl-4-(N-maleimidomethyl)cyclohexane-1-carboxylate (sulfo-SMCC).

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby decreasing the ability of the maleimide to undergo an elimination reaction through a retro-Michael reaction. In some embodiments, the self-stabilizing maleimide is a maleimide group described in Lyon et al., Nat Biotechnol. (2014) 32(10):1059-62. In certain embodiments, the linker precursor comprises a self-stabilizing maleimide. In certain embodiments, the linker precursor is a self-stabilizing maleimide.

Suitable linkers for use in the immunoconjugates of the invention may include, e.g., linkers that are intracellularly cleavable with high extracellular stability. In some embodiments, the linker comprises a functional group that allows for attachment of the linker to any of the antibodies or portions described herein (e.g., a maleimide derivative). In some embodiments, the linker (or precursor) comprises valine-citrulline (VC), C₅ alkyl-VC (C₅-VC), phenylene-C₂ alkyl (Ph-C₂), phenylene-C₃ alkyl (Ph-C₃), C₅ alkyl (C₅), p-aminobenzyloxycarbonyl (PAB), or GGFG (SEQ ID NO: 39). In certain embodiments, the linker (or precursor) comprises a polyethylene glycol (PEG) dimer and valine-alanine (PEG2-VA). In certain embodiments, the linker (or precursor) comprises one of the following: CR1-sulfate, CR1-PAS10, or CR1-(Gly-Sar)s. In particular embodiments, the linker comprises C₅-VC-PAB, Ph-C₂-GGFG, Ph-C₃-GGFG, or C₅-GGFG. In particular embodiments, the linker comprises a CR1-sulfate linker comprising PEG2 and VA (dipeptidyl) moieties, a CR1-PAS10 comprising PEG2 and VA moieties, or a CR1-(Gly-Sar)s comprising PEG2 and VA moieties. In some embodiments, the linker further comprises a spacer between the linker and the cytotoxic moiety. In some embodiments, the spacer is a heteroatom. In some embodiments, the spacer is an alkyl chain. In some embodiments, the spacer is an alkyl chain comprising one or more heteroatoms. In some embodiments, the spacer is a carbonyl group. In some embodiments, the linker is a homobifunctional linker or a heterobifunctional linker.

In some embodiments, the linkers described herein may be attached to the antibodies or antigen-binding portions described herein at a naturally occurring amino acid residue such as a lysine or a free (e.g., reduced) cysteine. In some embodiments, the linkers can be attached to a non-natural amino acid (e.g., azidophenylalanine, p-acetylphenylalanine, or p-azidomethylphenylalanine) by way of an alkyne/azide “click” reaction (for example, dibenzocyclooctyne (DBCO) reagent is one class of click chemistry labeling reagents), carbonyl condensations, Michael-type additions, and Mizoroki-Heck substitutions. Additional linker sites can be added genetically and may comprise a polypeptide motif that allows enzymatic addition of the linker.

In some embodiments, the linker is conjugated to the anti-ROR2 antibody or portion by a chemical ligation process. In some embodiments, the linker is conjugated to the anti-ROR2 antibody or portion by a native ligation. In some embodiments, the conjugation is as described in Dawson et al., Science (1994) 266:776-9; Dawson et al., J Am Chem Soc. (1997) 119:4325-9; Hackeng et al., PNAS (1999) 96:10068-73; or Wu et al., Angew Chem Int Ed. (2006) 45:4116-25. In some embodiments, the conjugation is as described in U.S. Pat. No. 8,936,910. In some embodiments, the linker is conjugated to the anti-ROR2 antibody or portion either site-specifically or non-specifically via native ligation chemistry.

In some embodiments, the linker is conjugated to the anti-ROR2 antibody or antigen-binding portion by a site-directed method utilizing a “traceless” coupling technology (Philochem). In some embodiments, the “traceless” coupling technology utilizes an N-terminal 1,2-aminothiol group on the binding moiety which is then conjugated with the antibody or portion thereof containing an aldehyde group. See, e.g., Casi et al., JACS (2012) 134(13):5887-92. In some embodiments, the linker is conjugated to the anti-ROR2 antibody or portion by a site-directed method utilizing an unnatural amino acid incorporated into the binding moiety. In some embodiments, the unnatural amino acid comprises p-acetylphenylalanine (pAcPhe). In some embodiments, the keto group of pAcPhe is selectively coupled to an alkoxy-amine derivatized conjugating moiety to form an oxime bond. See, e.g., Axup et al., PNAS (2012) 109(40):16101-6.

In some embodiments, the linker is conjugated to the anti-ROR2 antibody or antigen-binding portion by a cysteine conjugated to the anti-ROR2 antibody by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized amino acid molecule via hydrazino-Pictet-Spengler (HIPS) ligation. See, e.g., Wu et al., PNAS (2009) 106(9):3000-5 and Agarwal et al., PNAS (2013) 110(1):46-51.

In some embodiments, the enzyme-catalyzed process comprises microbial transglutaminase (mTG). In certain embodiments, the linker is conjugated to the anti-ROR2 antibody or portion by utilizing a microbial transglutaminase catalyzed process. In some embodiments, mTG catalyzes the formation of a covalent bond between the amide side chain of a glutamine within the recognition sequence and a primary amine of a functionalized amino acid molecule. In some embodiments, mTG is produced from Streptomyces mobarensis. See, e.g., Strop et al., Chemistry and Biology (2013) 20(2):161-7. In some embodiments, the linker is conjugated to the anti-ROR2 antibody by a carbohydrate-based chemical reaction. In the strategy of carbohydrate-based conjugation, the first step is usually to introduce new bioorthogonal functionalities to facilitate conjugation of the antibody to a drug. Strategies that can be used to introduce bioorthogonal functionalities onto the carbohydrate moiety for bioconjugation (glyco-conjugation) include but are not limited to chemical oxidation of glycans; enzymatic and chemo-enzymatic modification of glycans; and metabolic engineering of the carbohydrate moiety. Chemical approaches may use sodium periodate (NaIO4) to oxidize cis-glycol groups of, e.g., galactose or sialic acid to generate aldehydes, which then can be coupled with hydrazide- or primary amine functionalized molecules to create acid-labeled hydrazones or with aminooxy groups to form oximes. Enzymatic and chemo-enzymatic approaches treat the sugar residue with neuraminidase (Neu) and galactose oxidase (Gal Oxi) to form aldehyde functionalities. Continuous treatment of the antibody with β1,4-galactosyltransferase (Gal T)/α2,6-sialyltransferase (Sial T) can yield homogeneously sialylated antibodies. The resulting antibodies then can be selectively oxidized to the corresponding aldehyde functionalities, and if sialic acid derivatives are used, these antibodies can be used as selective bioorthogonal handles. Similarity, the antibody may be treated with β-galactosidase (Gal) and a mutant Gal T to mediate the attachment of bioorthogonal azide- or keto-galactoses to generate homogeneous G2 glycan patterns which possess non-natural functionalities. Another method makes non-canonical thio-fucose derivatives that can be incorporated into the glycan of antibodies by feeding the cells with the bioorthogonal sugar generating expressed antibodies that display thiol functionalities.

Linkers can be conjugated to the anti-ROR2 antibodies and antigen-binding portions of the current disclosure in multiple ways. Generally, a linker and a cytotoxic moiety are synthesized and conjugated before attachment to an antibody. One method of attaching a linker-drug conjugate to an antibody involves reduction of solvent-exposed disulfides with dithiothreitol (DTT) or tris (2-carboxyethyl)phosphine (TCEP), followed by modification of the resulting thiols with maleimide-containing linker-drug moieties. A native antibody contains 4 inter-chain disulfide bonds and 12 intra-chain disulfide bonds, as well as unpaired cysteines. Thus, antibodies modified in this way can comprise greater than one linker-drug moiety per antibody. In certain embodiments, the immunoconjugates described herein comprise at least 1, 2, 3, 4, 5, 6, 7, 8, 9, or 10 linker/drug moieties. In certain embodiments, the immunoconjugates described herein comprise 1 to 10, 1 to 9, 1 to 8, 1 to 7, 1 to 6, 1 to 5, 1 to 4, 1 to 3, 1 to 2, linker/drug moieties, or 1 linker/drug moiety. In cases where the linker is branched and can each attach to multiple drug moieties, the ratio of the drug moiety to the antibody will be higher than using an unbranched linker.

In some embodiments, the immunoconjugate optionally further comprises an endosomolytic moiety. In some cases, the endosomolytic moiety is a cellular compartmental release component, such as a compound capable of releasing from any of the cellular compartments known in the art, such as the endosome, lysosome, endoplasmic reticulum (ER), Golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer. In some embodiments, an endosomolytic polymer described herein is a pH-responsive endosomolytic polymer. A pH-responsive polymer comprises a polymer that increases in size (swells) or collapses depending on the pH of the environment. Polyacrylic acid and chitosan are examples of pH-responsive polymers. In some embodiments, an endosomolytic moiety described herein is a membrane-disruptive polymer. In some cases, the membrane-disruptive polymer comprises a cationic polymer, a neutral or hydrophobic polymer, or an anionic polymer. In some embodiments, the membrane-disruptive polymer is a hydrophilic polymer. In some embodiments, p(alkylacrylic acids) include poly(propylacrylic acid) (polyPAA), poly(methacrylic acid) (PMAA), poly(ethylacrylic acid) (PEAA), and poly(propyl acrylic acid) (PPAA). In some embodiments, a p(alkylacrylic acid) described herein may be a p(alkylacrylic acid) described in Jones et al., Biochemistry Journal (2003) 372:65-75. In some embodiments, a pH-responsive membrane-disruptive polymer comprises p(butyl acrylate-co-methacrylic acid). See, e.g., Bulmus et al., Journal of Controlled Release (2003) 93:105-20; and Yessine et al., Biochimica et Biophysica Acta (2003) 1613:28-38. In some embodiments, a pH-responsive membrane-disruptive polymer comprises p(styrene-alt-maleic anhydride). See, e.g., Henry et al., Biomacromolecules (2006) 7:2407-14. In some embodiments, a pH-responsive membrane-disruptive polymer comprises a pyridyldisulfide acrylate (PDSA) polymer such as poly(MAA-co-PDSA), poly(EAA-co-PDSA), poly(PAA-co-PDSA), poly(MAA-co-BA-co-PDSA), poly(EAA-co-BA-co-PDSA), or poly(PAA-co-BA-co-PDSA) polymer. See, e.g., El-Sayed et al., Journal of Controlled Release (2005) 104:417-27; or Flanary et al., Bioconjugate Chem. (2009) 20:241-8. In some embodiments, the endosomolytic moiety is a lipid (e.g., a fusogenic lipid). In some embodiments, a molecule of Formula A-X-B-Y-C is further conjugated with an endosomolytic lipid (e.g., a fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC). Exemplary small molecules suitable as endosomolytic moieties include, but are not limited to, quinine, chloroquine, hydroxychloroquines, amodiaquins (carnoquines), amopyroquines, primaquines, mefloquines, nivaquines, halofantrines, quinone imines, or any combination thereof.

The term “linkage,” as used herein, refer to a bond or chemical moiety formed from a chemical reaction between the functional group of one molecular entity and another molecule entity. Such bonds may include, but are not limited to, covalent and non-covalent bonds, while such chemical moieties may include, but are not limited to, esters, carbonates, carbamates, imines phosphate esters, hydrazones, acetals, orthoesters, peptide linkages, and oligonucleotide linkages. Hydrolytically stable linkage means that the linkage is substantially stable in water and does not react with water at useful pH values, including but not limited to under physiological conditions, for an extended period of time, perhaps even indefinitely. Hydrolytically unstable or degradable linkage means that the linkage is degradable in water or in aqueous solutions, including, for example, blood. Enzymatically unstable or degradable linkage means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

### E. Exemplary Anti-ROR2 Immunoconjugates

In some embodiments, an anti-ROR2 ADC of the present invention has the formula:

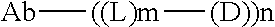

wherein Ab is an anti-ROR2 antibody or an antigen-binding portion thereof (e.g., as described herein), L is a linker (e.g., as described herein) and m is 0 or 1, and D is a cytotoxic drug moiety (e.g., as described herein) and n is an integer from 1 to 10. Due to possible heterogeneity of “n” among molecules in an ADC composition, the average number of drugs conjugated to the antibodies or antigen-binding portions thereof in a composition (drug-to-antibody ratio or DAR) may be a number between consecutive integers. In some embodiments, the DAR is a number between 1 and 10. In some embodiments, the DAR is a number between 3 and 8. In some embodiments, the DAR is a number around 4. In some embodiments, the DAR is 4.

Exemplary immunoconjugates of the present invention are described in Table 2 below, with the DAR numbers obtained through methods of the Working Examples also shown in the Table:

TABLE 2
Summary of Exemplary Anti-ROR2 ADC Constructs

| ID | Ab | Linker | Payload | DAR |
|---|---|---|---|---|
| SLV-A | Ab3 | —C₅—C(═O)-VC-PAB- | MMAE | 4 |
| SLV-B | Ab3 | -Ph(p)-C₃—C(═O)-GGFG-AM- | DXd | 7.1 |
| SLV-C | Ab3 | —C5—C(═O)-VC-PAB- | Duocarmycin | 3.5 |
| | | | derivative | |
| SLV-D | Ab4 | -Ph(p)-C₃—C(═O)-GGFG-AM- | DXd | 7.6 |
| SLV-E | Ab4 | -Ph(m)-C₂—C(═O)-GGFG-AM- | SN-38 | 7.0 |
| SLV-F | Ab4 | —C5—C(═O)-VC-PAB- | Duocarmycin | 3.5 |
| | | | derivative | |
| SLV-G | Ab1 | —C5—C(═O)-VC-PAB- | Duocarmycin | 3.2 |
| | | | derivative | |
| SLV-H | Ab2 | —C5—C(═O)-VC-PAB- | Duocarmycin | 3.2 |
| | | | derivative | |
| SLV-I | Ab1 | CR1(PEG2-VA)-NHCH₂CH₂C(═O)—NHCH₂CH₂—O—S(═O)₂OH | Exatecan | 8.0 |
| SLV-J | Ab1 | CR1(PEG2-VA)-NHCH₂CH₂—C(═O)-PAS10 | Exatecan | 8.0 |
| SLV-K | Ab1 | CR1(PEG2-VA)-(Gly-Sar)₅-NH₂ | Exatecan | 7.9 |
| SLV-L | Ab1 | -Ph(p)-C₃—C(═O)-GGFG-AM- | DXd | 7.5 |
| SLV-M | Ab1 | —C₅—C(═O)-GGFG- | Exatecan | 7.9 |
| SLV-N | Ab1 | —C₅—C(═O)-GGFG-AM- | DXd | 7.8 |
| SLV-O | Ab4 | —C₅—C(═O)-VC-PAB- | MMAF | 4.3 |
| SLV-P | Ab1 | —C₅—C(═O)-VC-PAB- | MMAF | 4.3 |

In the above table, the abbreviations used are as follows: amino methylene (AM); phenylene (Ph); polyethylene glycol (PEG); valine-citrulline (VC); valine-alanine (VA); glycine-glycine-phenylalanine-glycine (GGFG); glycine-sarcosine (Gly-Sar); para-amino-benzyloxycarbonyl (self-immolative moiety) (PAB); proline, alanine, serine-rich sequence (10 residues) (PAS10); monomethyl auristatin E (MMAE); an exatecan derivative (DXd); and core 1 (CR1). “PEG2-VA” indicates the presence of both a PEG2 moiety and a VA moiety within the linker; it should not be understood to mean that the PEG2 moiety is linked directly to the VA moiety.

The linker/payload structures for generating select immunoconjugates are shown in Table 3A. The maleimide group in the linker/payload structure will become a succinimide group once the structure reacts and is conjugated to a cysteine in the antibody or antigen-binding portion. For SLV-K, seven stereochemical isomers are depicted in the tables below.

TABLE 3A
Linker/Payload Structures for Generating Exemplary Anti-ROR2 ADCs

| ADC | Linker/Payload Structure for Generating ADC |
|---|---|
| SLV-A | 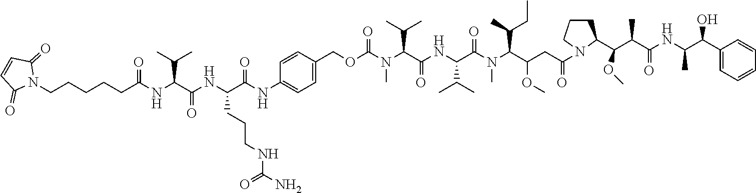 [SMILES: CC[C@H](C)[C@@H](C(CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| | Formula (III) |
| | 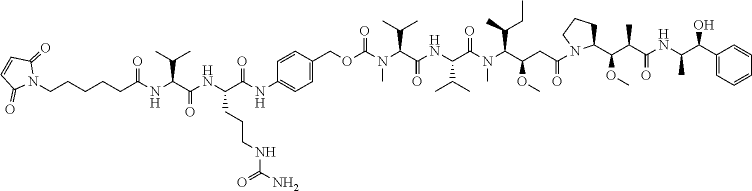 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| | Formula (III.1) |
| SLV-B, SLV-D, SLV-L | 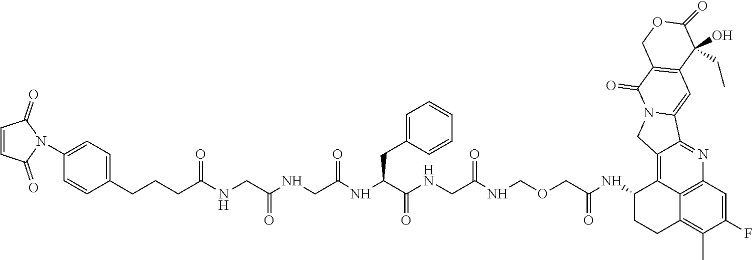 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCc1ccc(N2C(=O)C=CC2=O)cc1)CC3] |
| | Formula (IV) |
| SLV-E | 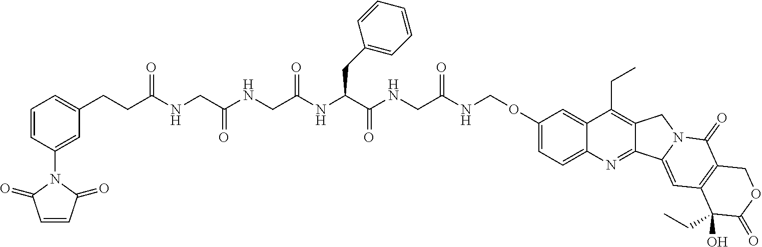 [SMILES: CCc1c2c(nc3ccc(OCNC(=O)CNC(=O)[C@H](Cc4ccccc4)NC(=O)CNC(=O)CNC(=O)CCc4cccc(N5C(=O)C=CC5=O)c4)cc13)-c1cc3c(c(=O)n1C2)COC(=O)[C@]3(O)CC] |
| | Formula (V) |
| SLV-I | 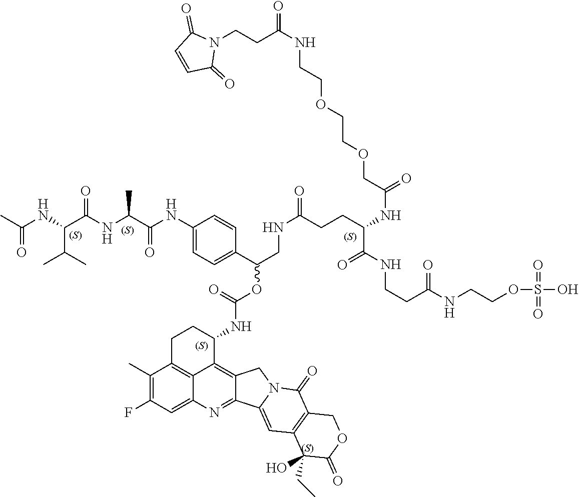 |
| | Formula (VI) |
| | 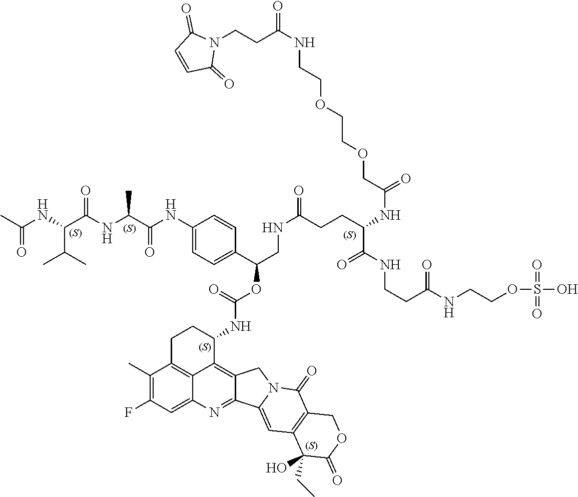 |
| | Formula (VI.1) |
| | 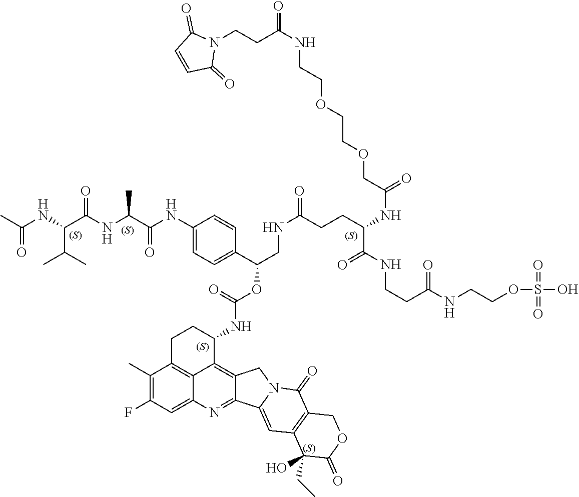 |
| | Formula (VI.2) |
| SLV-J | 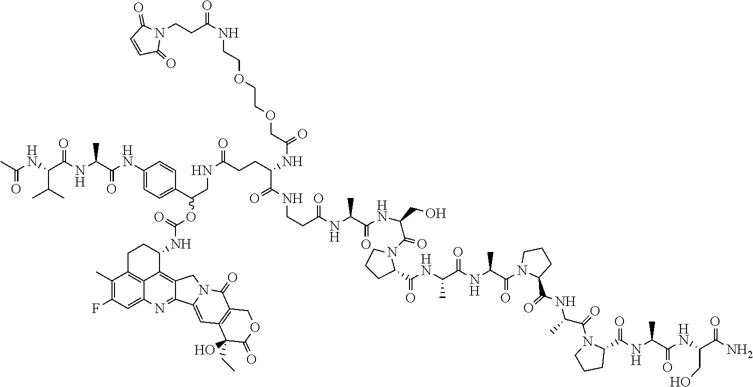 [SMILES: CC[C@@]1(O)C(=O)OCc2c1cc1n(c2=O)Cc2c-1nc1cc(F)c(C)c3c1c2[C@@H](NC(=O)OC(CNC(=O)CC[C@H](NC(=O)COCCOCCNC(=O)CCN1C(=O)C=CC1=O)C(=O)NCCC(=O)N[C@@H](C)C(=O)N[C@@H](CO)C(=O)N1CCC[C@H]1C(=O)N[C@@H](C)C(=O)N[C@@H](C)C(=O)N1CCC[C@H]1C(=O)N[C@@H](C)C(=O)N1CCC[C@H]1C(=O)N[C@@H](C)C(=O)N[C@@H](CO)C(N)=O)c1ccc(NC(=O)[C@H](C)NC(=O)[C@@H](NC(C)=O)C(C)C)cc1)CC3] |
| | Formula (VII) |
| | 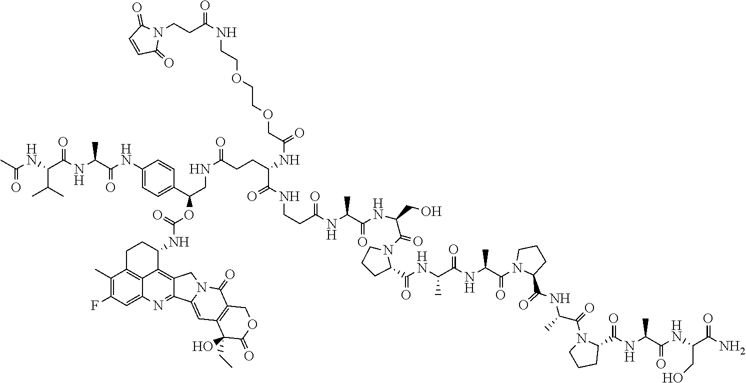 [SMILES: CC[C@@]1(O)C(=O)OCc2c1cc1n(c2=O)Cc2c-1nc1cc(F)c(C)c3c1c2[C@@H](NC(=O)O[C@H](CNC(=O)CC[C@H](NC(=O)COCCOCCNC(=O)CCN1C(=O)C=CC1=O)C(=O)NCCC(=O)N[C@@H](C)C(=O)N[C@@H](CO)C(=O)N1CCC[C@H]1C(=O)N[C@@H](C)C(=O)N[C@@H](C)C(=O)N1CCC[C@H]1C(=O)N[C@@H](C)C(=O)N1CCC[C@H]1C(=O)N[C@@H](C)C(=O)N[C@@H](CO)C(N)=O)c1ccc(NC(=O)[C@H](C)NC(=O)[C@@H](NC(C)=O)C(C)C)cc1)CC3] |
| | Formula (VII.1) |
| | 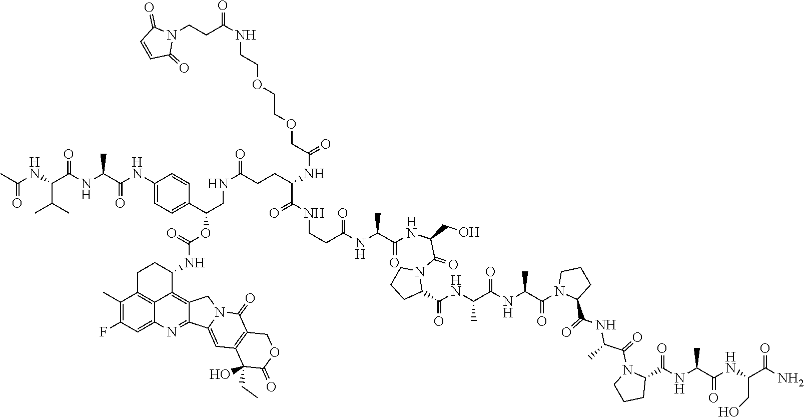 |
| | Formula (VII.2) |
| SLV-K | 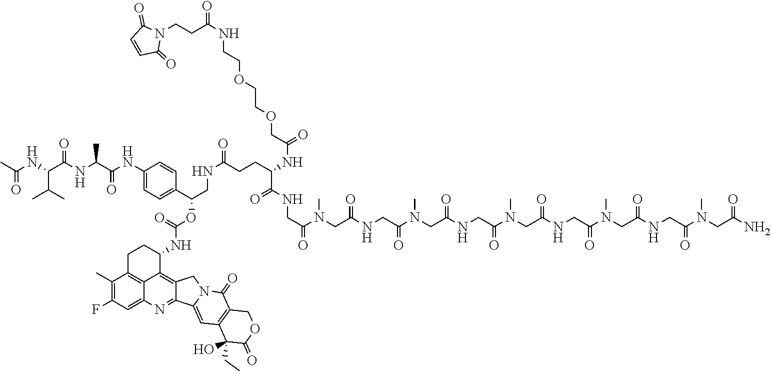 [SMILES: CC[C@@]1(O)C(=O)OCc2c1cc1n(c2=O)Cc2c-1nc1cc(F)c(C)c3c1c2[C@@H](NC(=O)O[C@@H](CNC(=O)CC[C@H](NC(=O)COCCOCCNC(=O)CCN1C(=O)C=CC1=O)C(=O)NCC(=O)N(C)CC(=O)NCC(=O)N(C)CC(=O)NCC(=O)N(C)CC(=O)NCC(=O)N(C)CC(=O)NCC(=O)N(C)CC(N)=O)c1ccc(NC(=O)[C@H](C)NC(=O)[C@@H](NC(C)=O)C(C)C)cc1)CC3] |
| | Formula (VIII.1) |
| | 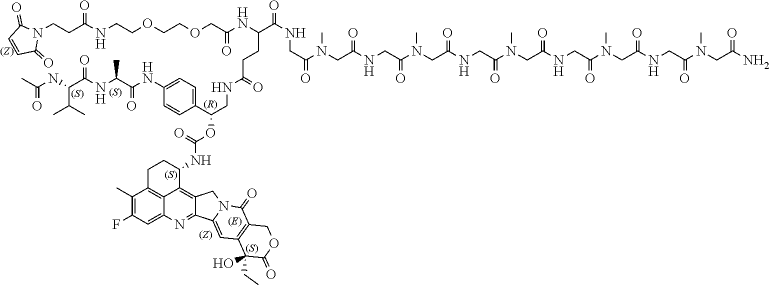 |
| | Formula (VIII.2) |
| | 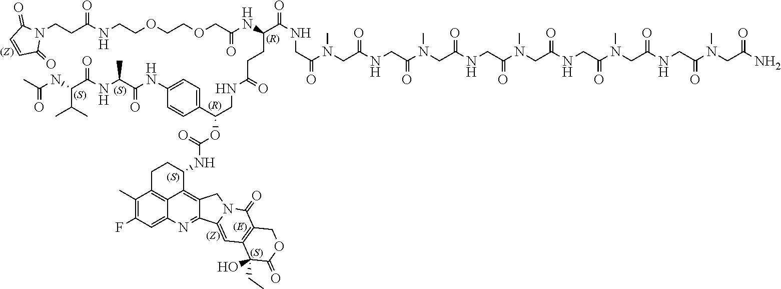 |
| | Formula (VIII.3) |
| | 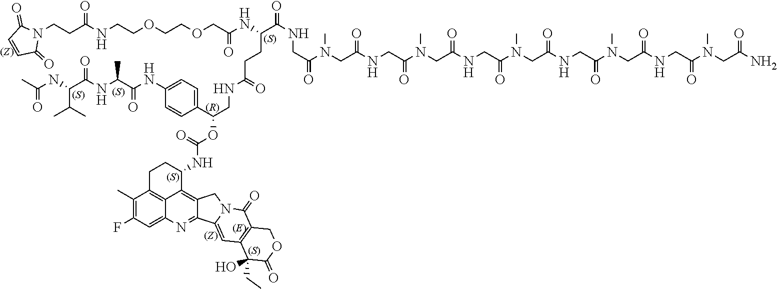 |
| | Formula (VIII.4) |
| | 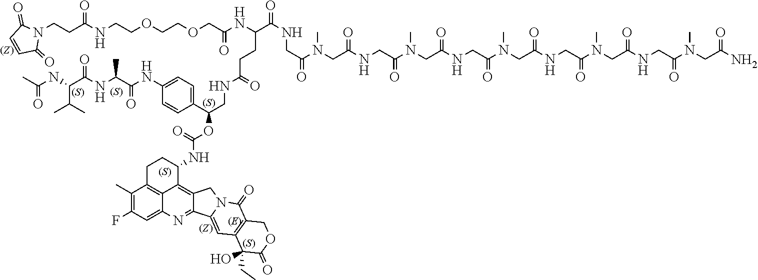 |
| | Formula (VIII.5) |
| | 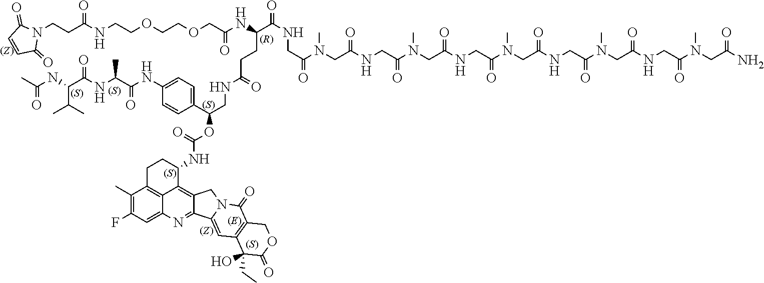 |
| | Formula (VIII.6) |
| | 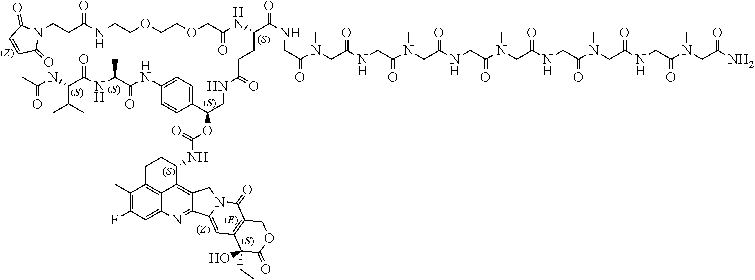 |
| | Formula (VIII.7) |
| SLV-M | 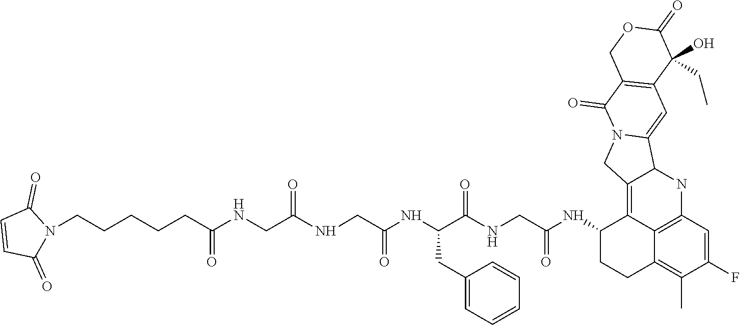 |
| | Formula (IX) |
| SLV-N | 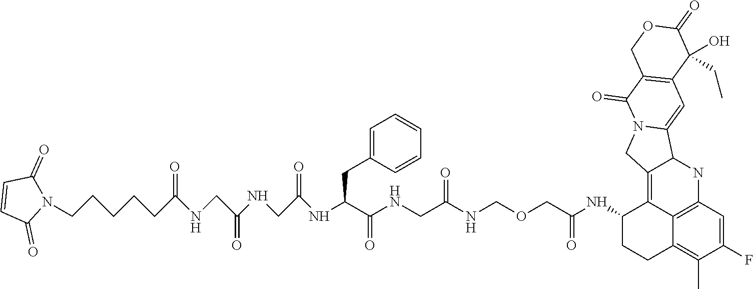 |
| | Formula (X) |
| SLV-O, SLV-P | 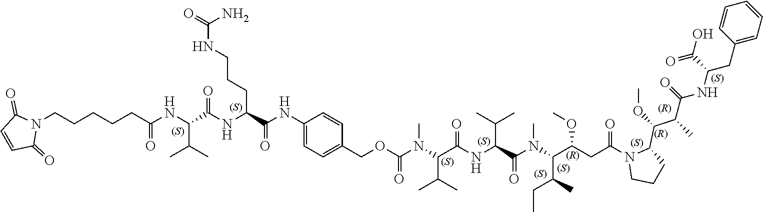 |
| | Formula (XI) |

In some embodiments, the present immunoconjugate is generated by conjugating an anti-ROR2 antibody (e.g., Ab1, Ab2, Ab3, or Ab4) at one or more its cysteine residues (e.g., i to 10 such as 3 to 8) to a linker/payload moiety comprising Formula (III), (III.1), (IV), (V), (VI), (VIII.7), (IX), (X), or (XI), or a pharmaceutically acceptable salt, prodrug, or if appropriate, ester, thereof, through the maleimide conjugation site. Exemplary immunoconjugates are shown in Table 3B, wherein the formula numbers correspond to those in Table 3A but with the added suffix “a,” and wherein for a given immunoconjugate molecule, “n” is an integer between 1 and 10.

TABLE 3B
Chemical Structures of Exemplary Anti-ROR2 ADCs

| ADC | Structure |
|---|---|
| SLV-A | 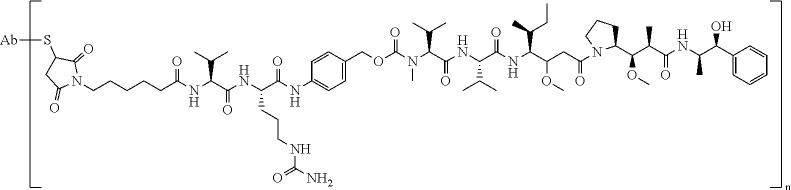 |
| | Formula (IIIa) |
| SLV-B, SLV-D, SLV-L | 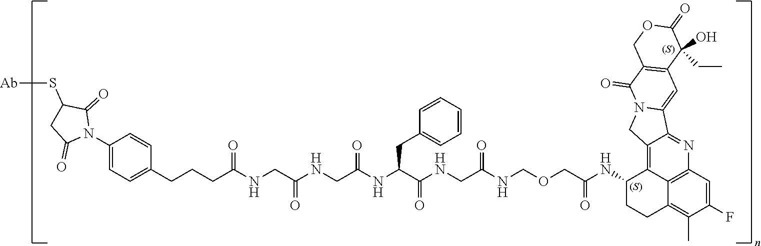 |
| | Formula (IVa) |
| SLV-E | 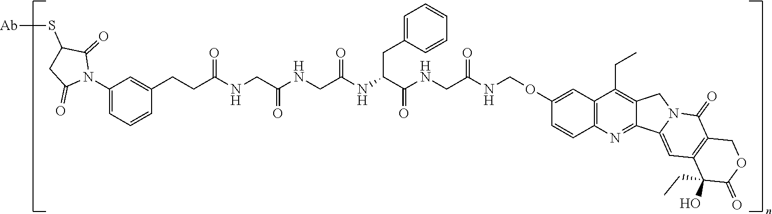 |
| | Formula (Va) |
| SLV-I | 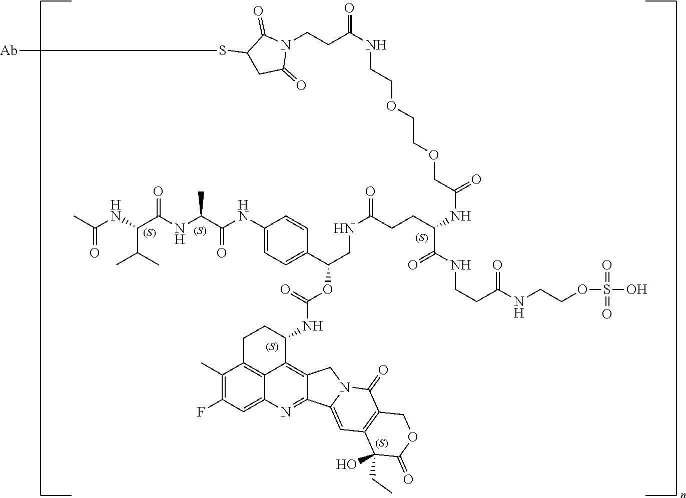 |
| | Formula (VIa) |
| | 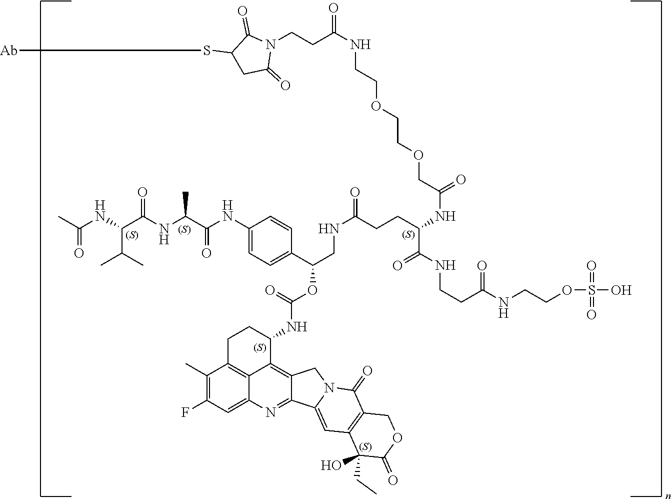 |
| | Formula (VIa.1) |
| | 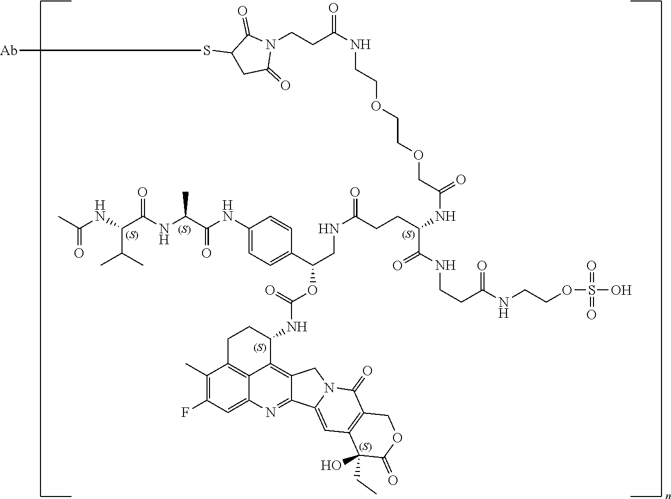 |
| | Formula (VIa.2) |
| SLV-J | 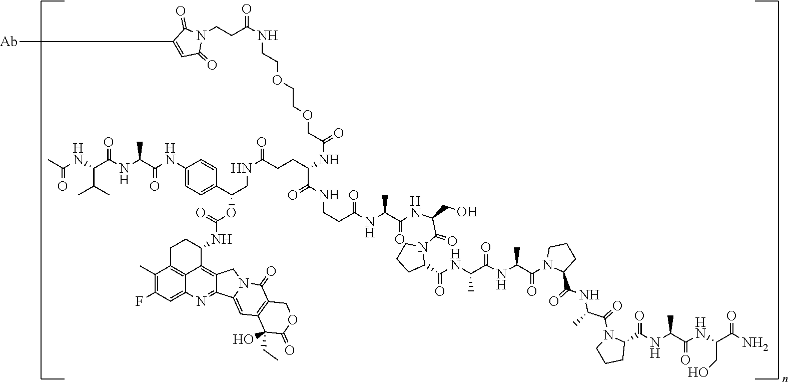 |
| | Formula (VIIa) |
| | 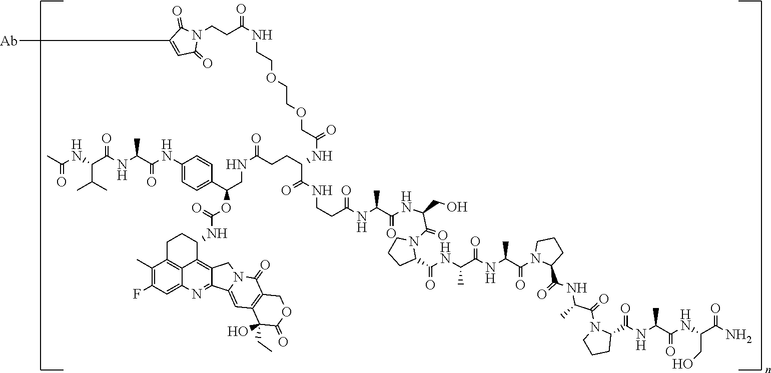 |
| | Formula (VIIa.1) |
| | 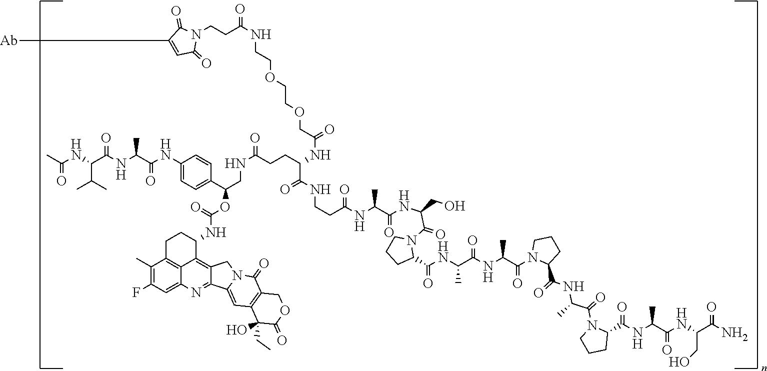 |
| | Formula (VIIa.2) |
| SLV-K | 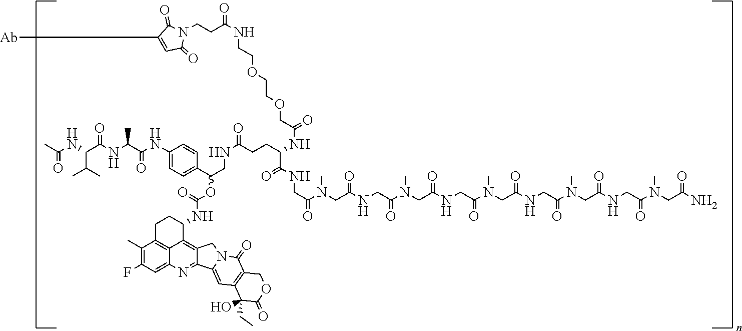 |
| | Formula (VIII.1a) |
| | 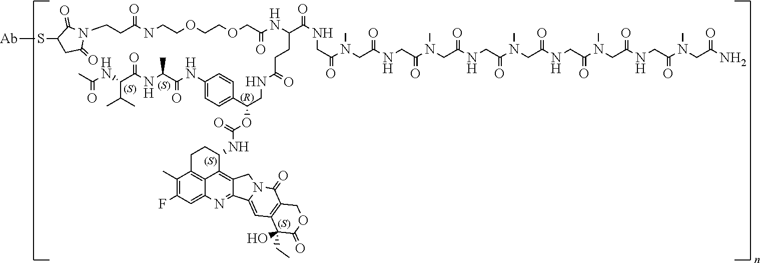 |
| | Formula (VIII.2a) |
| | 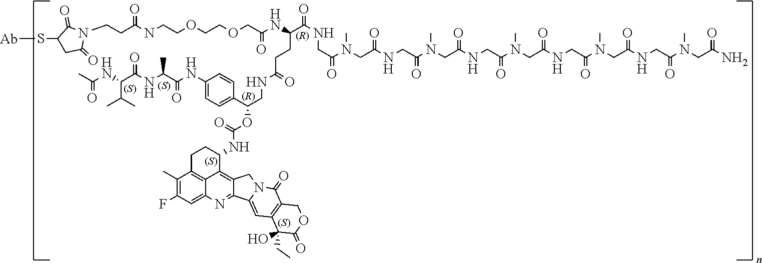 |
| | Formula (VIII.3a) |
| | 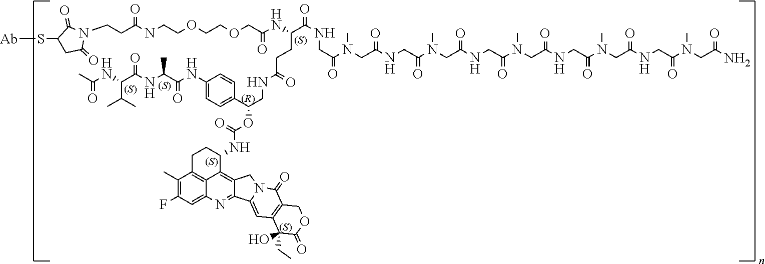 |
| | Formula (VIII.4a) |
| | 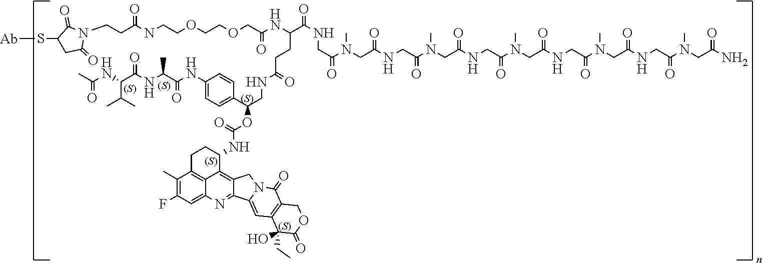 |
| | Formula (VIII.5a) |
| | 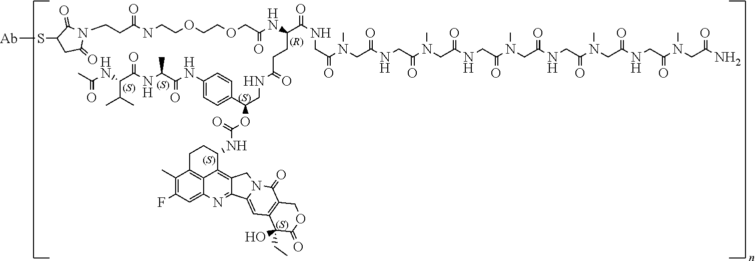 |
| | Formula (VIII.6a) |
| | 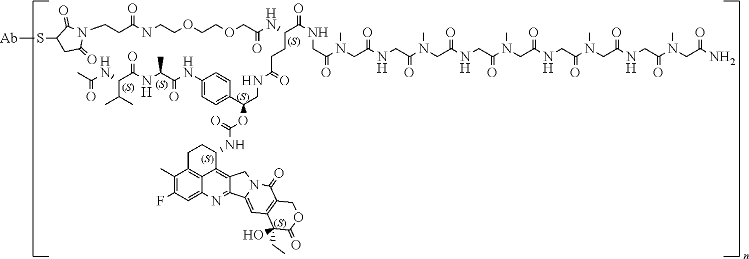 |
| | Formula (VIII.7a) |
| SLV-M | 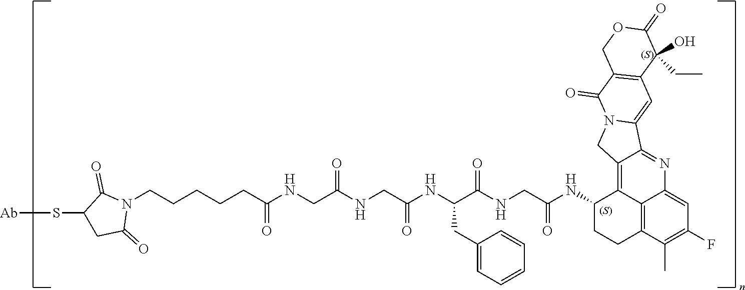 |
| | Formula (IXa) |
| SLV-N | 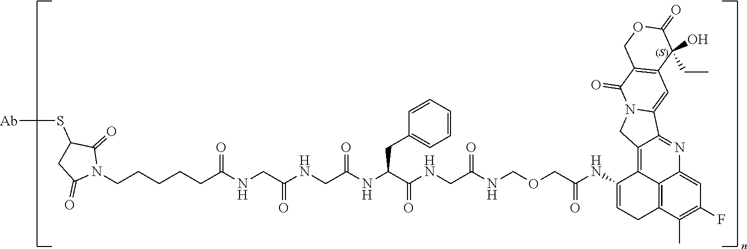 |
| | Formula (Xa) |
| SLV-O, SLV-P | 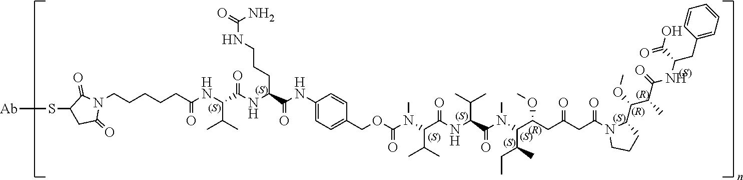 |
| | Formula (XIa) |

In some embodiments, the present immunoconjugate comprises Formula (IIa), (IVa), (Va), (VIa), (VIa.1), (VIa.2), (VIa), (VIIa.1), (VIIa.2), (VIII.1a), (VIII.2a), (VIII.3a), (VIII.4a), (VIII.5a), (VII6a), (VIII.7a), (3Xa), (Xa), or (XIa), where the Ab is Ab and n is 3 to 8 (e.g., 4 or 8). In some embodiments, the immunoconjugate comprises Formula (VIII.1a) and the Ab is Ab1 and n is 3 to 8 (e.g., 4, 7, or 8). In some embodiments, the immunoconjugate comprises Formula (VII2a) and the Ab is Ab1 and n is 3 to 8 (e.g., 4, 7, or 8). In some embodiments, the immunoconjugate comprises Formula (VIII.3a) and the Ab is Ab1 and n is 3 to 8 (e.g., 4, 7, or 8). In some embodiments, the immunoconjugate comprises Formula (VIII.4a) and the Ab is Ab1 and n is 3 to 8 (e.g., 4, 7, or 8). In some embodiments, the immunoconjugate comprises Formula (VIII.5a) and the Ab is Ab1 and n is 3 to 8 (e.g., 4, 7, or 8). In some embodiments, the immunoconjugate comprises Formula (VIII.6a) and the Ab is Ab1 and n is 3 to 8 (e.g., 4, 7, or 8). In some embodiments, the immunoconjugate comprises Formula (VIII.7a) and the Ab is Ab1 and n is 3 to 8 (e.g., 4, 7, or 8). In some embodiments, the immunoconjugate comprises Formula (VIa) and the Ab is Ab1 and n is 3 to 8 (e.g., 4, 7, or 8). In some embodiments, the immunoconjugate comprises Formula (VIa.1) and the Ab is Ab1 and n is 3 to 8 (e.g., 4, 7, or 8). In some embodiments, the immunoconjugate comprises Formula (VIa.2) and the Ab is Ab1 and n is 3 to 8 (e.g., 4, 7, or 8). In some embodiments, the immunoconjugate comprises Formula (VIa) and the Ab is Ab1 and n is 3 to 8 (e.g., 4, 7, or 8). In some embodiments, the immunoconjugate comprises Formula (VIIa.1) and the Ab is Ab1 and n is 3 to 8 (e.g., 4, 7, or 8). In some embodiments, the immunoconjugate comprises Formula (VIIa.2) and the Ab is Ab1 and n is 3 to 8 (e.g., 4, 7, or 8). In some embodiments, the immunoconjugate comprises Formula (IVa) and the Ab is Ab1 and n is 3 to 8 (e.g., 4, 7, or 8). In some embodiments, the immunoconjugate comprises Formula (IXa) and the Ab is Ab1 and n is 3 to 8 (e.g., 4, 7, or 8). In some embodiments, the immunoconjugate comprises Formula (Xa) and the Ab is Ab1 and n is 3 to 8 (e.g., 4, 7, or 8). In some embodiments, the immunoconjugate comprises Formula (XIa) and the Ab is Ab1 and n is 3 to 8 (e.g., 3, 4, or 5).

In some embodiments, the present immunoconjugate comprises Formula (IIIa), (IVa), (Va), (VIa), (VIa.1), (VIa.2), (VIIa), (VIIa.1), (VIIa.2), (VIII.1a), (VIII.2a), (VIII.3a), (VIII.4a), (VIII.5a), (VIII.6a), (VIII.7a), (IXa), (Xa), or (XIa), where the antibody is Ab2 and n is 3 to 8 (e.g., 4 or 8).

In some embodiments, the present immunoconjugate comprises Formula (IIIa), (IVa), (Va), (VIa), (VIa.1), (VIa.2), (VIIa), (VIIa.1), (VIIa.2), (VIII.1a), (VIII.2a), (VIII.3a), (VIII.4a), (VIII.5a), (VIII.6a), (VIII.7a), (IXa), (Xa), or (XIa), where the antibody is Ab3 and n is 3 to 8 (e.g., 4 or 8). In some embodiments, the immunoconjugate comprises Formula (IIIa) and the Ab is Ab3 and n is 3 to 8 (e.g., 3, 4, or 5). In some embodiments, the immunoconjugate comprises Formula (IVa) and the Ab is Ab3 and n is 3 to 8 (e.g., 4, 7, or 8).

In some embodiments, the present immunoconjugate comprises Formula (IIIa), (IVa), (Va), (VIa), (VIa.1), (VIa.2), (VIIa), (VIIa.1), (VIIa.2), (VIII.1a), (VIII.2a), (VIII.3a), (VIII.4a), (VIII.5a), (VIII.6a), (VIII.7a), (IXa), (Xa), or (XIa), where the antibody is Ab4 and n is 3 to 8 (e.g., 4 or 8). In some embodiments, the immunoconjugate comprises Formula (IVa) and the Ab is Ab4 and n is 3 to 8 (e.g., 4, 7, or 8). In some embodiments, the immunoconjugate comprises Formula (Va) and the Ab is Ab4 and n is 3 to 8 (e.g., 4, 7, or 8). In some embodiments, the immunoconjugate comprises Formula (XIa) and the Ab is Ab4 and n is 3 to 8 (e.g., 3, 4, or 5).

### II. Synthesis of Immunoconjugates

The present disclosure also provides methods of synthesizing the ADCs described herein. Exemplary synthesis methods for ADCs listed in Table 2 are described in Example 1 below.

### III. Pharmaceutical Compositions

Another aspect of the present disclosure is a pharmaceutical composition comprising as an active ingredient (or as the sole active ingredient) an anti-ROR2 ADC as described herein. The pharmaceutical composition may additionally comprise a pharmaceutically acceptable excipient. “Pharmaceutically acceptable excipients” may include appropriate solvents, dispersion media, antibacterial and antifungal agents, isotonic agents, and the like. Examples of pharmaceutically acceptable excipients are water and saline (e.g., phosphate-buffered saline).

The pharmaceutical compositions herein may be used to treat cancer, e.g., ROR2-expressing cancer, or ROR2-positive cancer.

A pharmaceutical composition of the present disclosure may comprise a therapeutically effective amount of an ADC described herein. A “therapeutically effective amount” is an amount of the drug (e.g., an ADC described herein) or a pharmaceutical composition comprising it that will relieve to some extent one or more of the symptoms of the disease being treated. A therapeutically effective amount of an anti-cancer therapeutic may, for example, result in delayed tumor growth, elimination of cancer cells, tumor shrinkage, increased survival, slowed or decreased metastasis, or other clinical endpoints desired by healthcare professionals.

In some embodiments, an ADC described herein may be co-administered or formulated with another medication/drug, e.g., for treatment of cancer. The additional medication/drug may comprise, e.g., a chemotherapeutic agent, an anti-neoplastic agent, or an anti-angiogenic agent.

The pharmaceutical compositions described herein may be delivered to a patient through parenteral administration. In particular, parenteral administration is contemplated to include, but is not limited to, subcutaneous, intraperitoneal, intramuscular, intrasternal, intracisternal, intravenous, intraarterial, intrathecal, intraurethral, intracranial, intratumoral, and intrasynovial injection or infusions. In some embodiments, the pharmaceutical composition is delivered intravenously (e.g., through intravenous infusion) or subcutaneously (e.g., through subcutaneous injection).

### IV. Therapeutic Uses

In some embodiments, the anti-ROR2 ADCs of the present disclosure are used to treat cancer in a patient (e.g., a mammal such as a human) in need thereof. In certain embodiments, the cancer is a ROR2-expressing cancer. In certain embodiments, the cancer is a ROR2-positive cancer. The ADC may be administered alone or in combination with other therapeutic agents.

In some embodiments, the cancer treated by the ADCs of the present disclosure may be a solid tumor or a hematopoietic cancer. The cancer may be, e.g., melanoma, skin basal cell cancer, glioblastoma, glioma, gliosarcoma, astrocytoma, meningioma, neuroblastoma, adrenocortical cancer, head and neck cancer (e.g., cancer of the head, neck, nasal cavity, paranasal sinuses, nasopharynx, oral cavity, oropharynx, larynx, hypopharynx, and/or salivary glands, and paragangliomas), oral cancer, salivary gland cancer, nasopharyngeal cancer, breast cancer (e.g., triple negative breast cancer), lung cancer (e.g., non-small cell lung cancer (NSCLC), small cell lung cancer, or squamous cell lung cancer), esophageal cancer, gastroesophageal junction cancer, gastric cancer, gastrointestinal cancer, primary peritoneal cancer, liver cancer, hepatocellular carcinoma, gallbladder cancer, biliary tract cancer, cholangiocarcinoma, colon cancer, rectal cancer, colorectal carcinoma, ovarian cancer, fallopian tube cancer, bladder cancer, upper urinary tract cancer, urothelial cancer, renal cell carcinoma, kidney cancer, genitourinary cancer, cervical cancer, testicular cancer, prostate cancer, fibrosarcoma, liposarcoma, rhabdomyosarcoma (e.g., embryonal rhabdomyosarcoma), leiomyosarcoma, neurofibrosarcoma, synovial sarcoma, liposarcoma, alveolar soft part sarcoma, osteosarcoma, histiocytoma (e.g., malignant fibrous histiocytoma), pancreatic cancer, endometrial cancer, cancer of the appendix, thyroid cancer, advanced Merkel cell cancer, multiple myeloma, sarcomas, choriocarcinoma, leukemia (e.g., erythroleukemia, acute lymphoblastic leukemia, acute monocytic leukemia, acute promyelocytic leukemia, acute myeloid leukemia, acute myelogenous leukemia, chronic myeloid leukemia, chronic myelogenous leukemia, chronic lymphocytic leukemia, acute lymphoblastic leukemia, or mast cell leukemia), lymphoma (e.g., small lymphocytic lymphoma, Burkitt's lymphoma, Hodgkin's lymphoma, non-Hodgkin's lymphoma, diffuse large B cell lymphoma, lymphoplasmacytoid lymphoma, mucosa-associated lymphoid tissue lymphoma, mantle cell lymphoma, T-cell anaplastic large cell lymphoma, follicular lymphoma, monocytic lymphoma, or HTLV-associated T cell leukemia/lymphoma), or mesothelioma. In certain embodiments, the cancer is selected from the group consisting of head and neck cancer, bone cancer (e.g., osteosarcoma), Ewing sarcoma, squamous cell carcinoma, lung cancer (e.g., non-small cell lung cancer or small cell lung cancer), kidney cancer, urethral cancer, colorectal cancer, prostate cancer, glioblastoma multiforme, ovarian cancer, cervical cancer, pancreatic cancer, breast cancer (e.g., triple negative breast cancer), melanoma, liver cancer, bladder cancer, stomach cancer, esophageal cancer, and chronic myelogenous leukemia. In particular embodiments, the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma. The cancer may be, e.g., at an early, intermediate, late, locally advanced, or metastatic stage, and may be relapsed or refractory to other therapeutics, or there may be no standard therapy available.

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. In some embodiments, therapeutic use of an ADC described herein will result in delayed tumor growth, elimination of cancer cells, tumor shrinkage/regression, increased survival, slowed or decreased metastasis, or other clinical endpoints desired by healthcare professionals. In certain embodiments, therapeutic use of an ADC described herein inhibits tumor growth by at least 10, 20, 30, 40, 50, 60, 70, 80, 90, or 100%. In certain embodiments, therapeutic use of an ADC described herein provides partial tumor regression of at least 10, 20, 30, 40, 50, 60, 70, 80, or 90%, or complete tumor regression.

The ADCs of the present disclosure may be administered without additional therapeutic treatments, i.e., as a stand-alone therapy (monotherapy). Alternatively, treatment with the ADCs of the present disclosure may include at least one additional therapeutic treatment (combination therapy), e.g., an immunomodulatory agent, an anti-cancer agent (such as a chemotherapeutic agent, an anti-neoplastic agent, or an anti-angiogenic agent), a vaccine (such as a tumor vaccine), or radiation therapy.

In some embodiments, the additional therapeutic treatment may comprise an anti-cancer agent such as, for example, an agent selected from the group consisting of alkylating agents (e.g., platinum derivatives such as cisplatin, carboplatin and/or oxaliplatin); plant alkaloids (e.g., paclitaxel, docetaxel and/or irinotecan); antitumor antibiotics (e.g., doxorubicin, adriamycin, daunorubicin, epirubicin, idarubicin mitoxantrone, dactinomycin, bleomycin, actinomycin, luteomycin, and/or mitomycin); topoisomerase inhibitors (e.g., topotecan); antimetabolites (e.g., fluorouracil and/or other fluoropyrimidines); kinase inhibitors such as tyrosine kinase inhibitors (e.g., acalabrutinib, ibrutinib, imatinib, sorafenib, lapatinib, etc.); or any combination thereof.

In some embodiments, the additional therapeutic treatment may comprise an agent that modulates immune system activation, including, but not limited to, an agent that modulates the expression or activity of A2AR, A1AR, A2BR, A3AR, ADA, ALP, AXL, BTLA, B7-H3, B7-H4, CD116, CD123, CD27, CD28, CD39, CD40, CD47, CD55, CD73, CD122, CD137, CD160, CGEN-15049, CHK1, CHK2, CTLA-3, CTLA-4, CEACAM (e.g., CEACAM-1 and/or CEACAM-5), EGFR, FLT3, HER2, NKG2A, NKG2AL, GAL9, GITR, HVEM, LAG-3, LILRB1, LY108, LAIR1, MET, NKG2A, ICOS, IDO, IL2R, IL4R, KIR, LAIR1, PAP, PD-1/PD-L1/PD-L2, OX40, STING, TIGIT, TIM-3, TGFR-beta, TLR1, TLR2, TLR3, TLR4, TLR5, TLR6, TLR7, TLR8, TLR9 and TLR10, TNFR2, VEGFR, VEGF, VISTA, LILRB2, CMTM6 and/or 2B4. In certain embodiments, the agent is a small molecule inhibitor, an antisense oligonucleotide, a small interfering RNA, an aptamer, a peptide, or an antibody or an antigen-binding fragment thereof that binds to one of the above molecules.

### V. Articles of Manufacture and Kits

The present disclosure also provides articles of manufacture, e.g., kits, comprising one or more containers (e.g., single-use or multi-use containers) containing a pharmaceutical composition of an anti-ROR2 ADC described herein, optionally an additional therapeutic agent (which may be in the same or a separate pharmaceutical composition), and instructions for use. The ADC, and optional additional therapeutic agent, can be packaged separately in suitable packing such as a vial or ampule made from non-reactive glass or plastic. In certain embodiments, the vial or ampule holds a concentrated stock (e.g., 2x, 5x, 10× or more) of the ADC and optionally the additional therapeutic agent. In certain embodiments, the articles of manufacture such as kits include a medical device for administering the ADC and/or additional therapeutic agent (e.g., a syringe and a needle); and/or an appropriate diluent (e.g., sterile water and normal saline). The present disclosure also includes methods for manufacturing said articles.

### VI. Exemplary Embodiments

Non-limiting, exemplary embodiments of the present disclosure are shown below.

1. An immunoconjugate having the formula of Ab−((L)m−(D))n, wherein:
Ab is an antibody or an antigen-binding portion thereof that specifically binds to human receptor tyrosine kinase like orphan receptor 2 (ROR2);
L is a linker, and m is 0 or 1;
D is a cytotoxic drug moiety; and
n is an integer from 1 to 10.
   2. The immunoconjugate of embodiment 1, wherein the cytotoxic drug moiety is an anti-tubulin agent or a topoisomerase I inhibitor.
   3. The immunoconjugate of embodiment 2, wherein the cytotoxic drug moiety comprises a chemical structure selected from the group consisting of monomethyl auristatin E (MMAE), monomethyl auristatin F (MMAF), exatecan, exatecan derivative (DXd), and SN-38, and pharmaceutically acceptable salts, esters, and analogs thereof.
   4. The immunoconjugate of any one of the preceding embodiments, wherein the linker comprises a cleavable moiety.
   5. The immunoconjugate of any one of the preceding embodiments, wherein the linker comprises
a) one or more of valine-alanine (VA), valine-citrulline (VC), C5-VC, phenylene-C3 (Ph-C3), phenylene-C2 (Ph-C2), C5 alkyl, para-aminobenzyloxycarbonyl (PAB), amino methylene (AM), and GGFG (SEQ ID NO: 39); or
b) one or more of polyethylene glycol (PEG)2-VA, CR1-sulfate, CR1-PAS10, and CR1-(Gly-Sar)5.
   6. The immunoconjugate of embodiment 5, wherein the linker comprises
a) C₅-VC-PAB;
   Ph-C₃-GGFG;
   Ph-C₂-GGFG; or
   C₅-GGFG; or
b) PEG2-VA-CR1-sulfate;
   PEG2-VA-CR1-PAS10; or
   PEG2-VA-CR1-(Gly-Sar)5.
      7. The immunoconjugate of any one of the preceding embodiments, wherein the ratio of the cytotoxic drug moiety to the antibody (DAR) is 3 to 8.
      8. The immunoconjugate of embodiment 1, wherein the immunoconjugate has a structure shown in Table 3A or 3B as
a) SLV-A, SLV-B, SLV-D, SLV-E, SLV-L, SLV-M, SLV-N, SLV-O, or SLV-P; or
b) SLV-I, SLV-J, or SLV-K.
   9. The immunoconjugate of any one of the preceding embodiments, wherein the antibody or antigen-binding portion competes or cross-competes for binding to human ROR2 or binds to the same human ROR2 epitope as an antibody that comprises a heavy chain (HC) and a light chain (LC) comprising
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively.
   10. The immunoconjugate of any one of embodiments 1-9, wherein the antibody or antigen-binding portion comprises heavy chain complementarity-determining region (CDR) 1-3 (HCDR1-3) and light chain CDR1-3 (LCDR1-3) amino acid sequences of
a) SEQ ID NOs: 5, 6, 7, 8, 9, and 10, respectively;
b) SEQ ID NOs: 13, 6, 14, 8, 9, and 10, respectively; or
c) SEQ ID NOs: 19, 6, 20, 8, 9, and 10, respectively.
   11. The immunoconjugate of any one of embodiments 1-9, wherein the antibody or antigen-binding portion comprises
HCDR1 comprising SEQ ID NO: 40;
HCDR2 comprising SEQ ID NO: 28;
HCDR3 comprising SEQ ID NO: 24;
LCDR1 comprising SEQ ID NO: 41;
LCDR2 comprising SEQ ID NO: 9; and
LCDR3 comprising SEQ ID NO: 37.
   12. The immunoconjugate of any one of the preceding embodiments, wherein the antibody or antigen-binding portion comprises heavy chain variable domain (V_{H}) and light chain variable domain (V_{L}) amino acid sequences of
a) SEQ ID NOs: 3 and 4, respectively;
b) SEQ ID NOs: 12 and 4, respectively; or
c) SEQ ID NOs: 17 and 18, respectively.
   13. The immunoconjugate of any one of the preceding embodiments, wherein the antibody is of isotype IgG.
   14. The immunoconjugate of any one of the preceding embodiments, wherein the antibody is of isotype subclass IgG₁, IgG₂, IgG3, or IgG4.
   15. The immunoconjugate of any one of the preceding embodiments, wherein the Fc region of the antibody comprises one or more mutations that reduce effector function.
   16. The immunoconjugate of any one of embodiments 1-12, wherein the antibody comprises HC and LC amino acid sequences of
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively; optionally wherein the HC amino acid sequence lacks the C-terminal lysine.
   17. The immunoconjugate of any one of embodiments 1-12, wherein the antigen-binding portion is an Fab, F(ab)2, or scFv.
   18. A pharmaceutical composition comprising the immunoconjugate of any one of embodiments 1-17 and a pharmaceutically acceptable excipient.
   19. The pharmaceutical composition of embodiment 18, further comprising an additional therapeutic agent selected from the group consisting of an immunomodulatory agent, a chemotherapeutic agent, an anti-neoplastic agent, or an anti-angiogenic agent.
   20. A method of treating cancer in a human patient in need thereof, comprising administering to the patient a therapeutically effective amount of the immunoconjugate of any one of embodiments 1-17.
   21. The method of embodiment 20, wherein the cancer expresses ROR2.
   22. The method of embodiment 20, wherein the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma.
   23. The method of any one of embodiments 20-22, further comprising administering to the patient an additional therapeutic agent.
   24. The method of embodiment 23, wherein the additional therapeutic agent is selected from the group consisting of an immunomodulatory agent, a chemotherapeutic agent, an anti-neoplastic agent, an anti-angiogenic agent, or a tumor vaccine.
   25. The immunoconjugate of any one of embodiments 1-17, or the pharmaceutical composition of embodiment 18 or 19, for use in treating cancer in a method of any one of embodiments 20-24.
   26. Use of the immunoconjugate of any one of embodiments 1-17, or the pharmaceutical composition of embodiment 18 or 19, in the manufacture of a medicament for treating cancer in a method of any one of embodiments 20-24.
   27. A method of making an immunoconjugate, comprising:
providing an antibody or an antigen-binding portion thereof that specifically binds to human receptor tyrosine kinase like orphan receptor 2 (ROR2);
conjugating to the antibody or antigen-binding portion a cytotoxic drug moiety selected from the group consisting of monomethyl auristatin E (MMAE), monomethyl auristatin F (MMAF), exatecan, exatecan derivative (DXd), and SN-38, and pharmaceutically acceptable salts, esters, and analogs thereof; wherein the antibody or antigen-binding portion is as defined in any one of embodiments 9-17.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### Examples

### Example 1: Synthesis of Exemplary Immunoconjugates

### Synthesis of Drug-Linker Compounds

The synthesis schemes of exemplary drug-linker compounds are detailed below.

### A. SLV-I (Compound 9)

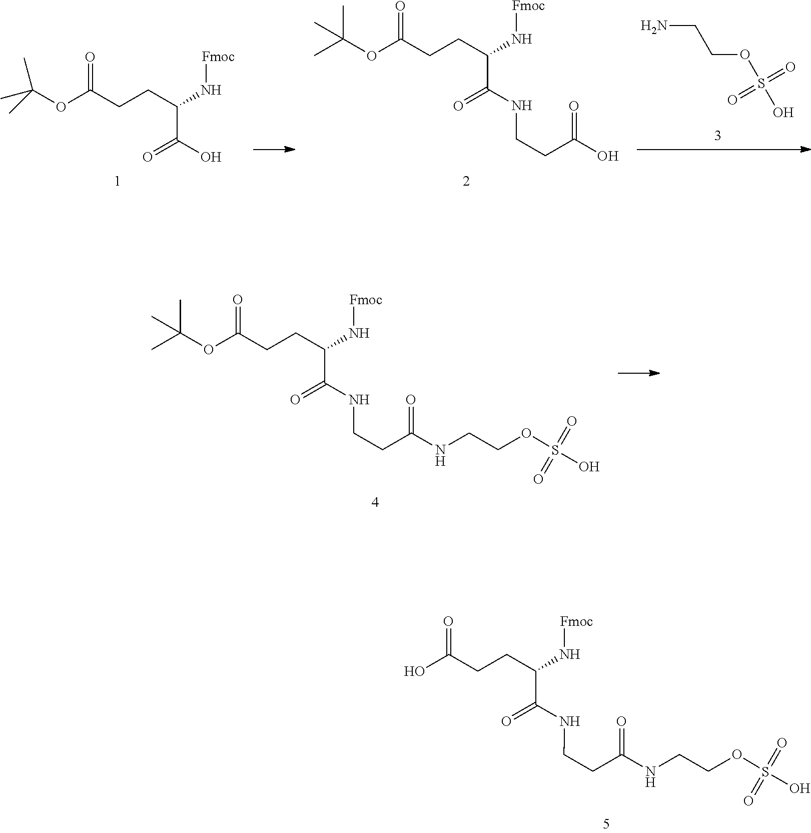

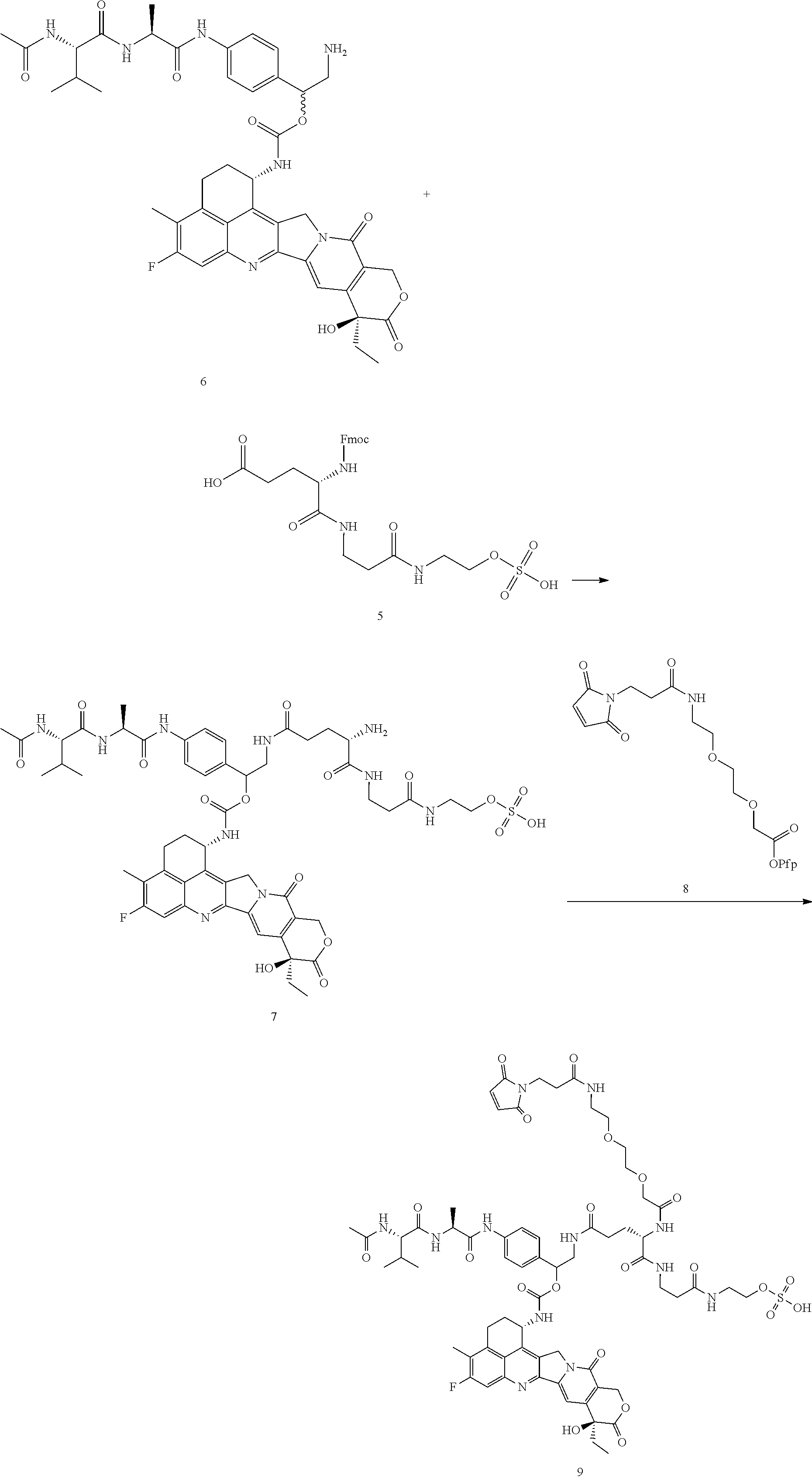

To a solution of Fmoc-Glu(tBu)-COOH·H₂O (1, 44 mg, 0.1 mmol) in DMF (2 mL) was added TSTU (32 mg), followed by DIEA (0.035 mL). The mixture was stirred at room temperature. After 10 min, beta-Alanine (10 mg) in water (0.5 mL) was added, followed by DIEA (0.02 mL). After stirring at room temperature for 30 min, the crude product was purified by RP-HPLC to give compound 2 as a white solid (48 mg), which was dissolved in DMF (2 mL). TSTU (32 mg) was added, followed by DIEA (0.04 mL). After 10 min, the sulfate (3, 28 mg) in water (0.5 mL) was added, followed by DIEA (0.06 mL). After stirring at room temperature for 1 h, the crude product was purified by RP-HPLC to give compound 4 as a white solid (TEA salt, 61 mg).

The compound 4 (61 mg) was treated with TFA/DCM (2/3, v/v, 3 mL) at room temperature for 1 h. The mixture was concentrated to dryness under reduced pressure to give compound 5 as a white solid which was used directly in the next step.

Compound 5 (TEA salt, 20 mg) and compound 6 (TFA salt, 19 mg) were dissolved in anhydrous DMF (2 mL). AOP (12 mg) was added, followed by DIEA (0.02 mL), and the mixture was stirred at room temperature. After 30 mins, piperidine (0.1 mL) was added, and the mixture was stirred at room temperature for 15 min. The crude product was purified by RP-HPLC to give compound 7 as a yellow solid (TFA salt, 18 mg).

Compound 7 (18 mg) and the Pfp ester (8, 9 mg) were dissolved in DMF (2 mL), and DIEA (0.01 mL) was added. After stirring at room temperature for 4 h, the mixture was purified by RP-HPLC to give compound 9 as an off-white solid (16 mg, NH₄⁺ salt) after lyophilization. MS: m/z 1443.4 [M−H]⁻.

### B. SLV-J (Compound 12)

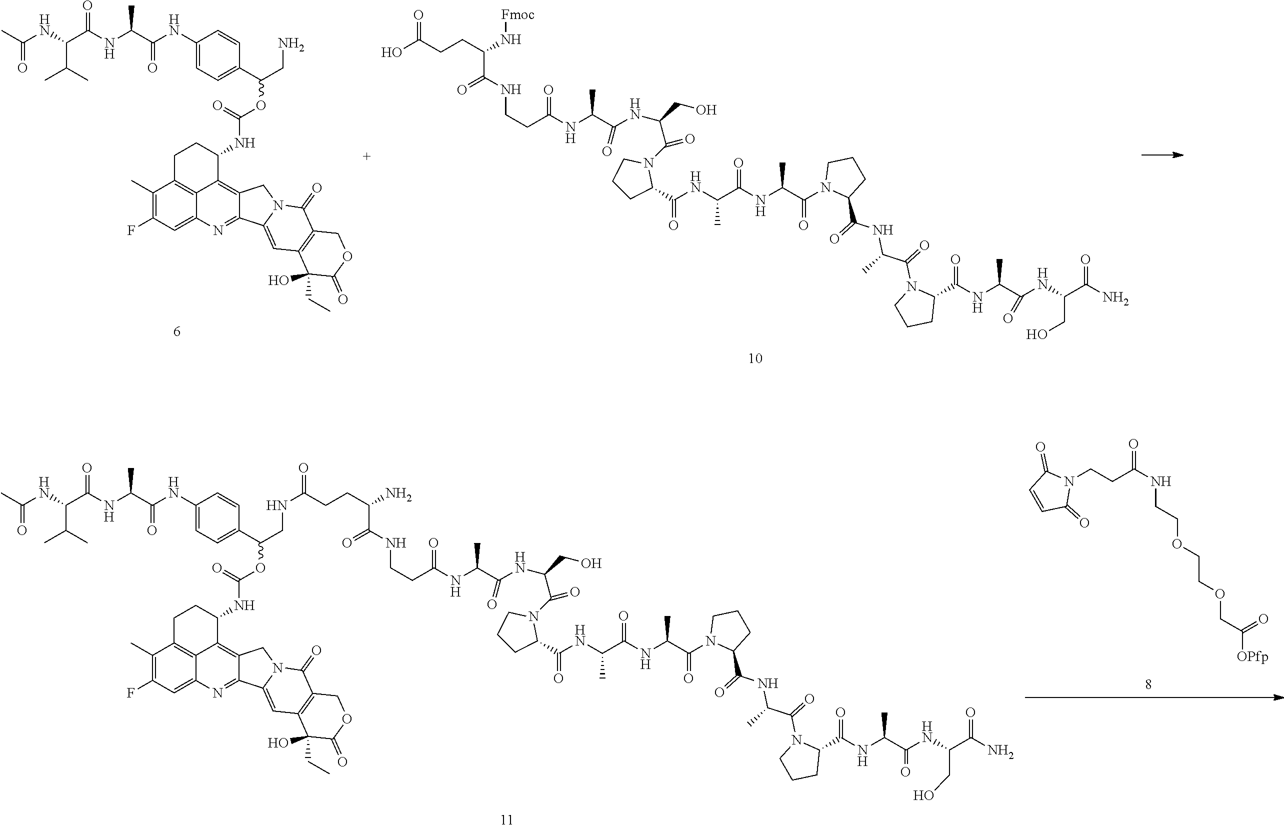

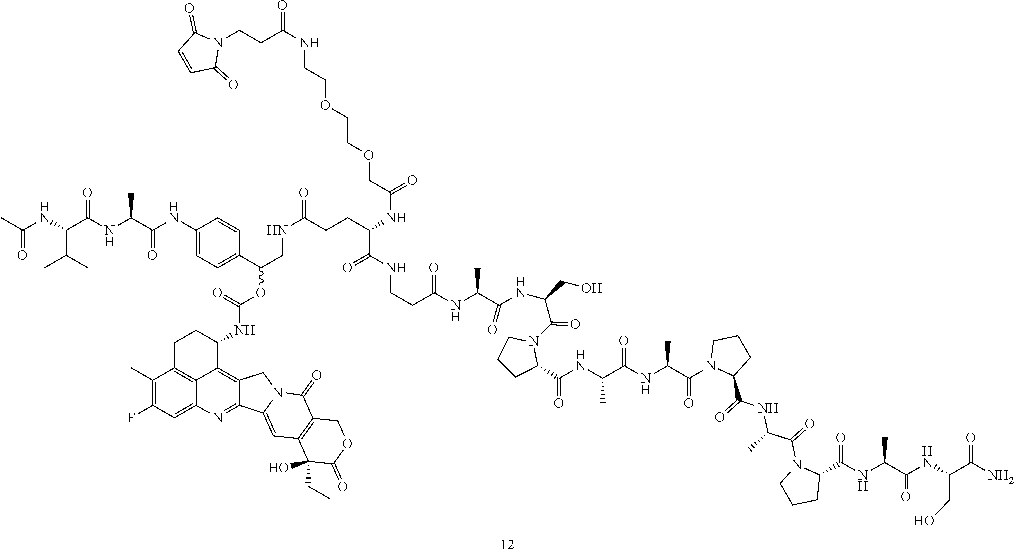 [SMILES: CC[C@@]1(O)C(=O)OCc2c1cc1n(c2=O)Cc2c-1nc1cc(F)c(C)c3c1c2[C@@H](NC(=O)OC(CNC(=O)CC[C@H](NC(=O)COCCOCCNC(=O)CCN1C(=O)C=CC1=O)C(=O)NCCC(=O)N[C@@H](C)C(=O)N[C@@H](CO)C(=O)N1CCC[C@H]1C(=O)N[C@@H](C)C(=O)N[C@@H](C)C(=O)N1CCC[C@H]1C(=O)N[C@@H](C)C(=O)N1CCC[C@H]1C(=O)N[C@@H](C)C(=O)N[C@@H](CO)C(N)=O)c1ccc(NC(=O)[C@H](C)NC(=O)[C@@H](NC(C)=O)C(C)C)cc1)CC3]

Compound 10 (14 mg, purchased from InnoPep, San Diego, CA) and compound 6 (TFA salt, 9 mg) were dissolved in anhydrous DMF (1 mL). PyAOP (6 mg) was added, followed by DIEA (0.007 mL), and the mixture was stirred at room temperature. After 2 hours, piperidine (0.05 mL) was added, and the mixture was stirred at room temperature for 10 min. The crude product was purified by RP-HPLC to give compound 11 as a yellow solid (TFA salt, 17 mg).

Compound 11 (17 mg) and the Pfp ester (8, 5 mg) were dissolved in DMF (1 mL), and DIEA (0.007 mL) was added. After stirring at room temperature for 1 h, the mixture was purified by RP-HPLC to give compound 12 as a yellow solid (17 mg) after lyophilization. MS: m/z 1071.7 [M+2H⁺]/2.

### C. SLV-K (Compound 15)

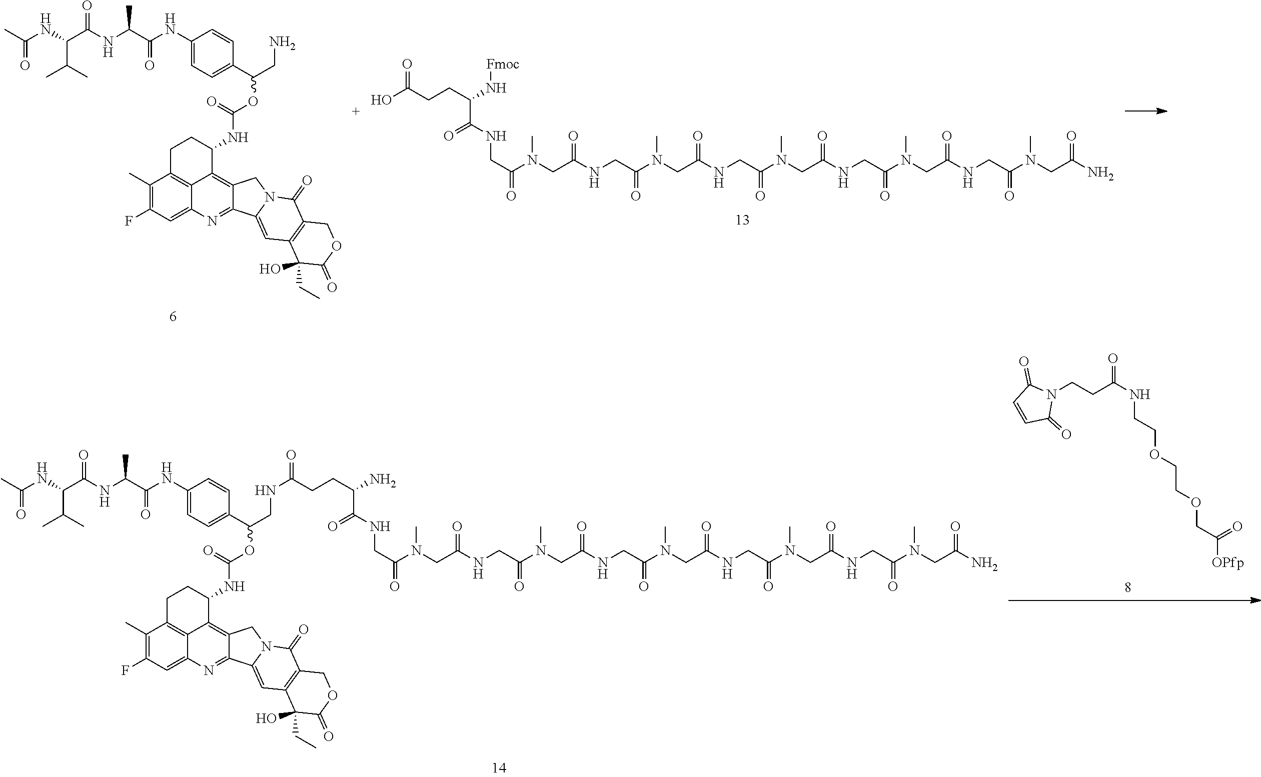

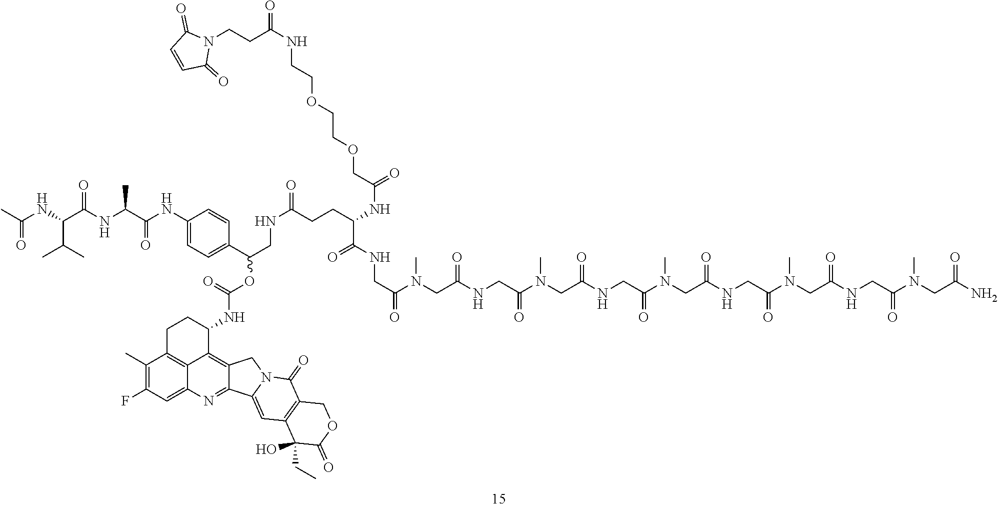 [SMILES: CC[C@@]1(O)C(=O)OCc2c1cc1n(c2=O)Cc2c-1nc1cc(F)c(C)c3c1c2[C@@H](NC(=O)OC(CNC(=O)CC[C@H](NC(=O)COCCOCCNC(=O)CCN1C(=O)C=CC1=O)C(=O)NCC(=O)N(C)CC(=O)NCC(=O)N(C)CC(=O)NCC(=O)N(C)CC(=O)NCC(=O)N(C)CC(=O)NCC(=O)N(C)CC(N)=O)c1ccc(NC(=O)[C@H](C)NC(=O)[C@@H](NC(C)=O)C(C)C)cc1)CC3]

Compound 13 (11 mg, purchased from InnoPep, San Diego, CA) and compound 6 (TFA salt, 9 mg) were dissolved in anhydrous DMF (1 mL). PyAOP (6 mg) was added, followed by DIEA (0.007 mL), and the mixture was stirred at room temperature. After 2 hours, piperidine (0.05 mL) was added, and the mixture was stirred at room temperature for 10 min. The crude product was purified by RP-HPLC to give compound 14 as a yellow solid (TFA salt, 13 mg).

Compound 14 (13 mg) and the Pfp ester (8, 4 mg) were dissolved in DMF (1 mL), and DIEA (0.005 mL) was added. After stirring at room temperature for 1 h, the mixture was purified by RP-HPLC to give compound 15 as a yellow solid (11 mg) after lyophilization. MS: m/z 1891.9 [M+H]⁺.

### D. SLV-K-S(Compound 28)

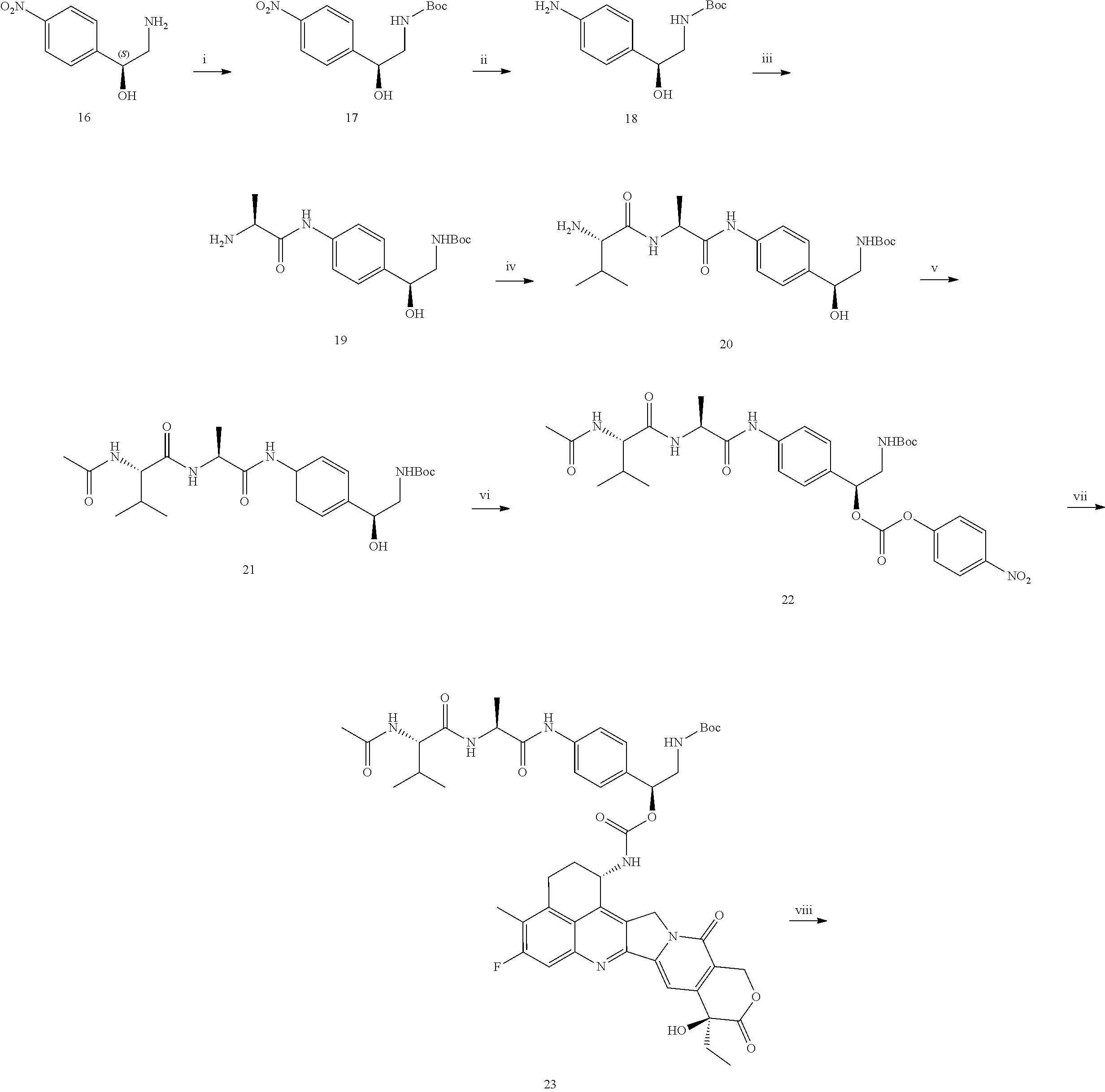

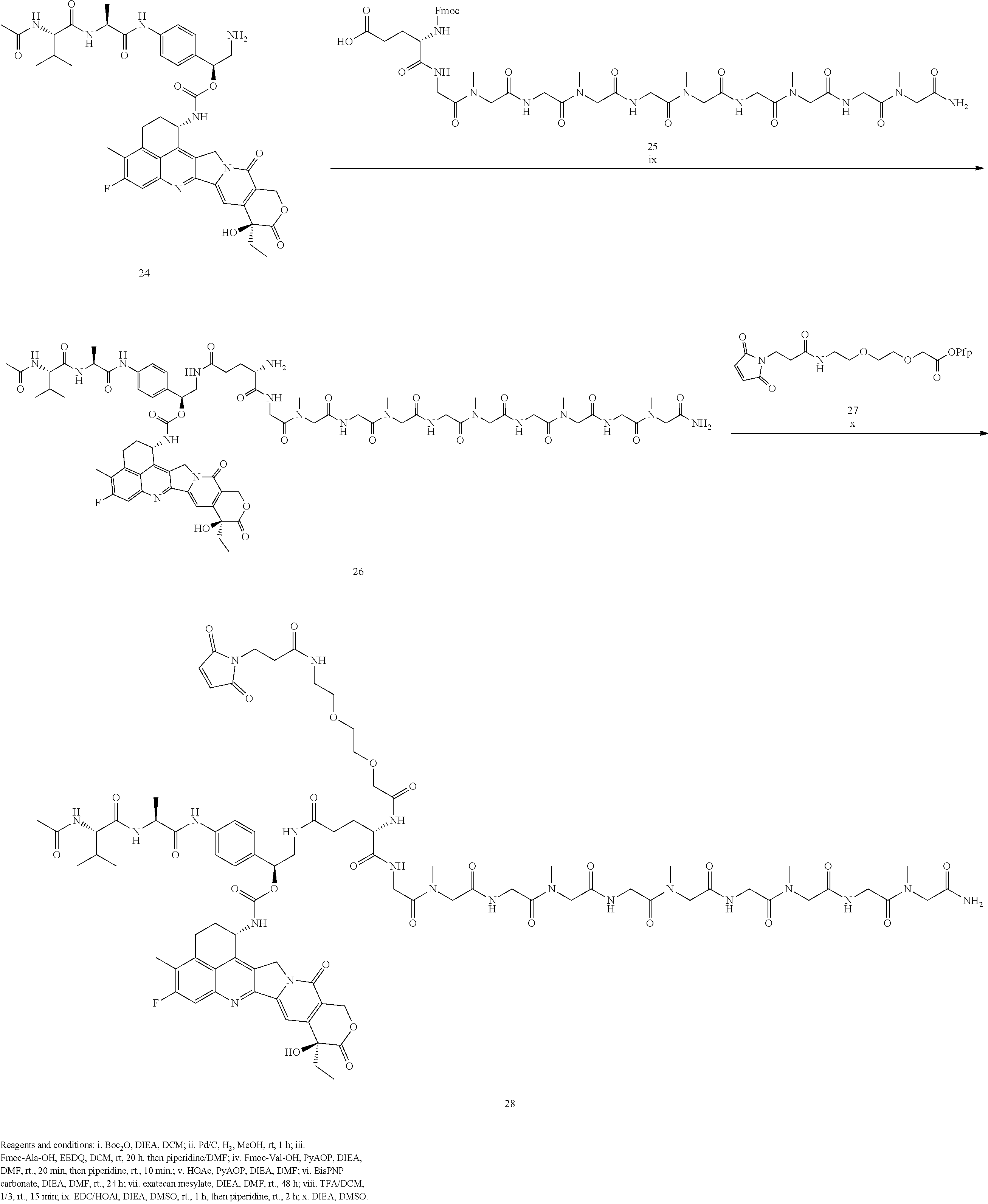

### Step i: tert-butyl (S)-(2-hydroxy-2-(4-nitrophenyl)ethyl)carbamate (Compound 17)

To a solution of (S)-2-amino-1-(4-nitrophenyl)ethan-1-ol (Compound 16) (318 mg, 1.75 mmol) in anhydrous DMF (3 mL) was added DIEA (0.3 mL, 1.75 mmol), and tert-butoxycarbonyl anhydride (381 mg, 1.75 mmol). The mixture was stirred at RT for a period of 1 h. The solvent was evaporated under vacuum and the residue was purified by silica gel column (DCM 99%/MeOH 1%) to give Compound 17 as a tan solid (466 mg, 94%).

### Step ii: tert-butyl (S)-(2-(4-aminophenyl)-2-hydroxyethyl)carbamate (Compound 18)

To a solution of Compound 17 (238 mg, 0.84 mmol) in methanol (6 mL) was added Pd/C (10% Pd, 50 mg). The mixture was purged with argon gas, then filled with hydrogen, and hydrogenated for a period of 1 h. The catalyst was removed by filtration, and the solvent was evaporated to give Compound 18 as a clear solid (197 mg, 92%).

### Step iii: tert-butyl ((S)-2-(4-((S)-2-aminopropanamido)phenyl)-2-hydroxyethyl)carbamate (Compound 19)

To a solution of Compound 18 (197 mg, 0.78 mmol) in DCM (5 mL)/MeOH (1 mL) mixture was added Fmoc-L-Ala-OH (243 mg, 0.78 mmol) and EEDQ (232 mg, 0.94 mmol). The mixture was stirred at RT over a period of 20 h. The solvent was evaporated under vacuum and the resulting residue was dissolved in 2 mL of DMF. Then piperidine (0.3 mL) was added, and the mixture was stirred for 10 min. Then the mixture was purified by RP-HPLC to give Compound 19 as a TFA salt (258 mg, 76%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile Gradient: 2-30% B over 20 min, flow 50 mL/min. The desired compound eluted at 17 min.

### Step iv: tert-butyl ((S)-2-(4-((S)-2-((S)-2-amino-3-methylbutanamido)propanamido)phenyl)-2-hydroxyethyl)carbamate (Compound 20)

To a solution of Compound 19 (TFA salt, 237 mg, 0.54 mmol) in anhydrous DMF (2 mL) was added Fmoc-L-Val-OH (184 mg, 0.54 mmol), PyAOP (283 mg, 0.54 mmol), and DIEA (0.4 mL). The mixture was stirred at RT over a period of 20 min. Then piperidine (0.2 mL) was added, and the mixture was stirred for 10 min. Then the mixture was purified by RP-HPLC to give compound 20 as a TFA salt (230 mg, 79%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 2-35% B over 20 min, flow 50 mL/min. The desired compound eluted at 20 min.

### Step v: tert-butyl ((S)-2-(4-((S)-2-((S)-2-acetamido-3-methylbutanamido)propanamido)phenyl)-2-hydroxyethyl)carbamate (Compound 21)

To a solution of Compound 20 (TFA salt, 230 mg, 0.43 mmol) in anhydrous DMF (2 mL) was added acetic acid (32 mg, 0.53 mol), PyAOP (224 mg, 0.43 mmol) and DIEA (0.3 mL). The mixture was stirred at room temperature for a period of 20 min. Then the mixture was purified directly by RP-HPLC to give Compound 21 as a tan solid (167 mg, 85%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-60% B over 20 min, flow 50 mL/min. The desired compound eluted at 18 min.

### Step vi and Step vii: (S)-1-(4-((S)-2-((S)-2-acetamido-3-methylbutanamido)propanamido) phenyl)-2-((tert-butoxycarbonyl)amino)ethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 23)

To a solution of Compound 21 (104 mg, 0.22 mmol) in anhydrous DMF (2 mL) was added bis(4-nitrophenyl) carbonate (77 mg, 0.25 mol) and DIEA (38 μL). The mixture was stirred at room temperature for a period of 24 h to give a solution of tert-butyl ((S)-2-(4-((S)-2-((S)-2-acetamido-3-methylbutanamido)propanamido)phenyl)-2-(((4-nitrophenoxy)carbonyl)oxy)ethyl)carbamate (compound 22). Then a solution of exatecan mesylate (133 mg, 0.25 mmol) and DIEA (80 μL) in 1 mL of anhydrous DMF was added, and the stirring continued for 2 d. Then the mixture was purified by RP-HPLC to give Compound 23 as a pale-yellow solid (111 mg, 60%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 15-75% B over 20 min, flow 50 mL/min. The desired compound eluted at 20 min.

### Step viii: (S)-1-(4-((S)-2-((S)-2-acetamido-3-methylbutanamido)propanamido)phenyl)-2-aminoethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 24)

Compound 23 (70 mg, 75.7 μmol) was dissolved in a mixture of DCM (3 mL) and TFA (1 mL). The mixture was stirred at room temperature for a period of 15 min, then the solvent was removed under vacuum, and the residue was purified by RP-HPLC to give Compound 24 as a TFA salt (58 mg, 81%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-50% B over 20 min, flow 50 mL/min. The desired compound eluted at 17 min.

### Step ix: (32S,38S)-38-(4-((S)-2-((S)-2-acetamido-3-methylbutanamido)propanamido)phenyl)-1,32-diamino-3,9,15,21,27-pentamethyl-1,4,7,10,13,16,19,22,25,28,31,35-dodecaoxo-3,6,9,12,15,18,21,24,27,30,36-undecaazaoctatriacontan-38-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 26)

To a solution of Compound 24 (TFA salt, 30 mg, 32 μmol) in anhydrous DMF (2 mL) was added (S)-32-((((9H-fluoren-9-yl)methoxy)carbonyl)amino)-1-amino-3,9,15,21,27-pentamethyl-1,4,7,10,13,16,19,22,25,28,31-undecaoxo-3,6,9,12,15,18,21,24,27,30-decaazapentatriacontan-35-oic acid (Compound 25) (32 mg, 32 μmol), PyAOP (17 mg, 32 μmol) and DIEA (23 μL). The mixture was stirred at room temperature for a period of 90 min. Then piperidine (150 μL) was added and the stirring continued for an additional 10 min. Then the mixture was purified directly by RP-HPLC to give Compound 26 as a TFA salt (34 mg, 62%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 2-45% B over 20 min, flow 50 mL/min. The desired compound eluted at 17 min.

### Step x: (32S,38S)-38-(4-((S)-2-((S)-2-acetamido-3-methylbutanamido)propanamido)phenyl)-1-amino-32-(2-(2-(2-β-(2,5-dioxo-2,5-dihydro-1H-pyrrol-1-yl)propanamido)ethoxy) ethoxy)acetamido)-3,9,15,21,27-pentamethyl-1,4,7,10,13,16,19,22,25,28,31,35-dodecaoxo-3,6,9,12,15,18,21,24,27,30,36-undecaazaoctatriacontan-38-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 28)

To a solution of Compound 26 (TFA salt, 34 mg, 20 μmol) in anhydrous DMF (1 mL) was added perfluorophenyl 2-(2-(2-β-(2,5-dioxo-2,5-dihydro-1H-pyrrol-1-yl)propanamido) ethoxy)ethoxy)acetate (Compound 27) (10 mg, 20 μmol) and DIEA (14 μL). The mixture was stirred at room temperature for a period of 30 min. Then the mixture was purified directly by RP-HPLC to give Compound 28 as a pale-yellow solid (26 mg, 69%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-50% B over 20 min, flow 50 mL/min. The desired compound eluted at 17 min. MS [M+2H⁺]/2=946.2.

### E. SLV-K-R (Compound 39)

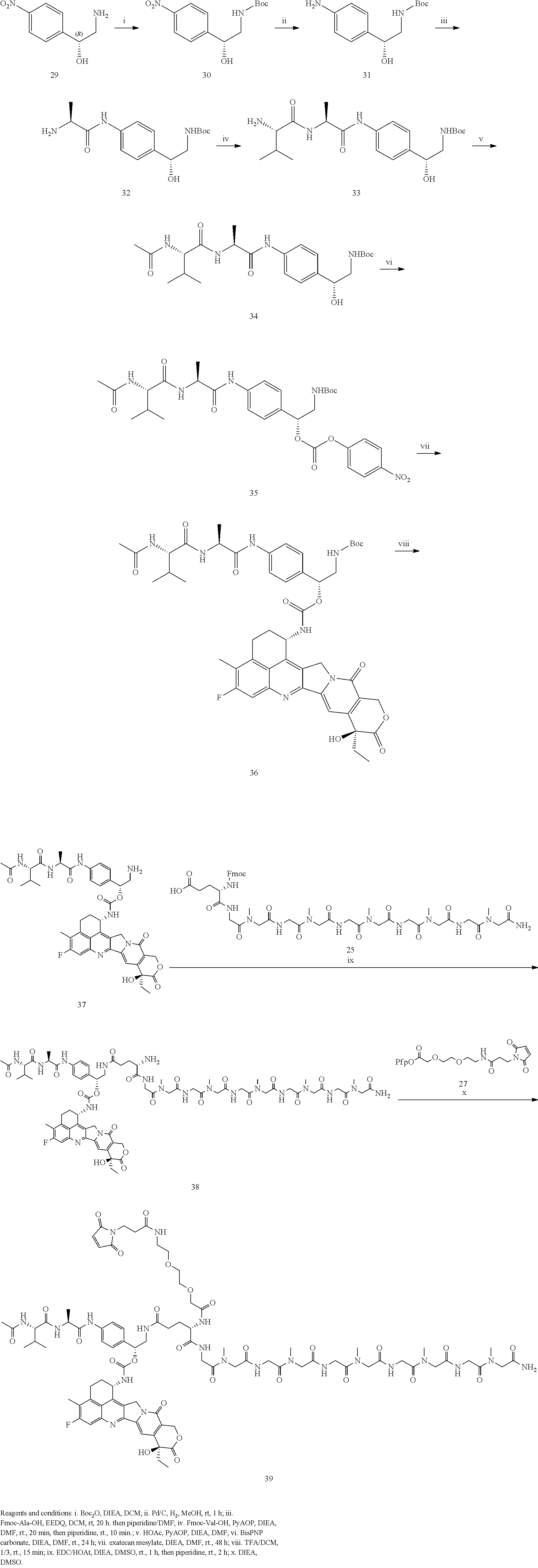

Step i: tert-butyl (R)-(2-hydroxy-2-(4-nitrophenyl)ethyl)carbamate (Compound 30)

To a solution of (R)-2-amino-1-(4-nitrophenyl)ethan-1-ol (Compound 29) (318 mg, 1.75 mmol) in anhydrous DMF (3 mL) was added DIEA (0.3 mL, 1.75 mmol), and tert-butoxycarbonyl anhydride (381 mg, 1.75 mmol). The mixture was stirred at RT for a period of 1 h. The solvent was evaporated under vacuum and the residue was purified by silicagel column (DCM 99%/MeOH 1%) to give Compound 30 as a tan solid (466 mg, 94%).

### Step ii: tert-butyl (R)-(2-(4-aminophenyl)-2-hydroxyethyl)carbamate (Compound 31)

To a solution of Compound 30 (238 mg, 0.84 mmol) in methanol (6 mL) was added Pd/C (10% Pd, 50 mg). The mixture was purged with argon gas, then filled with hydrogen, and hydrogenated for a period of 1 h. The catalyst was removed by filtration, and the solvent was evaporated to give Compound 31 as a clear solid (197 mg, 92%).

### Step iii: tert-butyl ((R)-2-(4-((S)-2-aminopropanamido)phenyl)-2-hydroxyethyl)carbamate (Compound 32)

To a solution of Compound 31 (197 mg, 0.78 mmol) in DCM (5 mL)/MeOH (1 mL) mixture was added Fmoc-L-Ala-OH (243 mg, 0.78 mmol) and EEDQ (232 mg, 0.94 mmol). The mixture was stirred at RT over a period of 20 h. The solvent was evaporated under vacuum and the resulting residue was dissolved in 2 mL of DMF. Then piperidine (0.3 mL) was added, and the mixture was stirred for 10 min. Then the mixture was purified by RP-HPLC to give Compound 32 as a TFA salt (258 mg, 76%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 2-30% B over 20 min, flow 50 mL/min. The desired compound eluted at 17 min.

### Step iv: tert-butyl ((R)-2-(4-((S)-2-((S)-2-amino-3-methylbutanamido)propanamido)phenyl)-2-hydroxyethyl)carbamate (Compound 33)

To a solution of Compound 32 (TFA salt, 237 mg, 0.54 mmol) in anhydrous DMF (2 mL) was added Fmoc-L-Val-OH (184 mg, 0.54 mmol), PyAOP (283 mg, 0.54 mmol), and DIEA (0.4 mL). The mixture was stirred at RT over a period of 20 min. Then piperidine (0.2 mL) was added, and the mixture was stirred for 10 min. Then the mixture was purified by RP-HPLC to give Compound 112 as a TFA salt (230 mg, 79%). Preparative HPLC method details: Column: Phenomenex Gemini NX 5, C18, 110 Å, 150×50 mm. Instrument: Shimadzu LC with CTC IFC Mobile phase: A) water (0.1% TFA), B) acetonitrile. Gradient: 2-35% B over 20 min, flow 50 mL/min. The desired compound eluted at 20 min.

### Step v: tert-butyl ((R)-2-(4-((S)-2-((S)-2-acetamido-3-methylbutanamido)propanamido) phenyl)-2-hydroxyethyl)carbamate (Compound 34)

To a solution of Compound 33 (TFA salt, 230 mg, 0.43 mmol) in anhydrous DMF (2 mL) was added acetic acid (32 mg, 0.53 mol), PyAOP (224 mg, 0.43 mmol) and DIEA (0.3 mL). The mixture was stirred at room temperature for a period of 20 min. Then the mixture was purified directly by RP-HPLC to give Compound 34 as a tan solid (167 mg, 85%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-60% B over 20 min, flow 50 mL/min. The desired compound eluted at 18 min.

### Step vi and Step vii: (R)-1-(4-((S)-2-((S)-2-acetamido-3-methylbutanamido)propanamido) phenyl)-2-((tert-butoxycarbonyl)amino)ethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 36)

To a solution of Compound 34 (104 mg, 0.22 mmol) in anhydrous DMF (2 mL) was added bis(4-nitrophenyl) carbonate (77 mg, 0.25 mol) and DIEA (38 μL). The mixture was stirred at room temperature for a period of 24 h to give a solution of tert-butyl ((R)-2-(4-((S)-2-((S)-2-acetamido-3-methylbutanamido)propanamido)phenyl)-2-(((4-nitrophenoxy)carbonyl)oxy)ethyl) carbamate (Compound 35). Then a solution of exatecan mesylate (133 mg, 0.25 mmol) and DIEA (80 μL) in 1 mL of anhydrous DMF was added, and the stirring continued for 2 d. Then the mixture was purified by RP-HPLC to give Compound 115 as a pale-yellow solid (111 mg, 60%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 15-75% B over 20 min, flow 50 mL/min. The desired compound eluted at 20 min.

### Step viii: (R)-1-(4-((S)-2-((S)-2-acetamido-3-methylbutanamido)propanamido)phenyl)-2-aminoethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 37)

Compound 36 (70 mg, 75.7 μmol) was dissolved in a mixture of DCM (3 mL) and TFA (1 mL). The mixture was stirred at room temperature for a period of 15 min, then the solvent was removed under vacuum, and the residue was purified by RP-HPLC to give Compound 37 as a TFA salt (58 mg, 81%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-50% B over 20 min, flow 50 mL/min. The desired compound eluted at 17 min.

### Step ix: (32S,38R)-38-(4-((S)-2-((S)-2-acetamido-3-methylbutanamido)propanamido)phenyl)-1,32-diamino-3,9,15,21,27-pentamethyl-1,4,7,10,13,16,19,22,25,28,31,35-dodecaoxo-3,6,9,12,15,18,21,24,27,30,36-undecaazaoctatriacontan-38-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 38)

To a solution of Compound 37 (TFA salt, 30 mg, 32 μmol) in anhydrous DMF (2 mL) was added Compound 25 (32 mg, 32 μmol), PyAOP (17 mg, 32 μmol) and DIEA (23 μL). The mixture was stirred at room temperature for a period of 90 min. Then piperidine (150 μL) was added and the stirring continued for an additional 10 min. Then the mixture was purified directly by RP-HPLC to give Compound 38 as a TFA salt (34 mg, 62%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 2-45% B over 20 min, flow 50 mL/min. The desired compound eluted at 17 min.

### Step x: (32S,38R)-38-(4-((S)-2-((S)-2-acetamido-3-methylbutanamido)propanamido)phenyl)-1-amino-32-(2-(2-(2-β-(2,5-dioxo-2,5-dihydro-1H-pyrrol-1-yl)propanamido)ethoxy) ethoxy)acetamido)-3,9,15,21,27-pentamethyl-1,4,7,10,13,16,19,22,25,28,31,35-dodecaoxo-3,6,9,12,15,18,21,24,27,30,36-undecaazaoctatriacontan-38-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (Compound 39)

To a solution of Compound 38 (TFA salt, 34 mg, 20 μmol) in anhydrous DMF (1 mL) was added Compound 27 (10 mg, 20 μmol) and DIEA (14 μL). The mixture was stirred at room temperature for a period of 30 min. Then the mixture was purified directly by RP-HPLC to give Compound 39 as a pale-yellow solid (26 mg, 69%). Preparative HPLC method details: Column: Phenomenex Gemini NX 5, C18, 110 Å, 150×50 mm. Instrument: Shimadzu LC with CTC IFC. Mobile phase: A) water (0.1% TFA), B) acetonitrile. Gradient: 5-50% B over 20 min, flow 50 mL/min. The desired compound eluted at 17 min. MS [M+2H⁺]/2=946.3

### Production of Antibody ADCs

Multiple ROR2 antibody ADCs were synthesized and characterized. Four different versions of the murine ROR2 antibody 6E6 were used: Ab3 (chimeric 6E6), Ab4 (effectorless chimeric 6E6), Ab2 (humanized, effectorless 6E6, version 2), and Ab1 (humanized, effectorless 6E6, version 1). Additionally, six different payloads (MMAE, MMAF, SN-38, exatecan, DXd, and a duocarmycin derivative), and nine different linkers were used. All of the linkers were cleavable and all conjugations were performed using maleimide chemistry to generate ADCs with a DAR of 3-4 or 7.9-8. The ADCs synthesized are summarized in Table 2 above.

The following general protocol was used for conjugations. First, the pH of 50 mg of antibody in various formulations was adjusted to a final pH of 7-8 using 0.5 M Tris, 0.025 M EDTA, pH 8.5. The addition of 0.025 M EDTA in the formulation buffer was added to prevent metal-catalyzed disulfide reoxidation. To achieve a DAR˜8, 7 equivalents (eq) of tris(2-carboxyethyl)phosphine (TCEP, 10 mM) was added to the antibody and incubated for 90 min at 25° C. Next, 12 eq of 20 mM maleimide (MC) containing linker payload in 10% N,N-dimethylacetamide (DMA) was added for 60 min at 25° C. To achieve a DAR 3-4, 2-2.5 eq of tris(2-carboxyethyl)phosphine (TCEP, 10 mM) was added to the antibody and incubated for 90 min at 25° C. Next, 8 eq of 20 mM linker payload in 10% N,N-dimethylacetamide (DMA) was added for 60 min at 25° C. Extra solvent was added to the reduced antibody prior to linker payload addition (final solvent concentration following the addition of toxin was 10% v/v) to maintain solubility of the linker payload and mixing was performed utilizing stir flasks. Conjugation was ended by the addition of an excess of 10 mM N-Acetyl Cysteine (NAC) to quench unreacted maleimide and the mixture was stirred for an additional 30 min at 25° C. Next, the ADC was incubated with activated carbon on a roller mixer at 15 rpm for 60 min at 25° C. Then, the carbon was pelleted by centrifugation for 10 min at 4,000×g. Conjugates (yields, 93-97%) were exchanged into the final formulation buffer (PBS containing 0.1 M arginine, pH 7.4), filtered through 0.22 μm polyethersulfone (PES) membranes, and stored at ≤−60° C.

RP-HPLC of the ADCs was used to determine the drug to antibody ratio (DAR) of each ADC (summarized in Table 2). The HPLC conditions used to determine the DAR are summarized below:
RP-HPLC conditions: Column—Phenomenex Kinetex 100 Å, 50×4.6 mm, 2.6 μm, Part Number: PL1912-1502. MPA—0.1% TFA/H20, MPB—0.1% TFA/CAN
Method: Flow rate—1 mL/min, Gradient—see Table 4. Column temp.—50° C.
Sample temperature—room temperature. DAD 214 nm, BW 16 nm; Reference 440 nm, BW 80 nm; Peak width >0.4 min (8 s response time (0.62 Hz); Spectrum: 200-600 nm, step 1.2 nm, slit 8 nm.
Sample—neat injection, ˜5 μg.

TABLE 4
RP-HPLC Method Flow Rate Gradient

| Time (min) | B % |
|---|---|
| 0 | 30 |
| 2 | 30 |
| 10 | 41 |
| 11.5 | 90 |
| 15.5 | 90 |
| 16.5 | 30 |
| 20 | 30 |

### Example 2: Antibody and Immunoconjugate Binding to ROR2-Positive Cells

The ability of the various ADC constructs to bind the non-small cell lung carcinoma H1155 tumor cell line was measured using an in vitro cell binding assay. Log-phase growing cells were detached using Acutase® (Millipore Sigma) at 37° C., diluted into culture media and collected by centrifugation at 500×g for 3 min. The cells were resuspended in cold 2% FBS in RPMI (binding buffer) at 2×10⁶ cells/mL. Next, cells were seeded at 50 μL per well (100,000 cells) in a 96-well v-bottom plate. Antibodies were serially diluted 4-fold in binding, beginning at 240 μg/mL, and 50 μL per well was added to the cells and incubated on ice for 30 min. The samples were washed three times with 200 μL of 2% FBS in PBS (FACS buffer), resuspended in 100 μL of secondary antibody (goat anti-human Fc PE, Invitrogen cat. #12-4998-82), diluted 500-fold in binding buffer, and incubated on ice (in the dark) for 30 min. The secondary antibody solution also contained Fixable Viability Dye eFluor™ 780 (Invitrogen) diluted 200-fold. The cells were washed three times with 200 μL of FACS buffer and were fixed with 100 μL of 2% paraformaldehyde aqueous solution (PFA) (Electron Microscopy Sciences) at 25° C. (in the dark) for 10 min. The cells were washed two times with 200 μL of FACS buffer, resuspended in 200 μL of 2 mM EDTA in PBS, and stored at 4° C. in the dark until analyzed by flow cytometry. Antibody binding was quantitated by measuring the PE median fluorescence intensity (MFI) from 10,000 events of viable singlet cells. Intact, single cells were isolated and the MFI from the control sample (secondary antibody only) was subtracted.

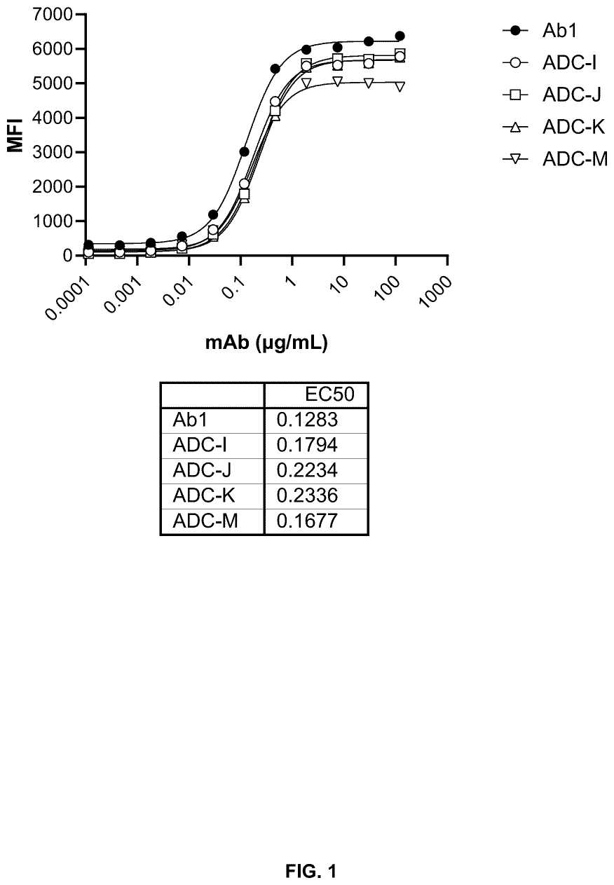

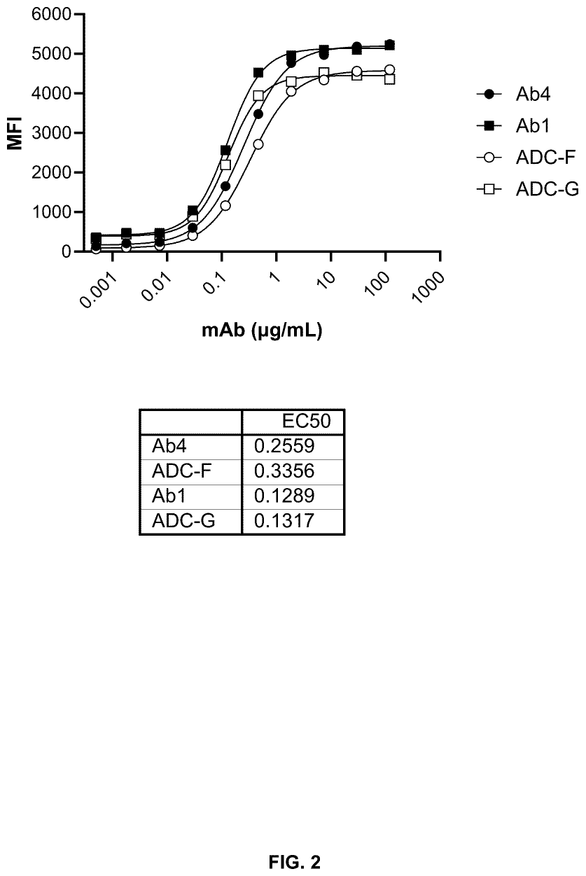

The ADCs bound H1155 cells with EC₅₀s similar to the unconjugated humanized antibody (Ab1) (FIG. 1) and to the unconjugated chimeric antibody Ab4 (FIG. 2). The ADCs tested comprised two different payloads (exatecan and duocarmycin derivative), five different linkers, and had DARs ranging from 3.2 (SLV-G) to 7.9-8 (SLV-I (Formula (VIa)), -J (Formula (VIIa)), -K (Formula (VIII.7a)), -M (Formula (IXa))).

As shown in FIG. 1, the binding EC₅₀s of linker core 1 sulfate, PAS10, and (Gly-Sar)s exatecan ADCs (SLV-I, -J, -K) are similar to antibody alone (Ab1) and to a conventional (GGFG) exatecan conjugate (SLV-M). The binding of duocarmycin derivative ADCs SLV-F and SLV-G was similar to the binding of their unlabeled parental antibodies Ab4 and Ab1, respectively, as shown in FIG. 2. All ADCs preserved binding of native antibody.

### Example 3: Antibody and Immunoconjugate Internalization on Tumor Cells

The internalization of the parental antibody and various ADC constructs to the non-small cell lung carcinoma H1155 tumor cell line was evaluated. Log-phase growing cells were detached using Acutase® (Millipore Sigma) at 37° C., diluted into culture media and collected by centrifugation at 500×g for 3 min. The cells were resuspended in cold 2% FBS in RPMI (binding buffer) at 2×10⁶ cells/mL. Next, cells were incubated with 30 μg/mL antibody on ice for 30 min. Unbound antibody was removed with three washes using ice-cold 2% FBS in PBS (FACS buffer). Following the last wash, the cells were resuspended in 1 mL ice-cold binding buffer and 100 μL (200,000 cells) was aliquoted for each time point of the internalization time course. The samples were incubated in a water bath at 37° C. for the indicated time. To terminate internalization, the cells were transferred to ice. Subsequently, the cells were collected by centrifugation at 500×g for 3 min at 4° C., the supernatant was aspirated, and the cells were resuspended in 100 μL of secondary antibody (goat anti-human Fc PE, Invitrogen cat. #12-4998-82) diluted 500-fold in binding buffer and were incubated on ice for 30 min. The secondary antibody solution also contained Fixable Viability Dye eFluor™ 780 (Invitrogen) diluted 200-fold. Samples were washed three times with 200 μL of ice-cold FACS buffer and were fixed with 100 μL of 2% paraformaldehyde aqueous solution (PFA) (Electron Microscopy Sciences) at 25° C. (in the dark) for 10 min. The cells were washed two times with 200 μL of FACS buffer, resuspended in 200 μL of 2 mM EDTA in PBS, and were stored at 4° C. in the dark until analyzed by flow cytometry. Antibody binding was quantitated by measuring the PE median fluorescence intensity (MFI) from 10,000 events of viable singlet cells. The binding signal obtained from cells incubated on ice the entire time (time 0) was designated 100% surface binding and internalization was quantitated by measuring the loss of binding signal (MFI) over time following incubation at 37° C.

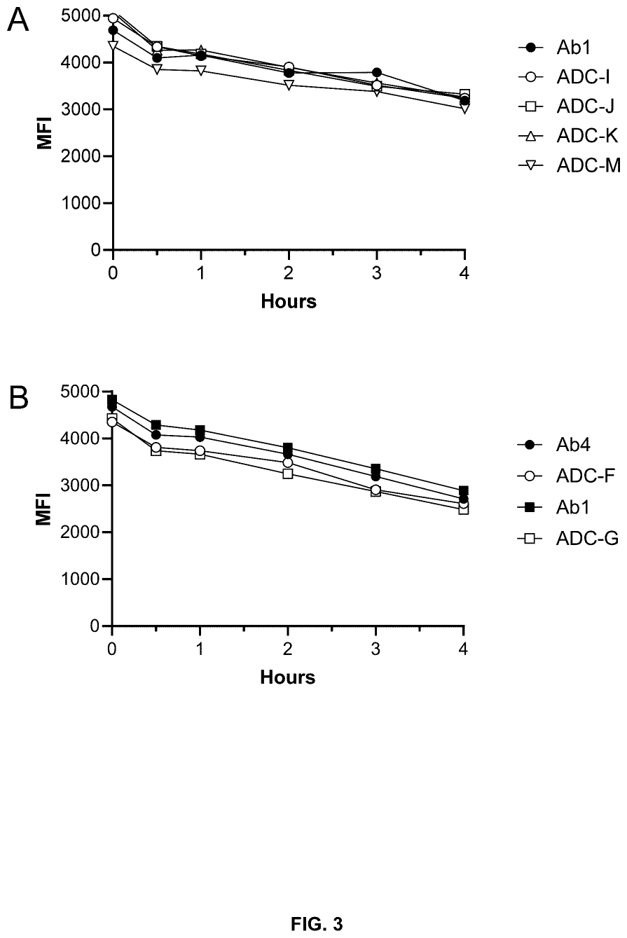

The ADCs internalized with similar rates and to the same extent as the unconjugated humanized antibody (Ab1) (FIG. 3A) and the unconjugated chimeric antibody (Ab4) (FIG. 3B). The ADCs tested comprised two different payloads (exatecan and duocarmycin derivative), five different linkers, and had DARs ranging from 3.2 (SLV-G) to 7.9-8 (SLV-I (Formula (VIa)), -J (Formula (VIIa)), -K (Formula (VIII.7a)), -M (Formula (IXa))).

### Example 4: Anti-ROR2 ADC Potency In Vitro

The potency of various ADCs was tested in vitro using multiple tumor cell lines. Different types of cancer cell lines were tested: non-small cell lung cancer (LCLC103H, H1155, H522), small cell lung cancer (H226), head and neck cancer (A253), esophageal cancer (KYSE-270), osteosarcoma (HOS, Saos-2) Ewing sarcoma (TC71), breast cancer (MDA-MB-468, MCF-7, T-47D) and colon cancer (HCT-116).

Log-phase growing cells were detached using Acutase® (Millipore Sigma) at 37° C., diluted into culture media and collected by centrifugation at 500×g for 3 min. The cells were resuspended in prewarmed (37° C.) culture media at 3.89×10⁴ cells/mL and 0.09 mL/well was transferred to a 96-well plate containing 0.01 mL of ADC. Duplicate samples of cells were incubated with 3-fold serial dilution of a particular immunoconjugate starting as high as 10 μM (10,000 nM) for 72 hours at 37° C. and 5% CO₂. After treatment, cells were incubated with an equal volume of CellTiter-Glo® reagent (Promega Inc) for 15 min at room temperature and viability was determined by reading the luminescence at all wavelengths on a SpectraMax® iD3. Percent inhibition of proliferation was calculated using treated and control samples and curves (concentration vs proliferation inhibition) and EC₅₀ values were generated in Graphpad Prism using a dose response non-linear regression fit (variable slope, constrain top=100).

Initially, the sensitivity of various non-small cell lung cancer cell lines (H520, LCLC-103H, H1155, H1975), a breast cancer cell line (SKBr3) and a chronic myelogenous leukemia cell line (K-562) to deruxtecan and exatecan was assessed. The exatecan payload was generally more potent than deruxtecan towards these cell lines, as summarized in Table 5. The potency is expressed as the mean IC₅₀ (nM) of multiple determinations (n).

TABLE 5
Payload Toxicity

| | Deruxtecan | | | Exatecan | | |
|---|---|---|---|---|---|---|
| Cell Line | Mean | SD | n | Mean | SD | n |
| H520 | 59.66 | 49.15 | 5 | 25.83 | 13.56 | 3 |
| LCLC-103H | 148.2 | 52.045 | 3 | 19.801 | 16.951 | 3 |
| H1155 | 1.56 | 0.64 | 2 | | | |
| H1975 | 6.24 | 2.78 | 4 | 0.84 | 0.21 | 4 |
| SKBr3 | 4.19 | 0.78 | 6 | 1.07 | 0.41 | 4 |
| K-562 | 105.433 | 38.279 | 3 | 13.60 | 4.350 | 3 |

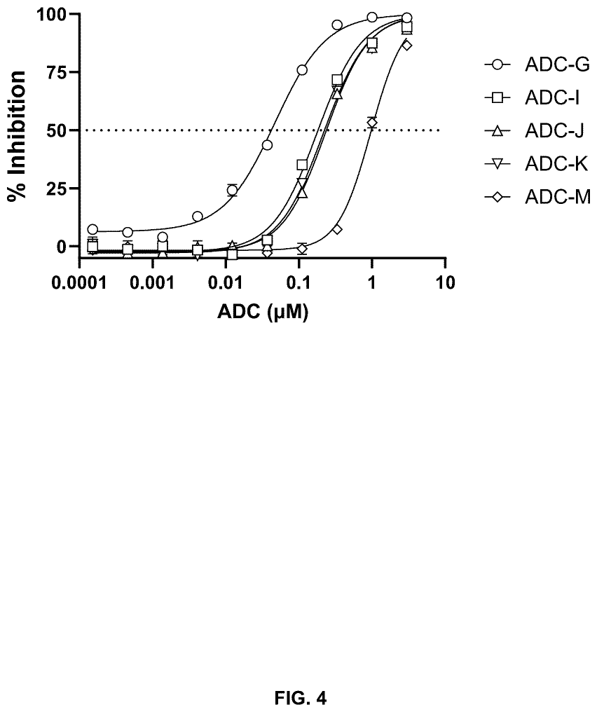

Next, ADCs employing these payloads and different linkers were characterized for cell killing activity in vitro. Representative data for ADCs SLV-G, SLV-I, SLV-J, SLV-K, and SLV-M on the non-small lung cancer derived cell line H1155 is shown in FIG. 4. For this cell line, SLV-G (duocarmycin derivative payload) was more potent than ADCs utilizing exatecan as a payload (SLV-I, SLV-J, SLV-K, and SLV-M). Of note, the duocarmycin derivative ADCs contain ˜50% as much payload per antibody as the exatecan ADCs (DAR 3.5 vs DAR 7.9-8, respectively). Additionally, exatecan-containing ADCs with novel linkers (SLV-I, SLV-J, SLV-K) were more potent than SLV-M, an ADC which employs a more conventional GGFG linker with exatecan. For experiments described herein and below, SLV-I is of Formula (VIa), SLV-J is of Formula (VIIa), and SLV-K is of Formula (VIII.7a).

A summary of the cell killing activity (IC₅₀, μM) of multiple ADC constructs towards cell lines derived from non-small cell lung cancer and squamous cell carcinoma is shown in Table 6. Anti-ROR2 ADCs displayed potent activity towards certain cell lines (LCLC-103H, H1155, and H522). The potency of SLV-B (deruxtecan payload) towards the tumor cell line H1155 was similar to ADCs with an exatecan payload and the same (SLV-M) or different (SLV-I, SLV-J, SLV-K) linkers.

TABLE 6
ADC Cytotoxicity on Lung Cancer Cell Lines. IC₅₀ (μM)

| | LCLC103H | H1155 | H522 | A549 | H226 |
|---|---|---|---|---|---|
| Construct | (NSCLC) | (NSCLC) | (NSCLC) | (NSCLC) | (SCC) |
| SLV-A | 0.13 | | | 316 | 2.547 |
| | | | | | 1.657 |
| SLV-B | | 0.113 | NE | 4.86 | 0.556 |
| | | 0.122 | >3 | | 0.276 |
| | | | 2.517 | | |
| SLV-C | 0.27 | 0.114 | 0.300 | 0.195 | 0.495 |
| | | 0.076 | 0.224 | | 0.152 |
| | | | 0.211 | | |
| SLV-F | | 0.04 | 0.42 | | |
| | | 0.04 | 0.42 | | |
| | | | 0.267 | | |
| SLV-G | | 0.016 | 0.195 | | |
| | | 0.016 | 0.198 | | |
| | | 0.041 | | | |
| | | 0.042 | | | |
| SLV-I | | 0.258 | | | |
| | | 0.183 | | | |
| SLV-J | | 0.242 | | | |
| | | 0.238 | | | |
| SLV-K | | 0.299 | | | |
| | | 0.224 | | | |
| SLV-M | | 0.819 | | | |
| | | 0.980 | | | |

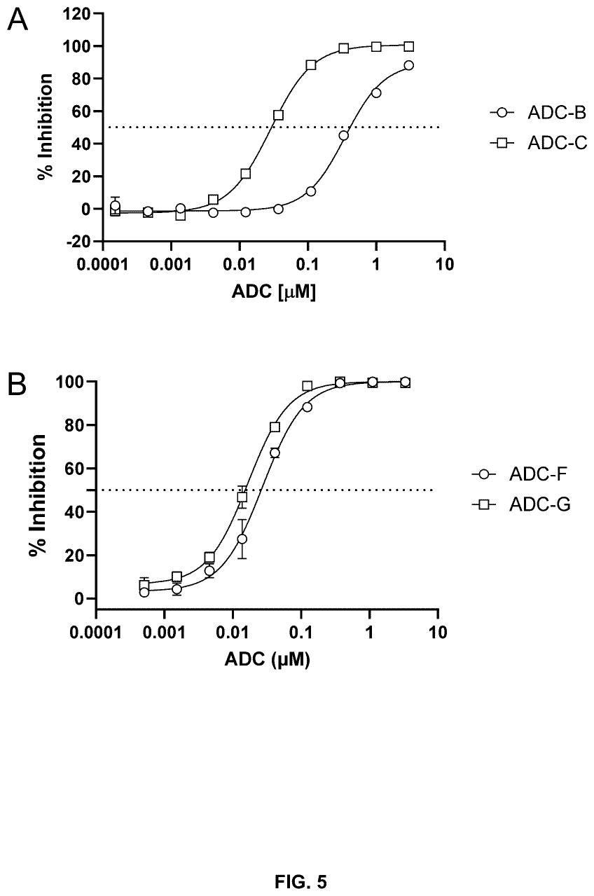

The cell killing activity of the ADCs was tested on additional cell lines derived from different tumor types. For example, representative data for ADCs SLV-B, SLV-C, SLV-F, and SLV-G on the osteosarcoma derived cell line HOS is shown in FIG. 5. ADCs with the duocarmnycin derivative payload (FIG. 5A, SLV-C; FIG. 5B, SLV-F, SLV-G) were more potent than SLV-B (FIG. 5A), which uses the deruxtecan payload. The potency of the duocarmnycin payload was similar for chimeric ROR2 antibody (SLV-C), effector-less chimeric antibody (SLV-F), and effector-less humanized ROR2 antibody (SLV-G).

A summary of the cell killing activity (IC₅₀, μM) of multiple ADC constructs towards cell lines derived from different tumor types is shown in Table 7. Anti-ROR2 ADCs displayed potent activity towards cell lines derived from different tumor types, including head & neck cancer (A253), esophageal cancer (KYSE-270), osteosarcoma (HOS, Saos-2), Ewing sarcoma (TC71), and breast cancer (MDA-MB-468). Good potency was observed with anti-ROR2 ADCs containing both deruxtecan (SLV-B) and duocarmycin derivative (SLV-C) payloads. ADCs with the duocarmycin derivative payload (SLV-C, SLV-F, SLV-G) displayed greater potency than ADCs with the deruxtecan payload (SLV-B) in in vitro assays with HOS osteosarcoma cell line. However, with other tumor cell lines (Saos-2, TC71, MDA-MB-468, HCT-116) the potency of SLV-B (deruxtecan payload) towards the tumor cell lines was similar to SLV-C(duocarmycin derivative payload).

TABLE 7
ADC Cytotoxicity on Tumor Cell Lines. IC₅₀ (μM)

| Cell Line | Tumor Type | SLV-A | SLV-B | SLV-C | SLV-F | SLV-G |
|---|---|---|---|---|---|---|
| A253 | Head & neck | 1.08 | | 0.07 | | |
| KYSE-270 | Esophageal | 0.84 | | 0.18 | | |
| HOS | Osteosarcoma | | 0.249 | 0.045 | 0.031 | 0.0135 |
| | | | 0.350 | 0.029 | 0.026 | 0.0151 |
| Saos-2 | Osteosarcoma | | 0.124 | 0.126 | | |
| | | | 0.102 | 0.049 | | |
| TC71 | Ewing | | 0.038 | 0.019 | | |
| | sarcoma | | 0.041 | 0.017 | | |
| MDA-MB-468 | Breast | 0.061 | 0.104 | 0.205 | | |
| MCF-7 | Breast | | >3 | >3 | >3 | |
| T-47D | Breast | | >3 | >3 | >3 | |
| HCT-116 | Colon | 0.111 | 0.2810.251 | 0.1640.170 | | |

### Example 5: Anti-Tumor Activities of Exemplary Immunoconjugates In Vivo

Various ADCs were tested in vivo using multiple patient-derived tumor xenograft (PDX) and tumor cell xenograft (CDX) models. For the PDX models, the tumors all expressed high levels of ROR2 mRNA and included triple negative breast cancer (TNBC, CTG-2215), sarcoma (CTG-0714, SA4094), head and neck cancer (CTG-0790), and non-small cell lung cancer (LU1656). Tumor cells were implanted subcutaneously in athymic nude (Foxn1^{nu}) mice and the mice were randomized into treatment groups when tumors reached an average size of 200 mm³. ADCs were administered intravenously (IV). Mean tumor growth inhibition (TGI) was calculated using the following formula:

$$T \text{} G \text{} \text{} I = [ \text{} 1 - \text{} ( {X}_{treated ( final )} - {X}_{treated ( day 1 )} ) \text{} / \text{} ( {X}_{control ( final )} - {X}_{control ( day 1 )} ) ] \times \text{} 100 \%$$

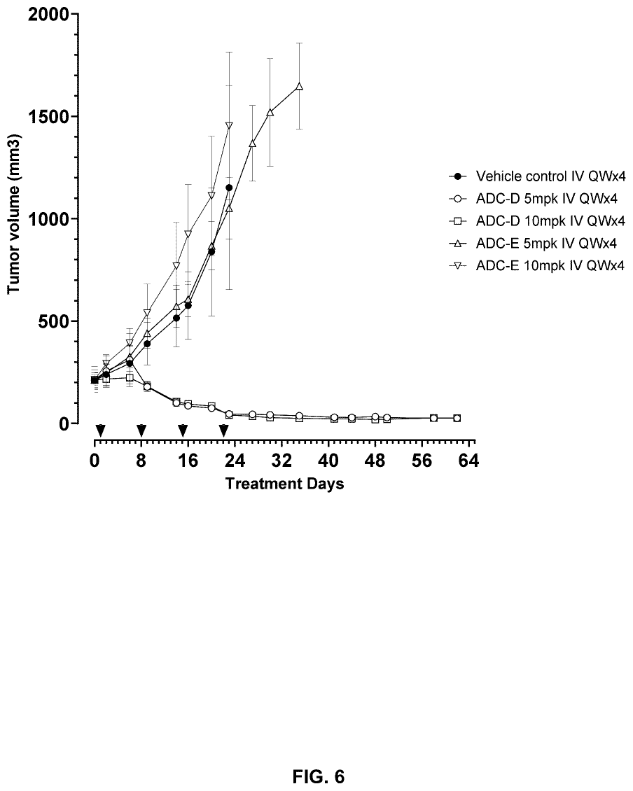

SLV-D and SLV-E were tested in a TNBC (CTG-2215) model. Treatment with SLV-D (deruxtecan payload) caused tumor regression by day 8 and complete regression was observed by day 23 (FIG. 6, open circles and open squares). In contrast, treatment with SLV-E (SN-38 payload) did not inhibit tumor growth (FIG. 6, compare closed circles with open triangles). These data demonstrate that SLV-D is active in a TNBC model and is significantly more potent than SLV-E.

SLV-D and SLV-E were tested in a head & neck cancer (CTG-0790) model. Treatment with 10 mg/kg SLV-D (deruxtecan payload) inhibited tumor growth after a single dose, while treatment with 5 mg/kg SLV-D inhibited tumor growth after the third dose. Treatment with SLV-D inhibited tumor growth by 69% and 99% at 5 mg/kg and 10 mg/kg, respectively at day 40. In contrast, treatment with SLV-E (SN-38 payload) did not inhibit tumor growth at 5 mg/kg and inhibited tumor growth by 18% at 10 mg/kg at day 40 (data summarized in Table 8). These data demonstrate that SLV-D is active in a head and neck cancer model and is significantly more potent than SLV-E.

SLV-D was tested in a highly refractory sarcoma (CTG-0714) model. Treatment with both 5 mg/kg and 10 mg/kg SLV-D (deruxtecan payload) inhibited tumor growth after a single dose. Treatment with SLV-D inhibited tumor growth by 75% and 90% at 5 mg/kg and 10 mg/kg, respectively at day 21 (data summarized in Table 8). These data demonstrate that SLV-D is active in a sarcoma model.

SLV-F (duocarmycin derivative payload) was tested in a sarcoma (SA4094) model. Treatment with 10 mg/kg and 15 mg/kg SLV-F weekly for four doses inhibited tumor growth at day 28 by 41% and 53%, respectively. Treatment with a single dose of SLV-F at either 20 mg/kg or 30 mg/kg also inhibited tumor growth, though the 20 mg/kg dose was much less effective than the 30 mg/kg dose (9% vs 64% tumor growth inhibition), as summarized in Table 8. These data demonstrate that SLV-F is also active in a sarcoma model.

SLV-F (duocarmycin derivative payload) was tested in a non-small cell lung cancer (LU1656) model. Treatment with 10 mg/kg and 15 mg/kg SLV-F weekly for four doses inhibited tumor growth at day 28 by 76% and 96%, respectively. Treatment with a single dose of SLV-F at either 20 mg/kg (88% tumor growth inhibition) or 30 mg/kg (101% tumor growth inhibition) also inhibited tumor growth, as summarized in Table 8. These data demonstrate that SLV-F is also active in a non-small cell lung cancer model.

TABLE 8
Summary of Results of Exemplary ADCs in Patient-derived Tumor Xenograft Models

| ADC(s) | Animal Model | Comments |
|---|---|---|
| SLV-D, | CTG-2215 TNBC PDX | See FIG. 6 |
| SLV-E | Dosed 5 and 10 mg/kg IV QW × 4 | Complete tumor regression observed at both |
| | | doses of SLV-D |
| | | SLV-D treated animals carried to day 62 |
| | | No tumor growth inhibition observed at either |
| | | dose of SLV-E |
| | | SN-38 payload not as effective as deruxtecan |
| SLV-D, | CTG-0790 Head & Neck PDX | 69% and 99% tumor growth inhibition at 5 and |
| SLV-E | Dosed 5 and 10 mg/kg IV QW × 4 | 10 mg/kg of SLV-D, respectively |
| | | No tumor growth inhibition and 18% tumor |
| | | growth inhibition at 5 and 10 mg/kg of SLV-E, |
| | | respectively |
| | | SN-38 payload not as effective as deruxtecan |
| SLV-D | CTG-0714 Sarcoma PDX | Highly refractory model |
| | Dosed 5 and 10 mg/kg IV QW × 4 | 90% tumor growth inhibition on day 21 at 10 |
| | | mg/kg |
| SLV-F | SA4094 Sarcoma PDX | 41% and 53% tumor growth inhibition |
| | Dosed 10 and 15 mg/kg IV QW × 4 | observed on day 28 at 10 and 15 mg/kg doses, |
| | Dosed 20 and 30 mg/kg IV once | respectively |
| | Final tumor measurement on day 28 | 9% and 64% tumor growth inhibition observed |
| | | at 20 and 30 mg/kg doses, respectively |
| SLV-F | LU1656 NSCLC PDX | 76% and 96% tumor growth inhibition |
| | Dosed 10 and 15 mg/kg IV QW × 4 | observed on day 28 at 10 and 15 mg/kg doses, |
| | Dosed 20 and 30 mg/kg IV once | respectively |
| | | 88% tumor growth inhibition observed at 20 |
| | | mg/kg dose |
| | | Slight tumor regression (101% tumor growth |
| | | inhibition) observed at 30 mg/kg dose |

For the CDX models, cell lines derived from NSCLC (LCLC-103H, H520) and chronic myeloid leukemia (CML, K562) were used. Tumor cells were implanted subcutaneously in athymic nude mice and the mice were randomized into treatment groups (5 mice/group) when tumors reached an average size of 200 mm³.

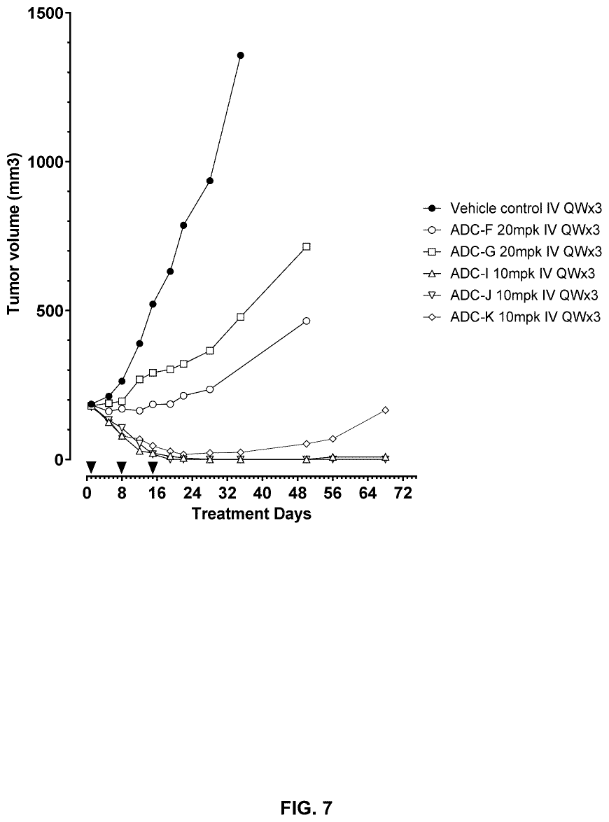

SLV-F, SLV-G, SLV-I, SLV-J and SLV-K were tested in a NSCLC (LCLC-103H tumor cell line) xenograft model. SLV-F and SLV-G utilize a duocarmycin derivative payload and have a DAR ˜3.2-3.5 while SLV-I, SLV-J and SLV-K utilize an exatecan payload and have a DAR ˜7.9-8. To treat with similar amounts of payload, animals receiving SLV-F and SLV-G were dosed at twice the concentration (20 mg/kg) of ADC as the mice treated with SLV-I, SLV-J, and SLV-K (10 mg/kg). Treatment with SLV-I (core 1 sulfate linker) and SLV-J (core 1 PAS10 linker) resulted in complete tumor regression through day 68 (FIG. 7, open triangles). Treatment with SLV-K (core 1 (Gly-Sar)₅ linker) also caused complete tumor regression through day 35 (FIG. 7, open diamonds). In contrast, treatment with SLV-F and SLV-G inhibited tumor growth partially through day 48 (FIG. 7, open circles and open squares). These data demonstrate that all of these ADCs are active in a NSCLC model. In addition, the ADCs containing the exatecan payload were more potent in this model than the ADCs containing the duocarmycin derivative payload.

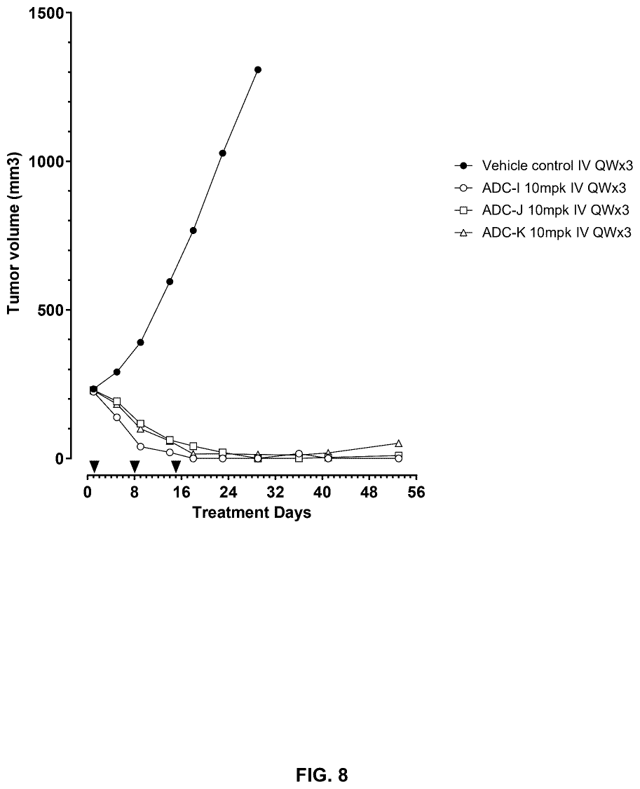

SLV-I, SLV-J, and SLV-K were tested in the same NSCLC (LCLC-103H tumor cell line) xenograft model. Treatment with all of these ADCs resulted in complete tumor regression through day 41 (FIG. 8). Animals treated with SLV-K displayed modest regrowth of tumor observed at day 53 (FIG. 8, open triangles). These data demonstrate that all of the ADCs with the core 1 linker system (SLV-I, SLV-312, SLV-K) are active in a NSCLC model. Furthermore, the core 1 linker derivatives containing sulfate (SLV-I), PAS10 (SLV-J) and (Gly-Sar)₅ (SLV-K) display comparable potencies in this model.

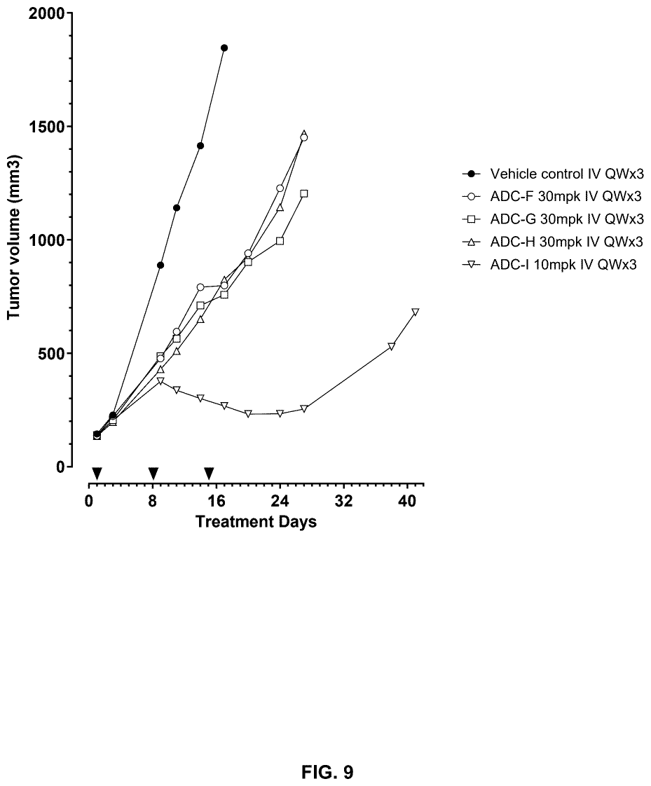

SLV-F, SLV-G, SLV-H, and SLV-I were tested in a different NSCLC (H520 tumor cell line) xenograft model. SLV-F, SLV-G, and SLV-H utilize a duocarmycin derivative payload and have a DAR ˜3.2-3.5 while SLV-I utilizes an exatecan payload and has a DAR=8. To normalize the amount of payload, the animals being treated with SLV-F, SLV-G and SLV-H were dosed at 30 mg/kg of ADC, while SLV-I was dosed at 10 mg/kg. Treatment with all of these ADCs resulted in tumor growth inhibition (FIG. 9). However, the greatest tumor growth inhibition was observed with SLV-I (exatecan payload) as opposed to ADCs utilizing duocarmycin derivative as payload (SLV-F, SLV-G, SLV-H; FIG. 9, compare open inverted triangles versus open circles, open squares, and open triangles). Animals treated with ADCs containing the duocarmycin derivative payload conjugated to different forms of the ROR2 antibody displayed similar tumor growth inhibition. Specifically, ADCs with the chimeric ROR2 antibody (SLV-F), humanized ROR2 antibody version 1 (SLV-G), and humanized ROR2 antibody version 2 (SLV-H) all inhibited tumor growth to a similar extent (FIG. 9, compare open circles, open squares, open triangles).

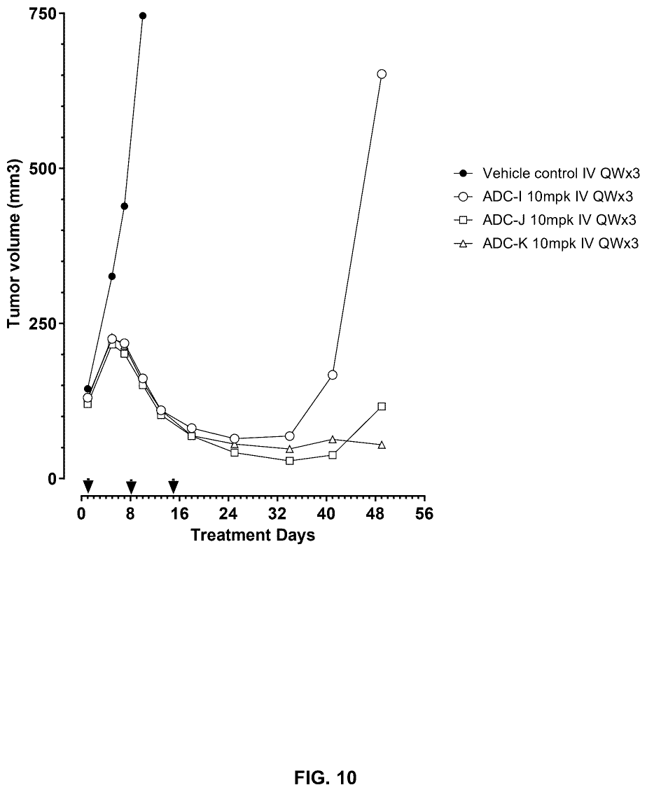

SLV-I, SLV-J, and SLV-K were tested in another NSCLC (H520 tumor cell line) xenograft model. Treatment with all of these ADCs resulted in tumor regression through day 34 (FIG. 10). Animals treated with SLV-J began to display regrowth of tumor after day 41 (FIG. 10, open squares) while animals treated with SLV-I (sulfate modified linker) displayed rapid regrowth of tumor after day 34 (FIG. 10, open circles). These data demonstrate that all of the ADCs with the core 1 linker system (SLV-I, SLV-J, SLV-K) are active in a second NSCLC model.

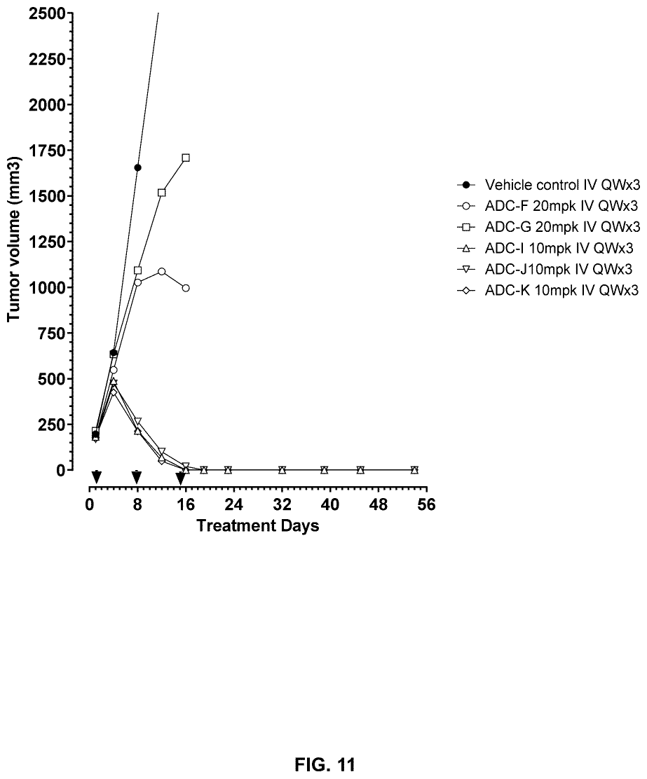

SLV-F, SLV-G, SLV-I, SLV-J and SLV-K were tested in a chronic myeloid leukemia (CML) xenograft model (K562 tumor cell line). SLV-F and SLV-G utilize a duocarmycin derivative payload and have a DAR ˜3.2-3.5 while SLV-I, SLV-J and SLV-K utilize an exatecan payload and have a DAR ˜7.9-8. To treat with similar amounts of payload, animals receiving SLV-F and SLV-G were dosed at twice the concentration (20 mg/kg) of ADC as the mice treated with SLV-I, SLV-J, and SLV-K (10 mg/kg). Treatment with SLV-I, SLV-J and SLV-K resulted in complete tumor regression through day 54 (FIG. 11, open triangles, open inverted triangles, open diamonds). In contrast, treatment with SLV-F and SLV-G inhibited tumor growth partially through day 16 (FIG. 11, open circles and open squares). These data demonstrate that all of the ADCs are active in a CML model. However, the ADCs containing exatecan payload were significantly more efficacious in this model than the ADCs containing the duocarmycin derivative payload.

A summary of the results of tumor xenograft models is shown in Table 9.

TABLE 9
Summary of Results of Exemplary ADCs in Tumor Cell Line Xenograft Models

| ADC(s) | Animal Model | Comments |
|---|---|---|
| SLV-F, | NSCLC (LCLC-103H) CDX | See FIG. 7 |
| SLV-G, | Dosed SLV-F, SLV-G at 20 mg/kg | Complete tumor regression observed with |
| SLV-I, | IV QW × 3; SLV-I, SLV-J, SLV-K | ADCs containing exatecan payload (SLV-I, |
| SLV-J, | at 10 mg/kg IV QW × 3 | SLV-J, SLV-K) |
| SLV-K | | Tumor growth inhibition observed with ADCs |
| | | containing duocarmycin derivative payload |
| | | (SLV-F, SLV-G) |
| | | Exatecan payload/linker system more effective |
| | | than duocarmycin derivative payload/linker |
| | | system |
| | | Less potent payload works better than more |
| | | potent payload |
| SLV-I, | NSCLC (LCLC-103H) CDX | See FIG. 8 |
| SLV-J, | Dosed at 10 mg/kg IV QW × 3 | Complete tumor regression observed with all |
| SLV-K | | ADCs |
| SLV-F, | NSCLC (H520) CDX | See FIG. 9 |
| SLV-G, | Dosed SLV-F, SLV-G, SLV-H at | Greatest tumor growth inhibition observed |
| SLV-H, | 30 mg/kg IV QW × 3; SLV-I at 10 | with SLV-I (exatecan payload) |
| SLV-I | mg/kg IV QW × 3 | Equivalent tumor growth inhibition observed |
| | | with SLV-F, SLV-G, SLV-H (duocarmycin |
| | | derivative payload) |
| | | No difference between chimeric (SLV-F) and |
| | | two different humanized versions of mAb 6E6 |
| | | (SLV-G, SLV-H) |
| | | Less potent payload works better than more |
| | | potent payload |
| SLV-I, | NSCLC (H520) CDX | See FIG. 10 |
| SLV-J, | Dosed at 10 mg/kg IV QW × 3 | Tumor regression observed with all ADCs |
| SLV-K | | Faster tumor regrowth observed with sulfate |
| | | modified linker (SLV-I) |
| SLV-F, | CML (K562) CDX | See FIG. 11 |
| SLV-G, | Dosed SLV-F, SLV-G at 20 mg/kg | Complete tumor regression observed with |
| SLV-I, | IV QW × 3; SLV-I, SLV-J, SLV-K | ADCs containing exatecan payload (SLV-I, |
| SLV-J, | at 10 mg/kg IV QW × 3 | SLV-J, SLV-K) |
| SLV-K | | Modest tumor growth inhibition observed with |
| | | ADCs containing duocarmycin derivative |
| | | payload (SLV-F, SLV-G) |
| | | Exatecan payload/linker system more effective |
| | | than duocarmycin derivative payload/linker |
| | | system |

### SEQUENCES

Sequences described in the present disclosure are provided below.

| SEQ | | |
|---|---|---|
| ID | | |
| NO: | Name of Sequence | Sequence |
| 1 | Ab1 HC | EVQLVESGGGLVKPGGSLRLSCAASGFTFSTYGVSWV |
| | | RQAPGKGLEWVSTISSGGGYTHYAGSVKGRFTISRDN |
| | | AKNSLYLQMNSLRAEDTAVYYCARHPRDFSYALDYWG |
| | | QGTTVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKKVEPKS |
| | | CDKTHTCPPCPAPEAAGGPSVFLFPPKPKDTLMISRT |
| | | PEVTCVVVDVSHEDPEVKFNWYVDGVEVHNAKTKPRE |
| | | EQYNSTYRVVSVLTVLHQDWLNGKEYKCKVSNKALAA |
| | | PIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKSL |
| | | SLSPGK |
| 2 | Ab1/Ab2 LC | EIVMTQSPATLSVSPGERATLSCRASQDVGHYLAWYQ |
| | | QKPGQAPRLLIYWASTRATGIPARFSGSGSGTEFTLT |
| | | ISSLQSEDFAVYYCQQYNIYPWTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| 3 | Ab1 V_{H} | EVQLVESGGGLVKPGGSLRLSCAASGFTFSTYGVSWV |
| | | RQAPGKGLEWVSTISSGGGYTHYAGSVKGRFTISRDN |
| | | AKNSLYLQMNSLRAEDTAVYYCARHPRDFSYALDYWG |
| | | QGTTVTVSS |
| 4 | Ab1/Ab2 V_{L} | EIVMTQSPATLSVSPGERATLSCRASQDVGHYLAWYQ |
| | | QKPGQAPRLLIYWASTRATGIPARFSGSGSGTEFTLT |
| | | ISSLQSEDFAVYYCQQYNIYPWTFGQGTKVEIK |
| 5 | Ab1 HCDR1 (IMGT) | GFTFSTYG |
| 6 | Ab1/Ab2/Ab3/Ab4 HCDR2 | ISSGGGYT |
| | (IMGT) | |
| 7 | Ab1 HCDR3 (IMGT/Aho) | ARHPRDFSYALDY |
| 8 | Ab1/Ab2/Ab3/Ab4 LCDR1 | QDVGHY |
| | (IMGT) | |
| 9 | Ab1/Ab2/Ab3/Ab4 LCDR2 | WAS |
| | (IMGT) | |
| 10 | Ab1/Ab2/Ab3/Ab4 LCDR3 | QQYNIYPWT |
| | (IMGT/Kabat/Chothia/Aho) | |
| 11 | Ab2 HC | EVQLVESGGGLVKPGGSLRLSCAASGFTFSQYGHSWV |
| | | RQAPGKGLEWVSTISSGGGYTHYAHSVKGRFTISRDN |
| | | AKNSLYLQMNSLRAEDTAVYYCARHPRDFSYANDYWG |
| | | QGTTVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKKVEPKS |
| | | CDKTHTCPPCPAPEAAGGPSVFLFPPKPKDTLMISRT |
| | | PEVTCVVVDVSHEDPEVKFNWYVDGVEVHNAKTKPRE |
| | | EQYNSTYRVVSVLTVLHQDWLNGKEYKCKVSNKALAA |
| | | PIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKSL |
| | | SLSPGK |
| 12 | Ab2 V_{H} | EVQLVESGGGLVKPGGSLRLSCAASGFTFSQYGHSWV |
| | | RQAPGKGLEWVSTISSGGGYTHYAHSVKGRFTISRDN |
| | | AKNSLYLQMNSLRAEDTAVYYCARHPRDFSYANDYWG |
| | | QGTTVTVSS |
| 13 | Ab2 HCDR1 (IMGT) | GFTFSQYG |
| 14 | Ab2 HCDR3 (IMGT) | ARHPRDFSYANDY |
| 15 | Ab3 HC | EVQLVESGGDLVKPGGSLKLSCAASGFTFSNYGMSWV |
| | | RQTPDKRLEWVATISSGGGYTHYVDSVKGRFTISRDN |
| | | ANHILYLQMSSLNSEDTAMYYCARHPRDFSYAMDYWG |
| | | QGTSVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKKVEPKS |
| | | CDKTHTCPPCPAPELLGGPSVFLFPPKPKDTLMISRT |
| | | PEVTCVVVDVSHEDPEVKFNWYVDGVEVHNAKTKPRE |
| | | EQYNSTYRVVSVLTVLHQDWLNGKEYKCKVSNKALPA |
| | | PIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKSL |
| | | SLSPGK |
| 16 | Ab3/Ab4 LC | DIVMTQSHKFMSTSIGDRVSITCKASQDVGHYVAWYQ |
| | | QKPGQSPKLLIYWASTRHTGVPDRFTGSGSGTDFTLT |
| | | ISNVQSEDLADYFCQQYNIYPWTFGGGSKLAIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| 17 | Ab3/Ab4 V_{H} | EVQLVESGGDLVKPGGSLKLSCAASGFTFSNYGMSWV |
| | | RQTPDKRLEWVATISSGGGYTHYVDSVKGRFTISRDN |
| | | ANHILYLQMSSLNSEDTAMYYCARHPRDFSYAMDYWG |
| | | QGTSVTVSS |
| 18 | Ab3/Ab4 V_{L} | DIVMTQSHKFMSTSIGDRVSITCKASQDVGHYVAWYQ |
| | | QKPGQSPKLLIYWASTRHTGVPDRFTGSGSGTDFTLT |
| | | ISNVQSEDLADYFCQQYNIYPWTFGGGSKLAIK |
| 19 | Ab3/Ab4 HCDR1 (IMGT) | GFTFSNYG |
| 20 | Ab3/Ab4 HCDR3 | ARHPRDFSYAMDY |
| | (IMGT) | |
| 21 | Ab4 HC | EVQLVESGGDLVKPGGSLKLSCAASGFTFSNYGMSWV |
| | | RQTPDKRLEWVATISSGGGYTHYVDSVKGRFTISRDN |
| | | ANHILYLQMSSLNSEDTAMYYCARHPRDFSYAMDYWG |
| | | QGTSVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKKVEPKS |
| | | CDKTHTCPPCPAPEAAGGPSVFLFPPKPKDTLMISRT |
| | | PEVTCVVVDVSHEDPEVKFNWYVDGVEVHNAKTKPRE |
| | | EQYNSTYRVVSVLTVLHQDWLNGKEYKCKVSNKALAA |
| | | PIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKSL |
| | | SLSPGK |
| 22 | Ab1 HCDR1 (Kabat) | TYGVS |
| 23 | Ab1 HCDR2 (Kabat) | TISSGGGYTHYAGSVKG |
| 24 | Ab1 HCDR3 | HPRDFSYALDY |
| | (Kabat/Chothia) | |
| 25 | Ab1 LCDR1 | RASQDVGHYLA |
| | (Kabat/Chothia/Aho) | |
| 26 | Ab1 LCDR2 | WASTRAT |
| | (Kabat/Chothia) | |
| 27 | Ab1 HCDR1 (Chothia) | GFTFSTY |
| 28 | Ab1 HCDR2 (Chothia) | SSGGGY |
| 29 | Ab1 HCDR1 (AHo) | AASGFTFSTYGVS |
| 30 | Ab1 HCDR2 (AHo) | TISSGGGYTH |
| 31 | Ab1 LCDR2 (AHo) | YWASTRAT |
| 32 | Ab1 HCDR1 (Contact) | STYGVS |
| 33 | Ab1 HCDR2 (Contact) | WVSTISSGGGYTH |
| 34 | Ab1 HCDR3 (Contact) | ARHPRDFSYALD |
| 35 | Ab1 LCDR1 (Contact) | GHYLAWY |
| 36 | Ab1 LCDR2 (Contact) | LLIYWASTRA |
| 37 | Ab1 LCDR3 (Contact) | QQYNIYPW |
| 38 | Human ROR2 | MARGSALPRRPLLCIPAVWAAAALLLSVSRTSGEVEV |
| | | LDPNDPLGPLDGQDGPIPTLKGYFLNFLEPVNNITIV |
| | | QGQTAILHCKVAGNPPPNVRWLKNDAPVVQEPRRIII |
| | | RKTEYGSRLRIQDLDTTDTGYYQCVATNGMKTITATG |
| | | VLFVRLGPTHSPNHNFQDDYHEDGFCQPYRGIACARF |
| | | IGNRTIYVDSLQMQGEIENRITAAFTMIGTSTHLSDQ |
| | | CSQFAIPSFCHFVFPLCDARSRTPKPRELCRDECEVL |
| | | ESDLCRQEYTIARSNPLILMRLQLPKCEALPMPESPD |
| | | AANCMRIGIPAERLGRYHQCYNGSGMDYRGTASTTKS |
| | | GHQCQPWALQHPHSHHLSSTDFPELGGGHAYCRNPGG |
| | | QMEGPWCFTQNKNVRMELCDVPSCSPRDSSKMGILYI |
| | | LVPSIAIPLVIACLFFLVCMCRNKQKASASTPQRRQL |
| | | MASPSQDMEMPLINQHKQAKLKEISLSAVRFMEELGE |
| | | DRFGKVYKGHLFGPAPGEQTQAVAIKTLKDKAEGPLR |
| | | EEFRHEAMLRARLQHPNVVCLLGVVTKDQPLSMIFSY |
| | | CSHGDLHEFLVMRSPHSDVGSTDDDRTVKSALEPPDF |
| | | VHLVAQIAAGMEYLSSHHVVHKDLATRNVLVYDKLNV |
| | | KISDLGLFREVYAADYYKLLGNSLLPIRWMAPEAIMY |
| | | GKFSIDSDIWSYGVVLWEVFSYGLQPYCGYSNQDVVE |
| | | MIRNRQVLPCPDDCPAWVYALMIECWNEFPSRRPRFK |
| | | DIHSRLRAWGNLSNYNSSAQTSGASNTTQTSSLSTSP |
| | | VSNVSNARYVGPKQKAPPFPQPQFIPMKGQIRPMVPP |
| | | PQLYVPVNGYQPVPAYGAYLPNFYPVQIPMQMAPQQV |
| | | PPQMVPKPSSHHSGSGSTSTGYVTTAPSNTSMADRAA |
| | | LLSEGADDTQNAPEDGAQSTVQEAEEEEEGSVPETEL |
| | | LGDCDTLQVDEAQVQLEA |
| 39 | GGFG linker | GGFG |
| 40 | Ab1 HCDR1 | TY |
| 41 | Ab1 LCDR1 | GHY |

1. An immunoconjugate having the formula of Ab−((L)m−(D))n, wherein:
Ab is an antibody or an antigen-binding portion thereof that specifically binds to human receptor tyrosine kinase like orphan receptor 2 (ROR2);
L is a linker;
m is 0 or 1;
D is a cytotoxic drug moiety; and
n is an integer from 1 to 10.

2. The immunoconjugate of claim 1, wherein the antibody or antigen-binding portion competes or cross-competes for binding to human ROR2 or binds to the same human ROR2 epitope as an antibody that comprises a heavy chain (HC) and a light chain (LC) comprising
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively.

3. The immunoconjugate of claim 1, wherein the antibody or antigen-binding portion comprises heavy chain complementarity-determining region (CDR) 1-3 (HCDR1-3) and light chain CDR1-3 (LCDR1-3) amino acid sequences of
a) SEQ ID NOs: 5, 6, 7, 8, 9, and 10, respectively;
b) SEQ ID NOs: 13, 6, 14, 8, 9, and 10, respectively; or
c) SEQ ID NOs: 19, 6, 20, 8, 9, and 10, respectively.

4. The immunoconjugate of claim 1, wherein the antibody or antigen-binding portion comprises
HCDR1 comprising SEQ ID NO: 40;
HCDR2 comprising SEQ ID NO: 28;
HCDR3 comprising SEQ ID NO: 24;
LCDR1 comprising SEQ ID NO: 41;
LCDR2 comprising SEQ ID NO: 9; and
LCDR3 comprising SEQ ID NO: 37.

5. The immunoconjugate of claim 3, wherein the antibody or antigen-binding portion comprises heavy chain variable domain (V_{H}) and light chain variable domain (V_{L}) amino acid sequences of
a) SEQ ID NOs: 3 and 4, respectively;
b) SEQ ID NOs: 12 and 4, respectively; or
c) SEQ ID NOs: 17 and 18, respectively.

6. The immunoconjugate of claim 3, wherein the antibody is of isotype IgG,
optionally wherein the antibody is of isotype subclass IgG₁, IgG₂, IgG₃, or IgG₄,
further optionally wherein the Fc region of the antibody comprises one or more mutations that reduce effector function.

7-8. (canceled)

9. The immunoconjugate of claim 3, wherein the antibody comprises HC and LC amino acid sequences of
a) SEQ ID NOs: 1 and 2, respectively;
b) SEQ ID NOs: 11 and 2, respectively;
c) SEQ ID NOs: 15 and 16, respectively; or
d) SEQ ID NOs: 21 and 16, respectively;
optionally wherein the HC amino acid sequence lacks the C-terminal lysine.

10. The immunoconjugate of claim 3, wherein the antigen-binding portion is a Fab, F(ab)₂, or scFv.

11. The immunoconjugate of claim 3, wherein the cytotoxic drug moiety is an anti-tubulin agent or a topoisomerase I inhibitor.

12. The immunoconjugate of claim 11, wherein the cytotoxic drug moiety comprises a chemical structure selected from the group consisting of monomethyl auristatin E (MMAE), monomethyl auristatin F (MMAF), exatecan, an exatecan derivative (DXd), and SN-38; or a pharmaceutically acceptable salt, analog, prodrug, or if appropriate, ester thereof.

13. The immunoconjugate of claim 3, wherein the linker comprises a cleavable moiety.

14. The immunoconjugate of claim 3, wherein the immunoconjugate comprises
a) a linker comprising one or more of valine-alanine (VA), valine-citrulline (VC), C₅—C(═O)—VC, para-substituted phenylene-C₃ (Ph(p)-C₃), meta-substituted phenylene-C₂ (Ph(m)-C₂), C₅ alkyl, para-aminobenzyloxycarbonyl (PAB), amino methylene (AM), and GGFG (SEQ ID NO: 39); or
b) a formula selected from Formulae (I), (Ia), (Ib), (Ic), (Id), (Ie), (If), (II), (IIa), (IIb), (IIc), (IId), (IIe), and (IIf), or a pharmaceutically acceptable salt, or if appropriate, ester, thereof.

15. The immunoconjugate of claim 14, wherein the immunoconjugate comprises
a) a linker comprising 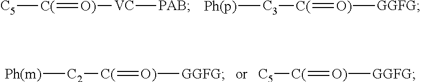
b) a formula selected from Formulae (I), (Ia), (Ib), (Ic), (Id), (Ie), and (If), where m is 2 and Y is a valine-alanine dipeptide, or a pharmaceutically acceptable salt, or if appropriate, ester, thereof, or
c) a formula selected from Formulae (II), (IIa), (IIb), (IIc), (IId), (IIe), and (IIf), or a pharmaceutically acceptable salt, or if appropriate, ester, thereof, wherein X comprises Formula (A), (B), or (C).

16. The immunoconjugate of claim 3, wherein the immunoconjugate comprises
a) Formula (IIIa), Formula (IVa), Formula (Va), Formula (IXa), Formula (Xa), or Formula (XIa), or a pharmaceutically acceptable salt, or if appropriate, ester, thereof; or
b) Formula (VIa), Formula (VIa.1), Formula (VIa.2), Formula (VIIa), Formula (VIIa.1), Formula (VIIa.2), Formula (VIII.1a), Formula (VIII.2a), Formula (VIII.3a), Formula (VIII.4a), Formula (VIII.5a), Formula (VIII.6a), or Formula (VIII.7a), or a pharmaceutically acceptable salt, or if appropriate, ester, thereof.

17. The immunoconjugate of claim 3, wherein the n is 3 to 8.

18. A pharmaceutical composition comprising the immunoconjugate of claim 3 and a pharmaceutically acceptable excipient,
optionally wherein the composition comprises an additional therapeutic agent selected from the group consisting of an immunomodulatory agent, a chemotherapeutic agent, an anti-neoplastic agent, and an anti-angiogenic agent.

19. (canceled)

20. A method of treating cancer in a human patient in need thereof, comprising administering to the patient a therapeutically effective amount of the immunoconjugate of claim 3, optionally wherein the cancer expresses ROR2.

21. (canceled)

22. The method of claim 20, wherein the cancer is selected from the group consisting of head and neck cancer, non-small cell lung cancer, esophageal cancer, gastric cancer, hepatic cancer, pancreatic cancer, colorectal cancer, breast cancer, endometrial cancer, ovarian cancer, soft-tissue sarcoma, bladder cancer, prostate cancer, renal cancer, and melanoma.

23. The method of claim 20, further comprising administering to the patient an additional therapeutic agent, optionally wherein the additional therapeutic agent is selected from the group consisting of an immunomodulatory agent, a chemotherapeutic agent, an anti-neoplastic agent, an anti-angiogenic agent, or a tumor vaccine.

24-26. (canceled)

27. A method of making an immunoconjugate, comprising:
providing an antibody or an antigen-binding portion thereof that specifically binds to human receptor tyrosine kinase like orphan receptor 2 (ROR2);
conjugating to the antibody or antigen-binding portion a cytotoxic drug moiety selected from the group consisting of monomethyl auristatin E (MMAE), monomethyl auristatin F (MMAF), exatecan, an exatecan derivative (DXd), and SN-38; or a pharmaceutically acceptable salt, analog, prodrug, or if appropriate, ester thereof,
wherein the antibody or antigen-binding portion is as defined in claim 3.
